(19) United States
(12) Patent Application Publication
(10) Pub. No.: US 2024/0197759 A1
(43) Pub. Date: Jun. 20, 2024

(54) IMMUNE STIMULATING NANOPARTICLE COMPOSITION

(71) Applicants: DANMARKS TEKNISKE UNIVERSITET, Kongens Lyngby (DK); MONTA BIOSCIENCES APS, Kongens Lyngby (DK)

(72) Inventors: Jonas Rosager HENRIKSEN, Kongens Lyngby (DK); Thomas Lars ANDRESEN, Kongens Lyngby (DK); Simon Skjøde JENSEN, Brønshøj (DK); Ladan PARHAMIFAR, Kongens Lyngby (DK); Rasmus Dithmar MÜNTER, Kongens Lyngby (DK); Anders E. HANSEN, Kongens Lyngby (DK); Camilla STAVNSBJERG, Kongens Lyngby (DK); Esben CHRISTENSEN, Kongens Lyngby (DK)

(21) Appl. No.: 18/551,741

(22) PCT Filed: Mar. 25, 2022
(86) PCT No.: PCT/EP2022/057889

§ 371 Date: Sep. 21, 2023

(30) Foreign Application Priority Data
Mar. 25, 2021 (EP) 21164973.6

(51) Int. Cl. A61K 31/675; A61K 9/107; A61K 9/127; A61K 9/51; A61K 31/4745; A61K 45/06; A61P 35/00

(52) U.S. Cl. CPC A61K 31/675; A61K 9/1075; A61K 9/1271; A61K 9/5146; A61K 31/4745; A61K 45/06; A61P 35/00

(57) ABSTRACT

The present invention relates to immune stimulating nanoparticle compositions, and their use in treatment of diseases and disorders, such as cancer. In particular, the present invention relates to nanoparticle compositions comprising a TLR7 agonist, such as 1V270.5

### TECHNICAL FIELD

The present invention relates to immune stimulating nanoparticle compositions, and their use in treatment of diseases and disorders, such as cancer. In particular, the present invention relates to micelle compositions comprising a TLR7 agonist, such as 1V270.

### BACKGROUND

Toll-like receptors (TLRs) are a class of receptors expressed on various cell types and play a key role in the innate immune system. Upon activation, TLRs activate signal transduction pathway involved in immune activation. Several mammalian TLRs and a number of their agonists have been identified. For example, guanine and uridine rich single-stranded RNA has been identified as a natural ligand for TLR7. In addition, several low molecular weight activators of TLR7 have been identified, including imidazoquinolines, and purine-like molecules. While TLR stimulation initiates a common signaling cascade (involving the adaptor protein MyD88, the transcription factor NFκB, and proinflammatory and effector cytokines), different TLRs are expressed by different cell types, however, TLR7 is mainly expressed in monocytes, plasmacytoid dendritic cells, myeloid dendritic cells and B-cells and are localized to the endosome membrane.

TLR7 has been shown to play a significant role in the pathogenesis of autoimmune disorders such as Systemic Lupus Erythematosus (SLE) as well as in the regulation of antiviral immunity. A TLR7 agonist, Aldara (Imiquimod), an imidazoquinoline, has been approved for topical use in treating warts caused by papillomavirus, for basal cell carcinoma and actinic keratoses. Due to their ability to induce robust production of anti-cancer cytokines such as interleukin-12, TLR7 agonists have also been investigated for cancer immunotherapy. Recent examples include TMX-202 delivery via liposomal formulation, as well as the delivery of resiquimod via nanoparticles formed from beta-cyclodextrin.

However, repeated injections of therapeutic nanoparticle compositions have been shown to trigger an accelerated blood clearance (ABC) phenomenon by antibodies, in particular for PEG-based liposomes, which tampers the utility of therapeutic nanoparticle delivery systems. Hence, there is an impetus in the art for the development of more effective therapeutic nanoparticle compositions.

### SUMMARY

The present inventors have surprisingly discovered that nanoparticle compositions comprising a TLR7 agonist show very potent anti-cancer activity and are devoid of previously recognized undesired pharmacokinetic behaviour.

In a first aspect, the present disclosure provides a micelle composition comprising: a toll-like receptor 7 (TLR7) agonist of formula (I), formula (II), formula (III) or formula (IV);

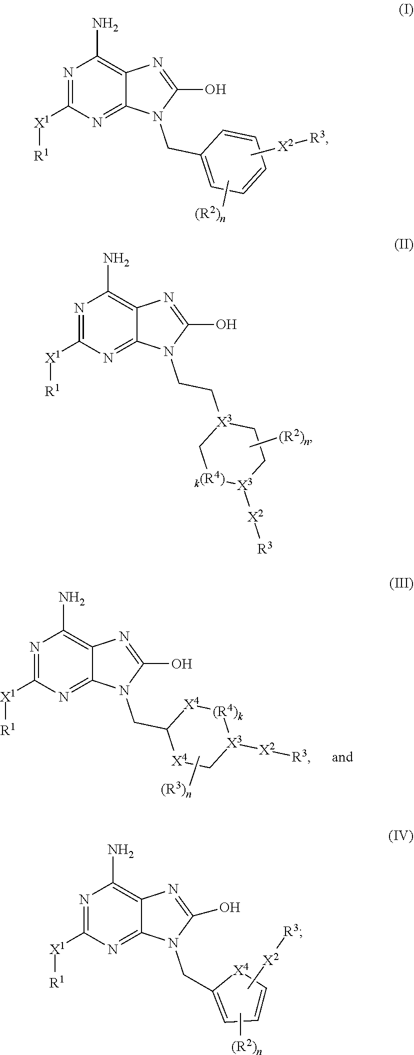

wherein X is —O—, —S—, or —NR^{C};
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R^{C} is hydrogen, C₁₋₁₀alkyl, or substituted C₁₋₁₀alkyl; or R^{C} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxy C₁₋₆alkylene, amino, cyano, halo, or aryl;
n is 0, 1, 2, 3 or 4;
X² is a bond or a linking group; and
R³ is a lipid;

### X³ is —N— or —CH—;

R⁴ is —CH₂— or —CH(R²)—; and
k is 0 or 1;
X⁴ is —O—, —S—, —NH—, —N(R^{d})—, —CH₂—, or —CH(R²)—;
each R^{d} is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)O(C₁-C₆)alkyl (alkoxycarbonyl), substituted —C(O)O(C₁-C₆)alkyl, —C(O)NR^{a}R^{b} (carbamoyl); or a tautomer thereof;
or a pharmaceutically acceptable salt or solvate thereof, and
wherein the ring system of formula (II) is a piperidine ring with one heteroatom being an N atom and with the N-atom of the piperidine ring adjacent to X², and
wherein the purine group in any of Formula (I), (II), (III), or (IV) is subject to tautomeric rearrangements;
and an amphiphilic micelle-forming agent.

In a second aspect, a micelle composition or pharmaceutical composition as defined herein is provided for use in the prevention, treatment or amelioration of a disease or disorder.

In a third aspect, a method for in vivo activation of immune cells in a subject is provided, comprising administering the micelle composition or pharmaceutical composition as defined herein to said subject in an amount sufficient to activate said immune cells.

In a fourth aspect, a method for enhancing or potentiating a treatment comprising radiotherapy and/or administration of a chemotherapeutic agent or an immune checkpoint inhibitor is provided, the method comprising administering the micelle composition or pharmaceutical composition as defined herein to said subject in combination with radiotherapy, administration of a chemotherapeutic agent and/or administration of an immune checkpoint inhibitor.

### DESCRIPTION OF DRAWINGS

FIG. 1: FIG. 1A: Overview of micelles used, micelle name, composition, molar ratio, surface charge (Zeta), size and Polydispersity Index (PDI). Micelles were composed of 1,2-dioleoyl-sn-glycero-3-phosphoethanolamine-N-[methoxy(polyethylene glycol)-2000] (DOPE-PEG2000 or PEG) and TLR7 agonist 1v270 (2-(4-((6-amino-2-(2-methoxyethoxy)-8-oxo-7H-purin-9(8H)-yl)methyl)benzamido)ethyl 2,3-bis(oleoyloxy)propyl phosphate) (1v270). Composition and molar ratio of each component in the micelles is shown. Micelle surface charge expressed in mV. Micelle size measured in nanometer (nm). Error is expressed as SEM. Micelle names are used in the figures to clarify exact composition. FIG. 1B: Micelle size by number distribution for the four micelles tested. Micelle size increases when 1v270 is added to the formulation. FIG. 1C: Micelle size by number distribution and zeta potential for the four micelles tested. Micelle size increase from 11.5 nm to 13-14 nm when 1V270 is added to the formulation. Zeta potential of the micelles decreases from −4 mV to −8 mV when 1V270 is added to the formulation.

FIG. 2: Structure of the formulated TLR7 agonist 1v270 (also termed TMX-201) used for micelle preparations (Mw=1085.4, Name: 2-(4-((6-amino-2-(2-methoxyethoxy)-8-oxo-7H-purin-9(8H)-yl)methyl)benzamido) ethyl 2,3-bis(oleoyloxy)propyl phosphate).

FIG. 3: FIG. 3A-B compares antitumor studies between micelle containing 1V270 and liposome containing 1V270. FIG. 3C shows antitumor activity of the micelle formulation MBS8 at three doses of 50, 200 and 400 nmol injected per mouse with first injection day 9, and then 4 more doses with 4 days interval. FIG. 3D shows a benefit of combination with a low dose MBS8 micelle at 50 nmol/mouse and optimal PD-1 treatment. FIG. 3E shows median survival in groups of mice treated with radiotherapy in combination with liposomes and micelles containing 1V270. FIG. 3F shows tumor to control ratio (average tumor size of groups of mice treated with micelles or liposomes with 1V270 in combination with radiotherapy (Tumor=T), compared to average tumor size of control radiotherapy treated groups (Control=C)). Calculations are made by T/Cx100. Micelles showed a significantly better anti-tumor activity than liposomes (p<0.01). FIG. 3G shows tumor growth inhibition calculated by subtracting 100-T/C ratio. Micelles showed a significantly better tumor growth inhibition than liposomes (p<0.01).

FIG. 4: FIG. 4A-G show cytokine secretion in plasma from mice treated with micelles or liposomes at equal doses. In general liposome MBS1 showed higher cytokine secretion than micelles for all cytokines measured, and MBS2 liposomes showed higher cytokine secretion than micelles for most cytokines tested. This indicates that micelles have a reduced risk of Cytokine Related Syndrome (CRS) compared to liposomes containing 1V270.

FIG. 5: FIG. 5A-B show induction of anti PEG IgM and IgG molecules in plasma of mice treated once with IV injection of micelles or liposomes containing 1V270. The anti PEG IgM and IgG molecules are measured by ELISA designed for this purpose. FIG. 5C show specificity of anti-PEG IgM determined with a competition assay. Only PEGylated liposomes are recognized by the generated IgM, whereas free PEG chains, DSPE-PEG micelles and MBS8 micelles are not recognized by the anti-PEG IgM molecules.

FIG. 6: Tumor growth and survival of mice bearing subcutaneous CT26 tumors and treated with 50, 100, or 200 nmol micelles in combination with radiotherapy (RT). Mice were treated on day 12 post inoculation and treated on 5 consecutive days with 2 Gy RT (day 12,13,14,15,16) and every fourth day for a total of 5 treatments with micelles (day 12,16, 20, 24, 28). Groups contain 8-10 mice. Tumor growth curves are displayed as mean±SEM. Statistical significance on survival was determined using Mantel-Cox test. FIG. 6A: Tumor growth of mice treated with MBS6 in combination with radiotherapy. FIG. 6B: Survival of mice treated with MBS6 in combination with radiotherapy. FIG. 6C: Tumor growth of mice treated with MBS7 in combination with radiotherapy. FIG. 6D: Survival of mice treated with MBS7 in combination with radiotherapy. FIG. 6E: Tumor growth of mice treated with MBS8 in combination with radiotherapy. FIG. 6F: Survival of mice treated with MBS8 in combination with radiotherapy.

FIG. 7: Compares antitumor activity between MBS8 micelles containing DOPE-PEG2000 and TLR7 agonist 1v270 in a 90:10 molar ratio and dosed at three levels in the CT26 model. MBS8 was dosed at three doses of 50 (small black circle), 200 (grey circle) and 400 (large black circle) nmol injected IV per mouse. The two high doses at 200 and 400 nmol were significantly different from the control treated group (p<0.001). Individual growth of each tumor is shown in FIG. 7B with 1) control treatment (PBS), 2) low dose MBS8 at 50 nmol/injection/mouse 3) mid dose MBS8 at 200 nmol/injection/mouse and 4) high dose MBS8 at 400 nmol/injection/mouse.

FIG. 8: MBS8 micelles at three different dose levels were combined with αPD-1 treatment (clone RMP1-14 with 10 mg/kg dosed IP) in the CT26 colon cancer syngenic mouse tumor model. MBS8 was dosed at three doses of 50, 200 and 400 nmol injected IV per mouse with first injection day 9, and then 4 more doses with 4 days interval at days 13, 17, 21 and 25. αPD-1 mAbs were injected IP starting day 11, twice weekly for three weeks. PD-1-MBS8 combination treatments were all significantly stronger than PD-1 monotherapy (p<0.05, p<0.005, Wilcoxon rank sum test) (FIG. 8A). In FIG. 8B is shown individual tumor growth for all 10 mice per group with 1) control (PBS treated), 2) MBS8 at 50 nmol, 3) MBS8 at 200 nmol, 4) MBS8 at 400 nmol 5) PD1 treatment alone at 10 mg/kg injected IP 6) PD-1 and MBS8 at 50 nmol in combo therapy, 7) PD-1 and MBS8 at 200 nmol in combo therapy 8) PD-1 and MBS8 at 400 nmol in combo therapy.

FIG. 9: MBS8 potentiates anti-tumor effect of doxorubicin (A-B) and doxil (C-D). Mean tumor volumes±SEM are shown, n=10 in (A) and (C). Wilcoxon rank sum test was used for statistical data treatment. Tumor volumes of individual animals are shown for the indicated groups in (B) and (D).

FIG. 10: Tumor growth of mice bearing subcutaneous CT26 tumors treated with radiotherapy (RT) in combination with 100 nmol MBS8 intratumorally or 200 nmol MBS8 intravenously. When indicated, mice were treated with lipid matched vehicle instead of MBS8. Mice were treated on day 12 post inoculation and treated on 5 consecutive days with 2 Gy RT and every fourth day for a total of 5 treatments with micelles. Groups contain 8 mice. Tumor growth curves are displayed as mean±SEM. FIG. 10A: Tumor growth of mice treated with MBS8 intratumorally or intravenously in combination with radiotherapy. FIG. 10B: Tumor growth of mice treated with vehicle intratumorally or intravenously in combination with radiotherapy. FIG. 10C: shows survival of mice treated with MBS8 by the intratumoral route either micelles without the TLR7 agonist (vehicle) or MBS8 with the TLR7 agonist 1V270. Groups are shown either with or without combination with radiotherapy. FIG. 10D: shows survival of mice treated with MBS8 by the intravenous route either micelles without the TLR7 agonist (vehicle) or MBS8 with the TLR7 agonist 1V270. Groups are shown either with or without combination with radiotherapy. The group treated with RT+iv MBS8 showed significantly better anti-tumor activity than RT alone (p=0.015), whereas the group treated with RT+it MBS8 did not show a significantly better anti-tumor activity than RT alone, n=8/group.

FIG. 11: Tumor growth and survival of mice bearing subcutaneous EL4 or MC38 tumors treated with radiotherapy (RT) in combination with 200 nmol MBS8 or vehicle. Tumor growth curves are displayed as mean±SEM.

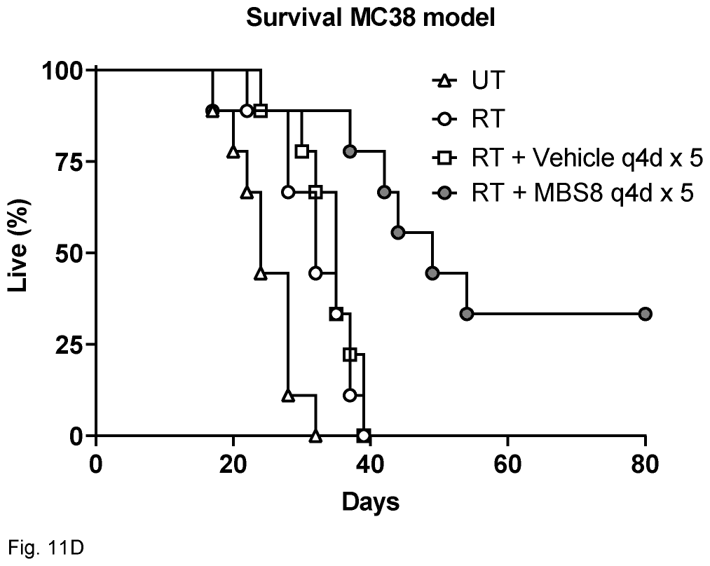

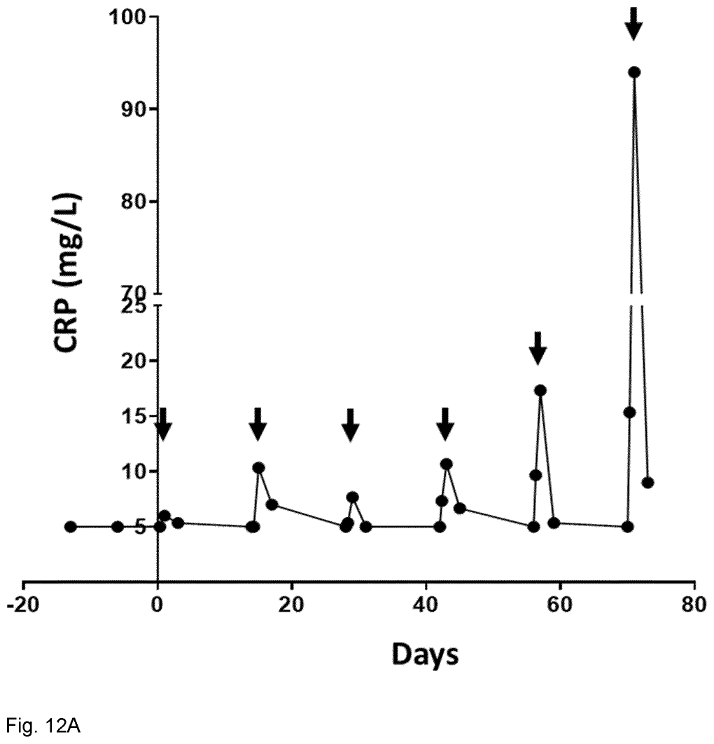

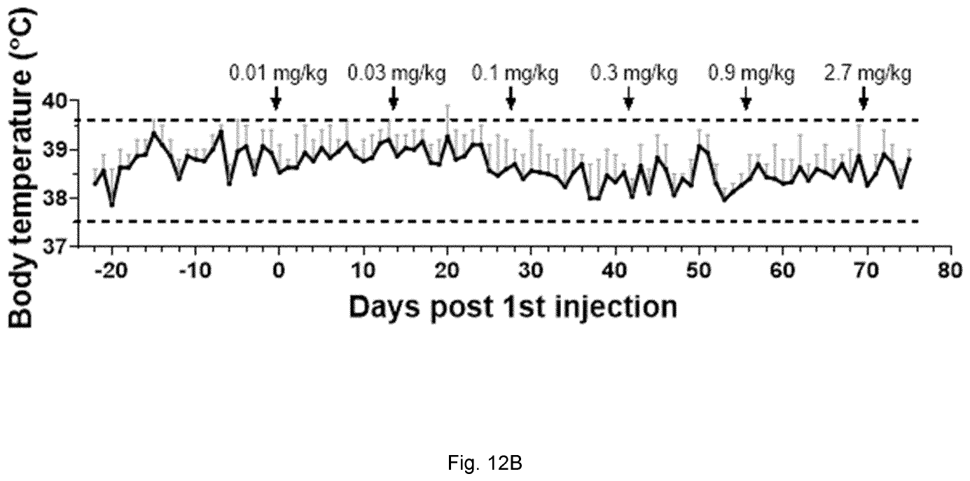

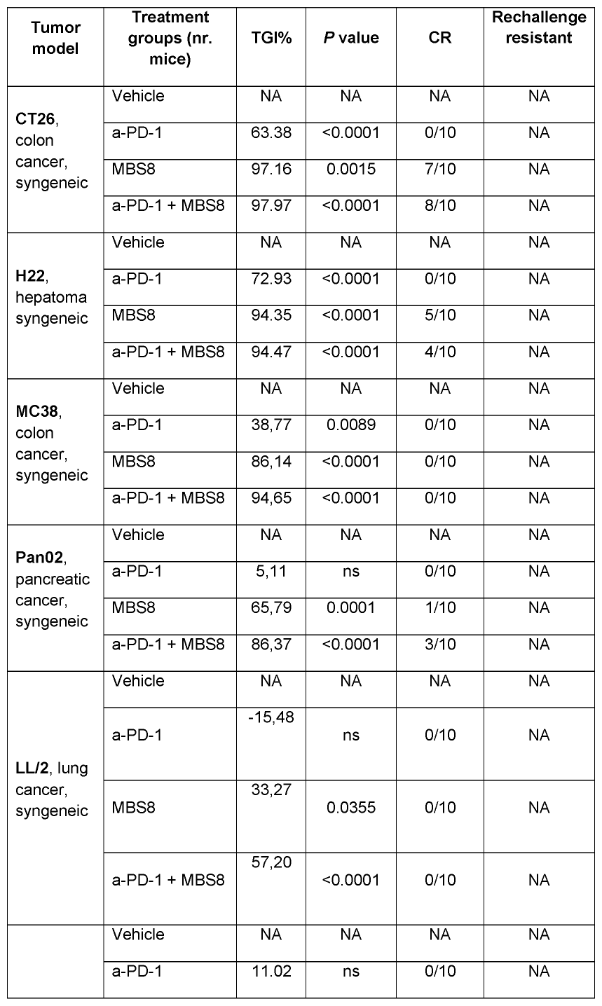

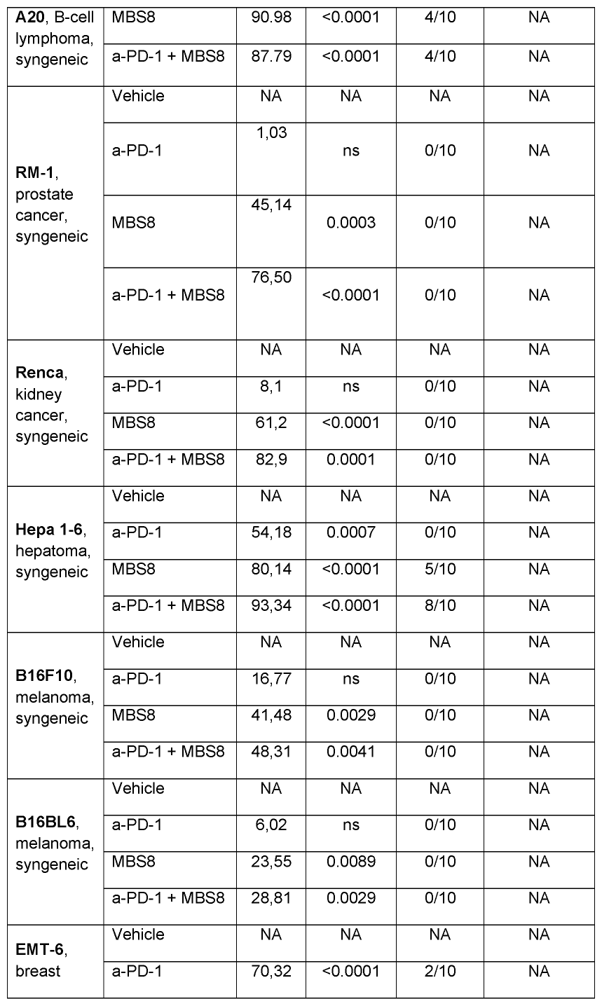

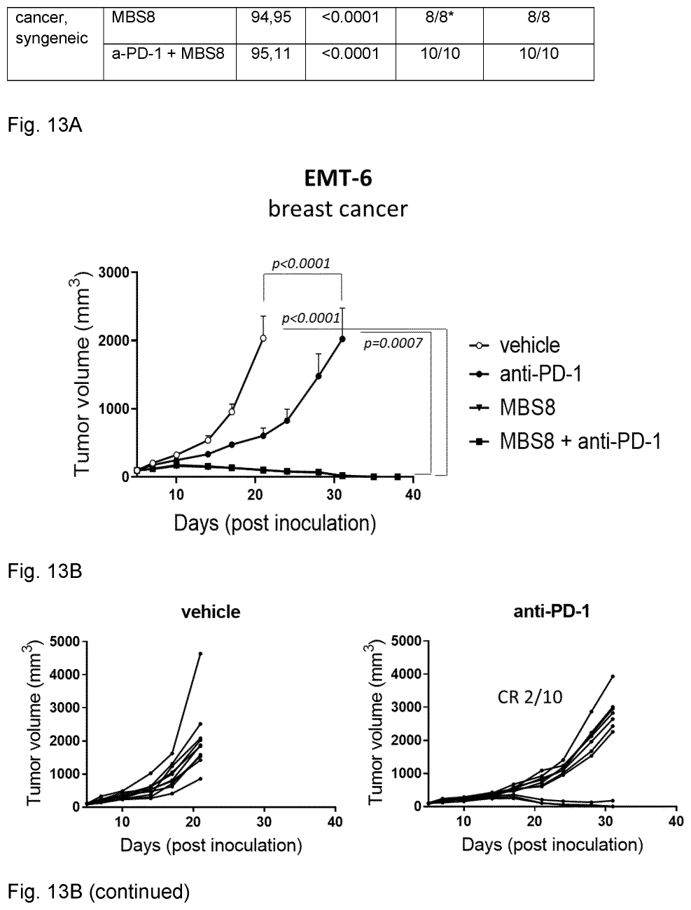

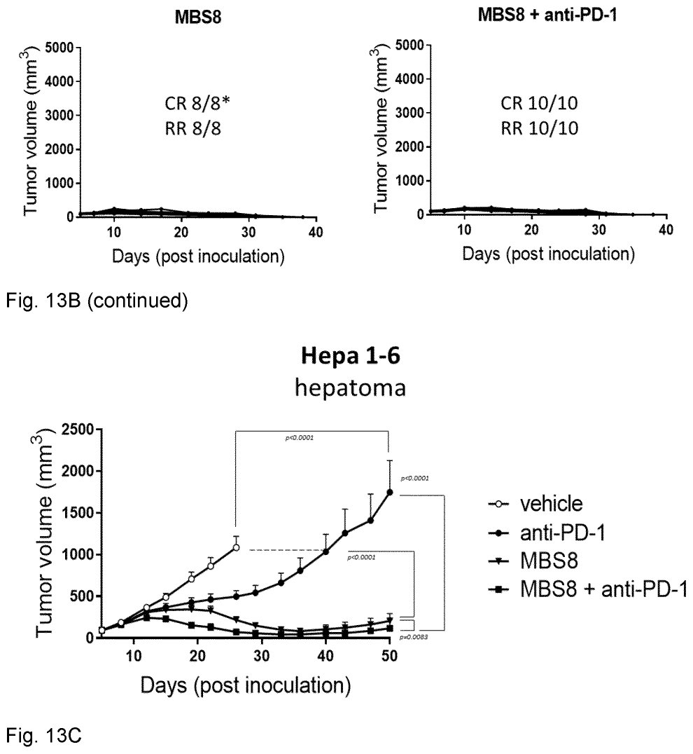

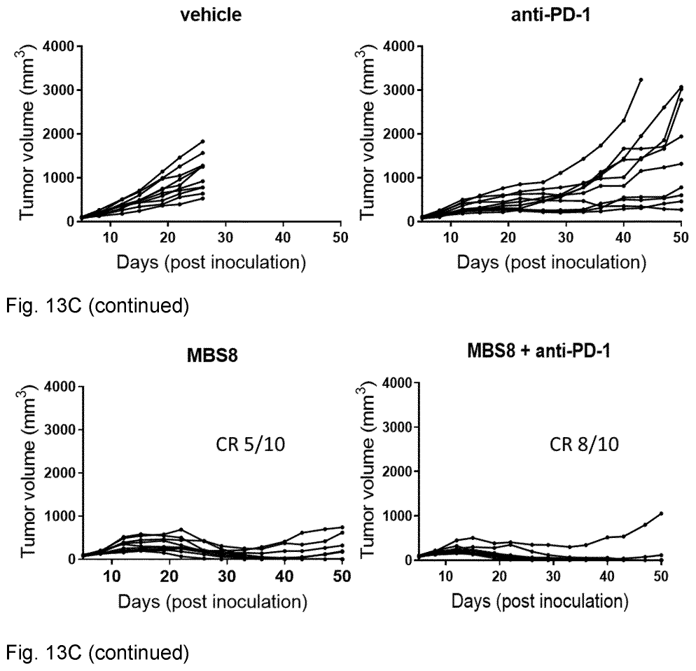

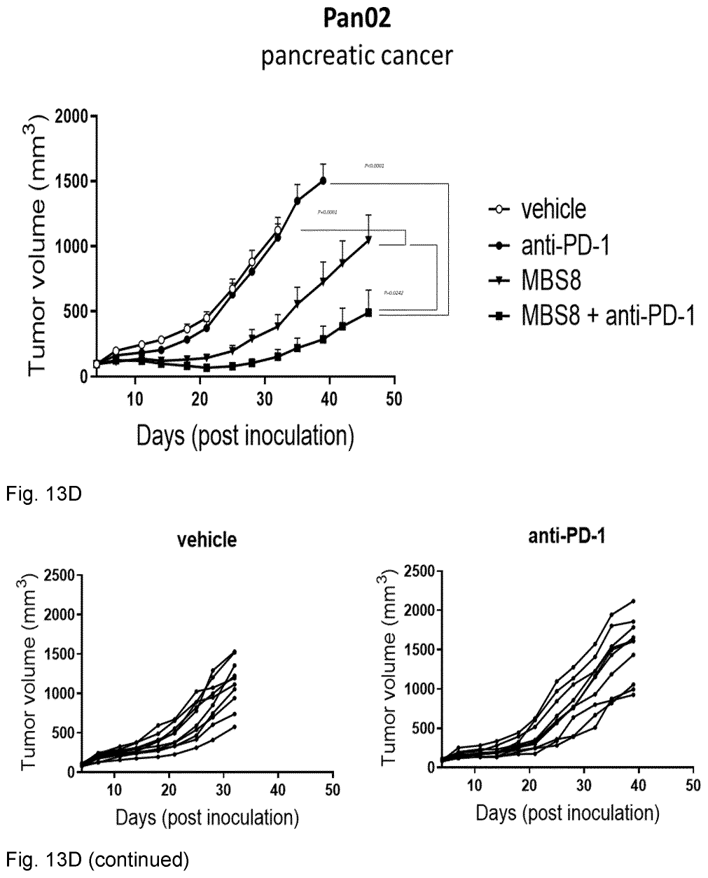

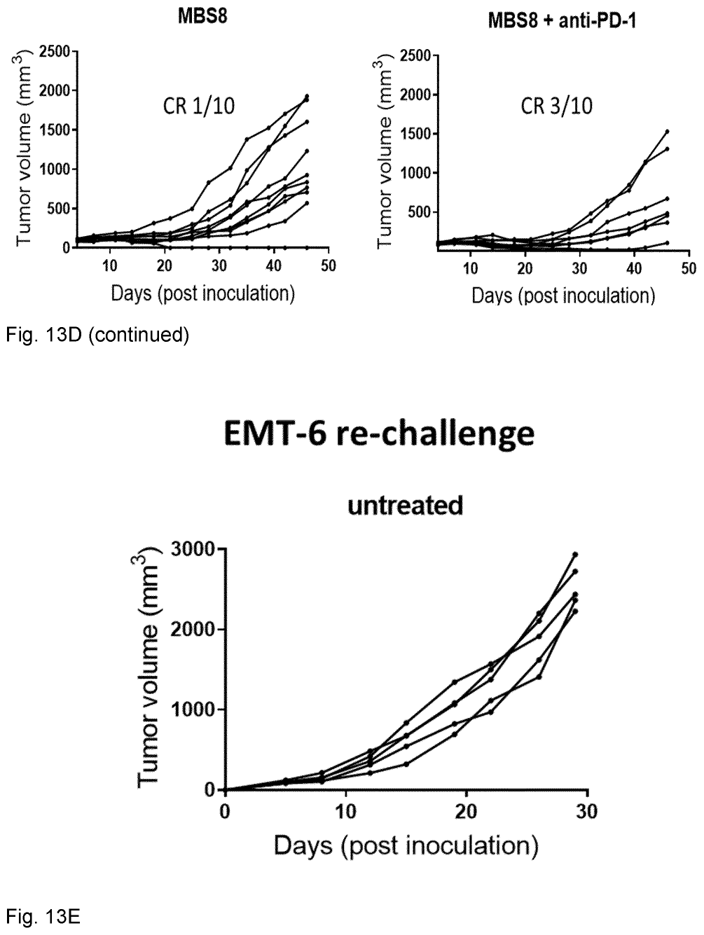

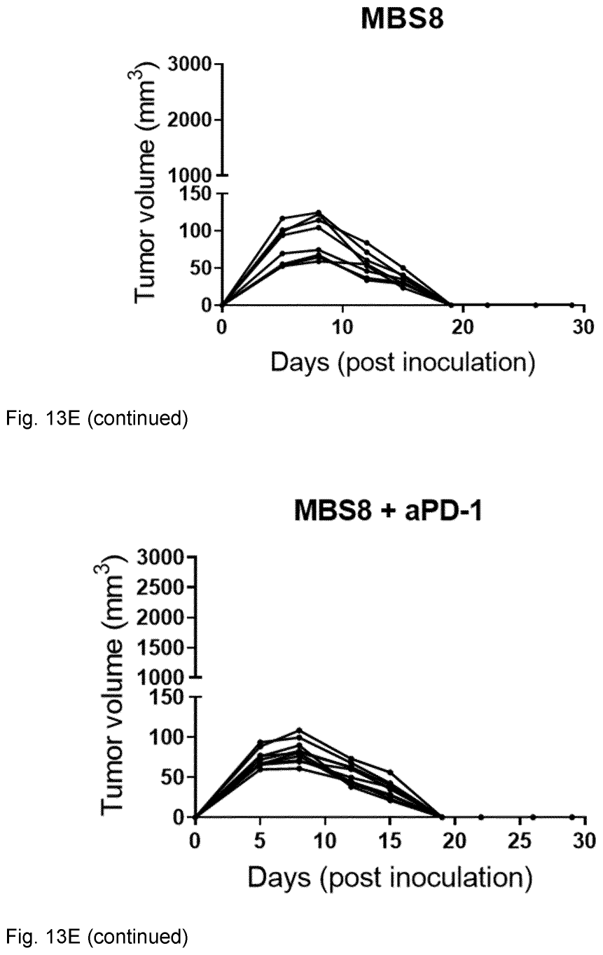

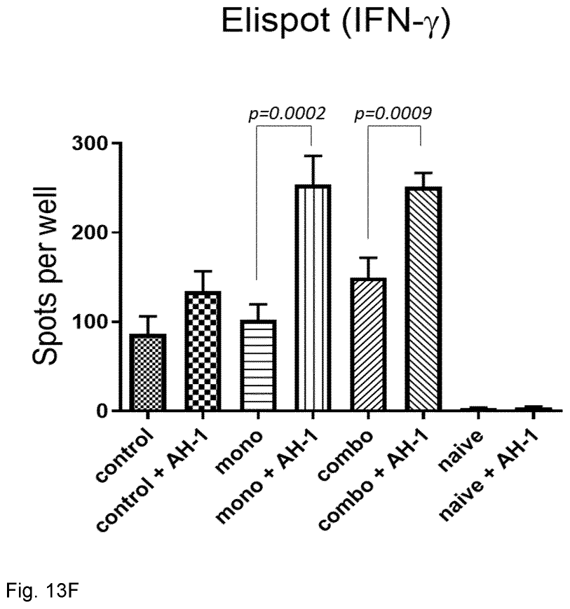

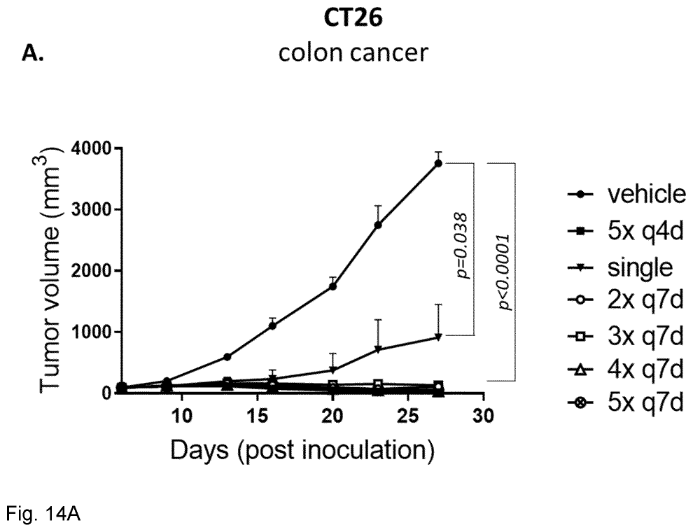

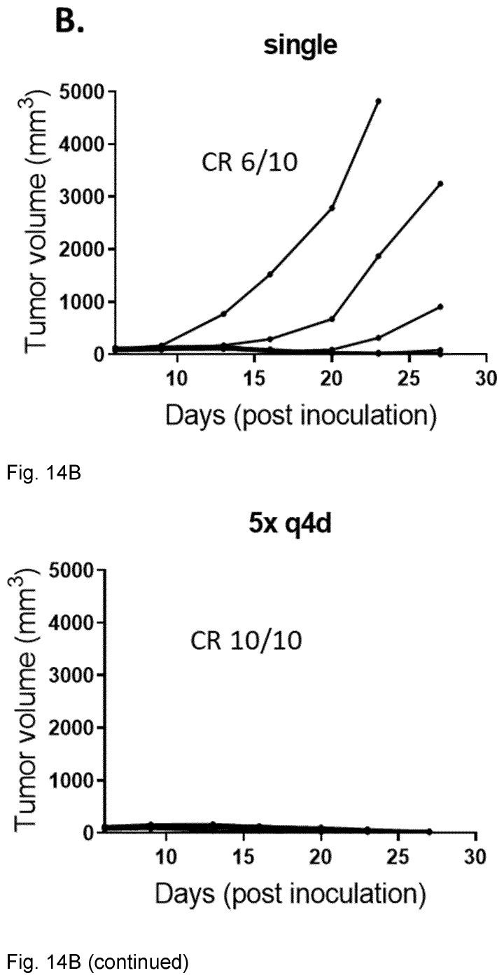

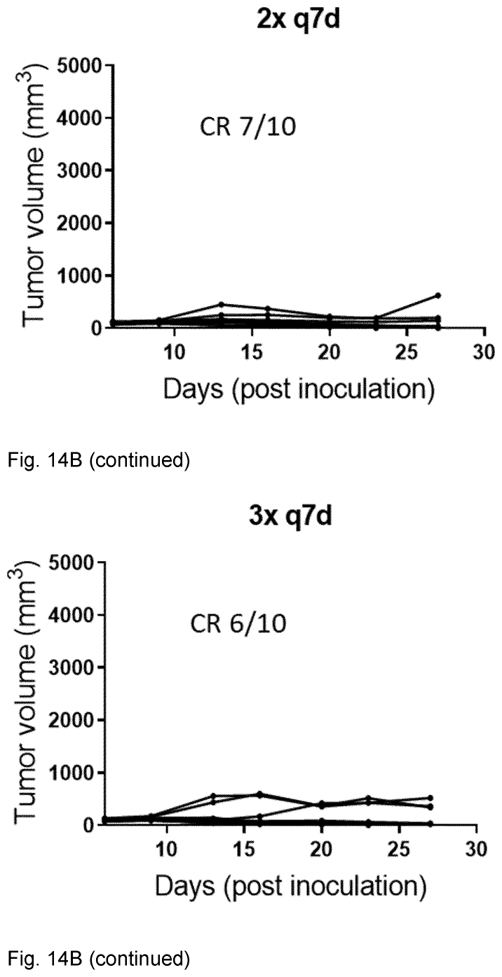

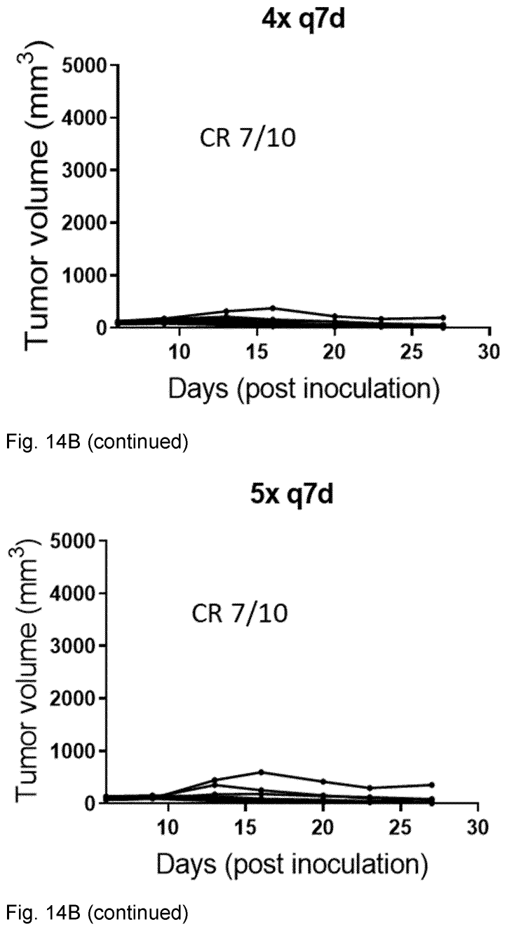

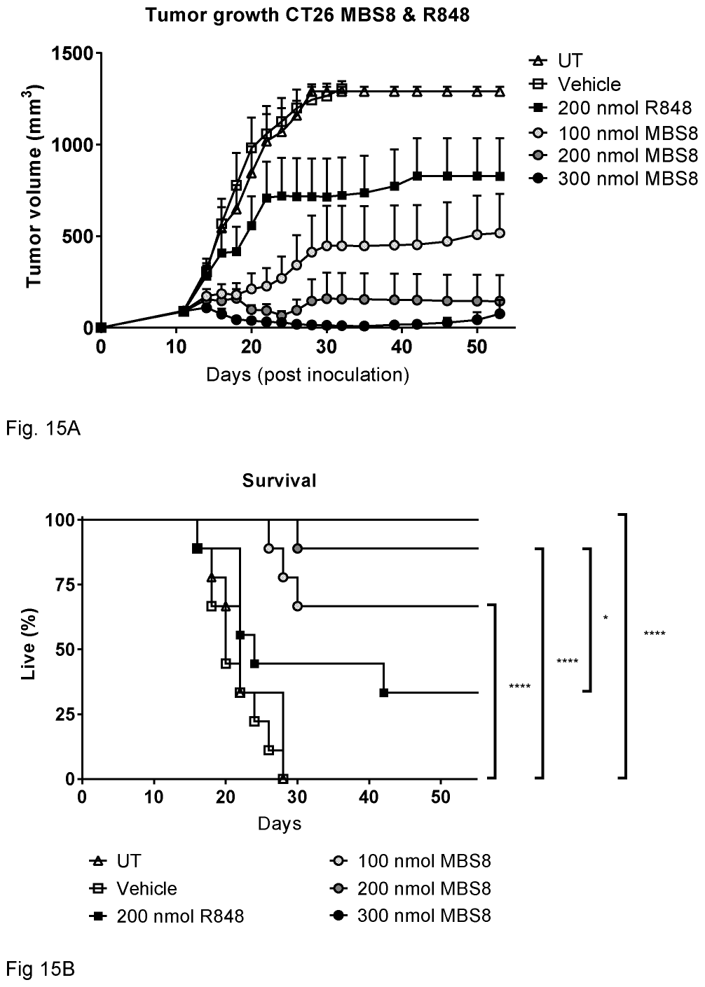

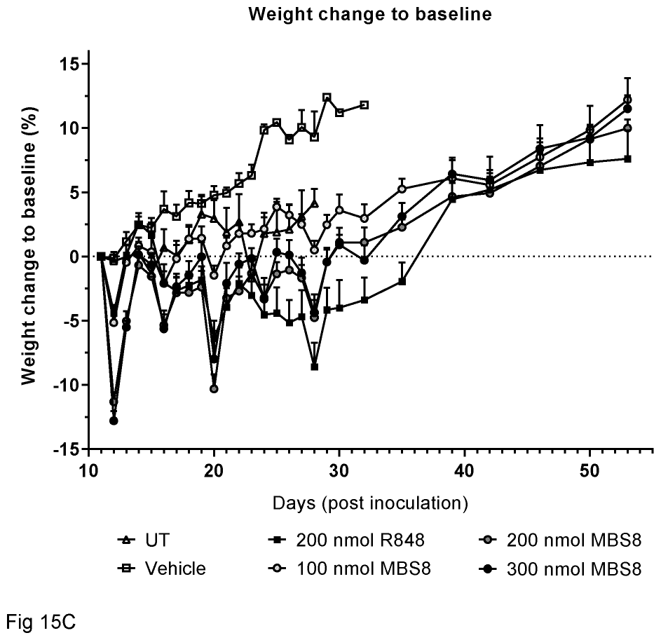

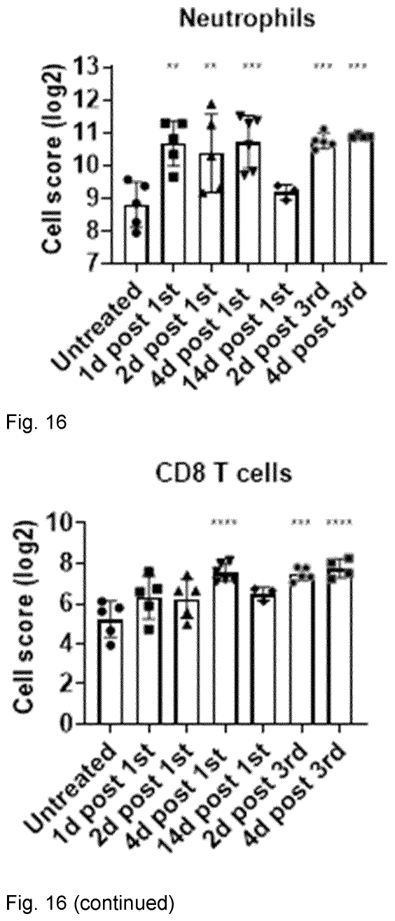

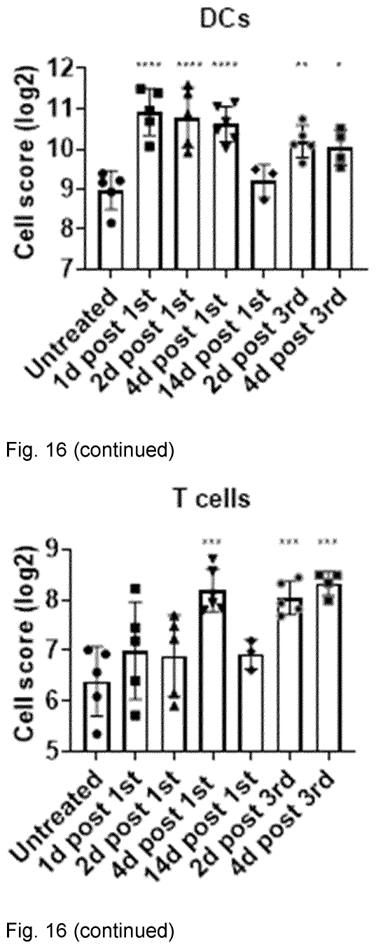

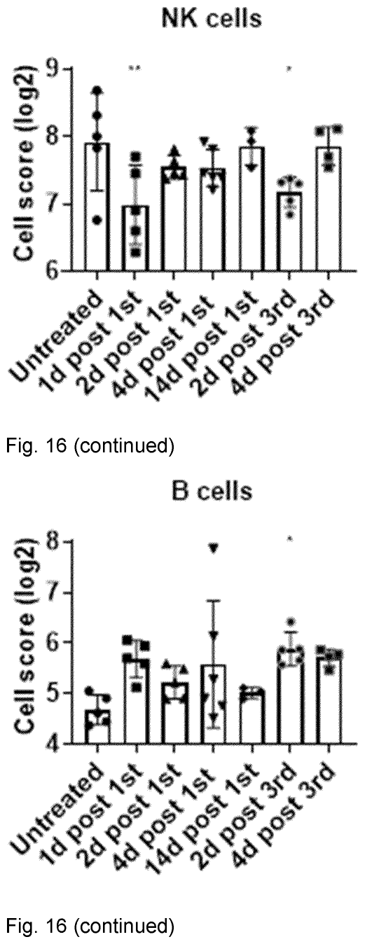

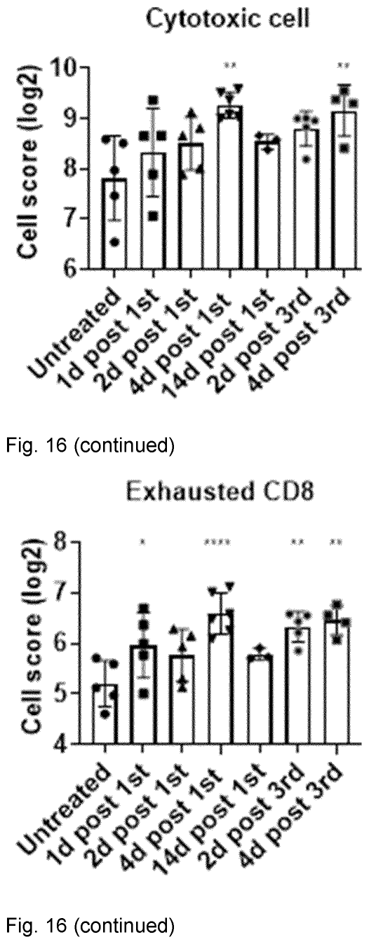

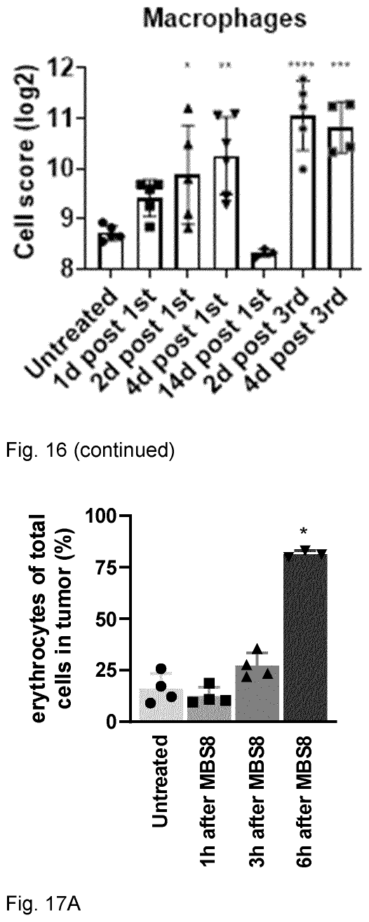

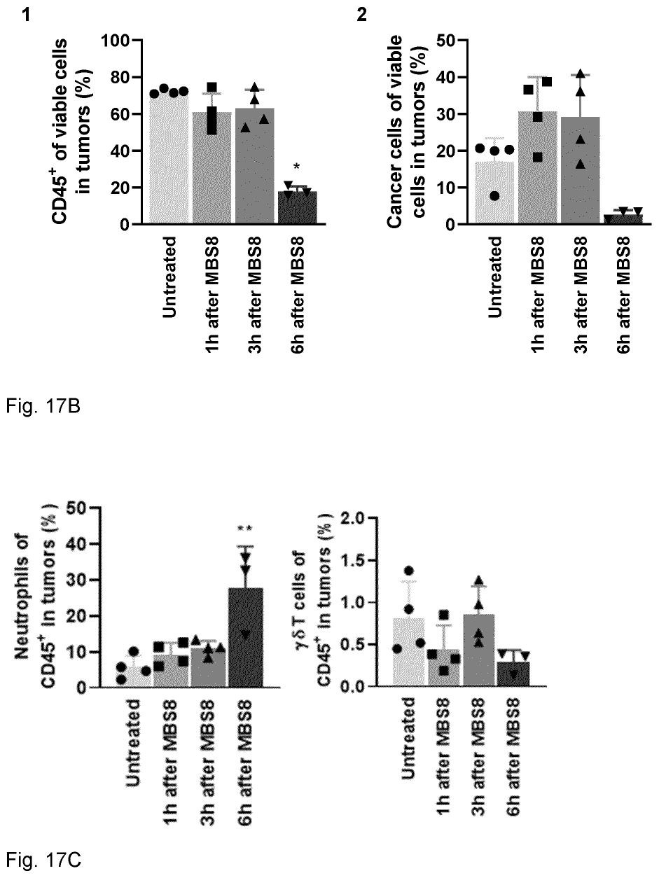

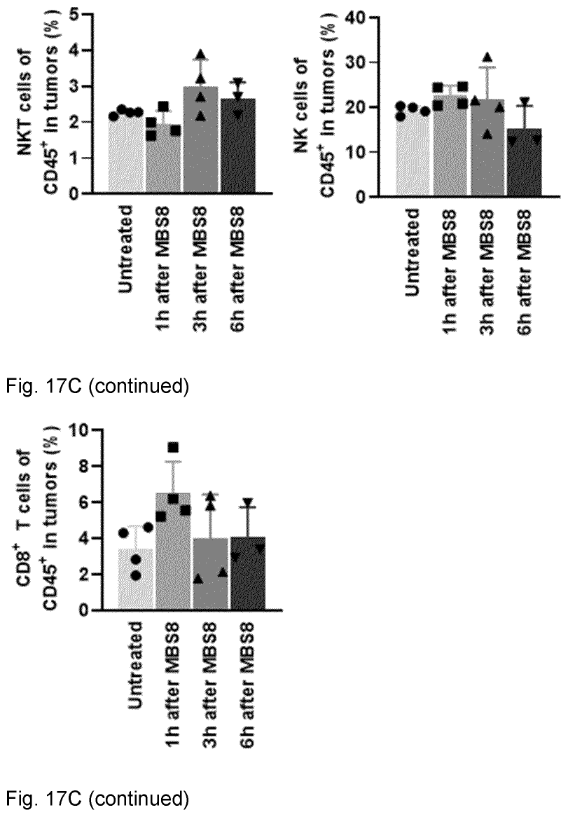

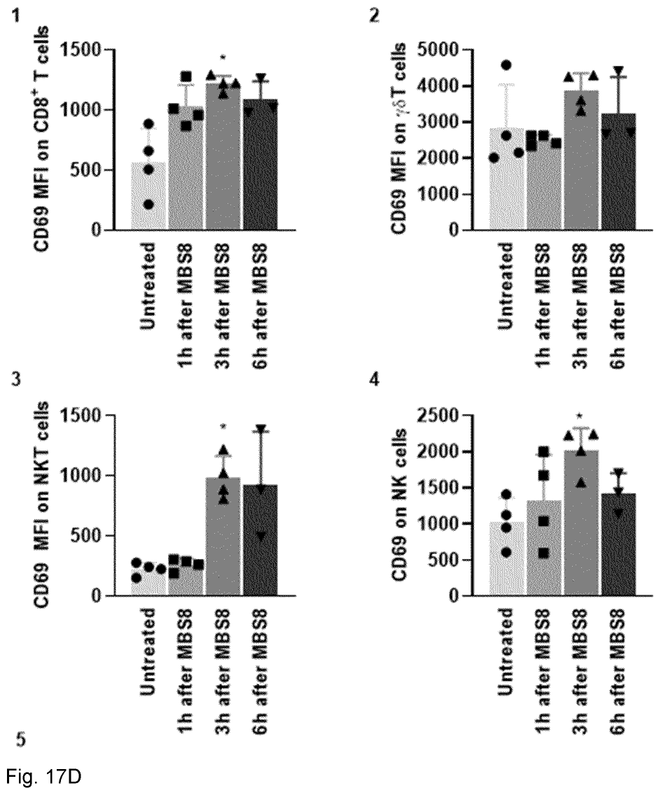

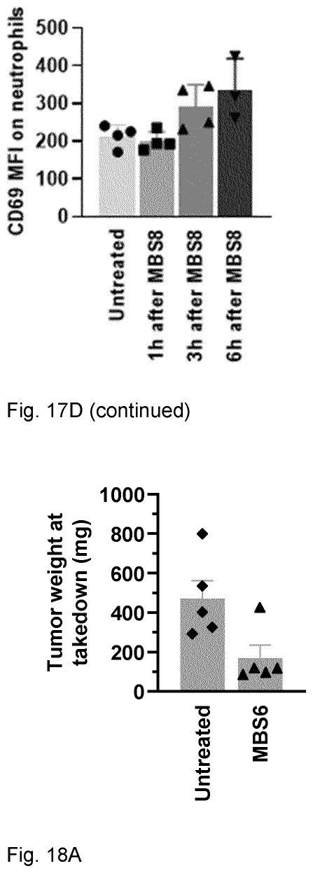

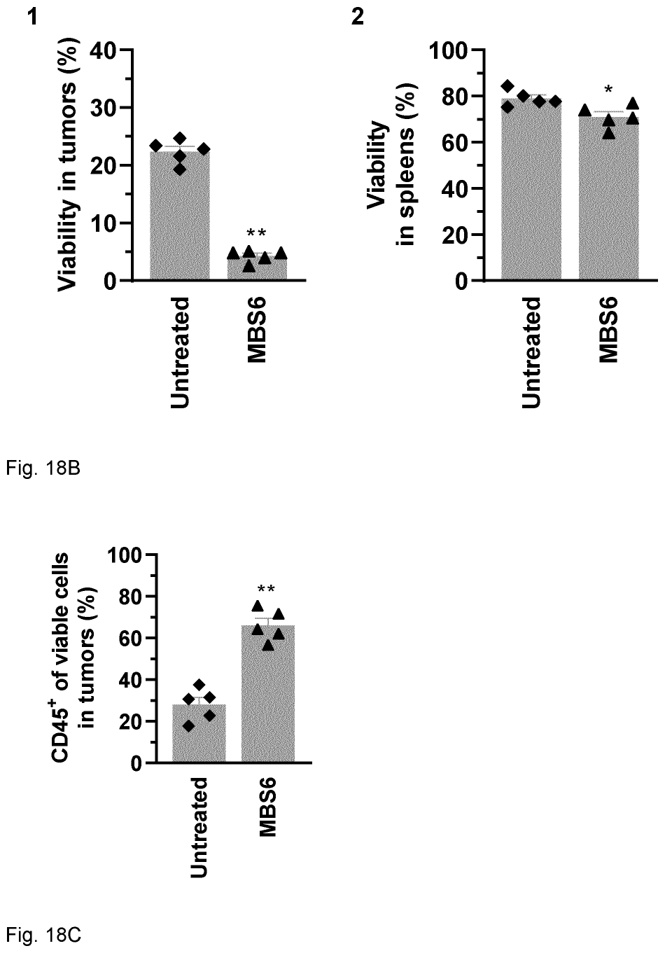

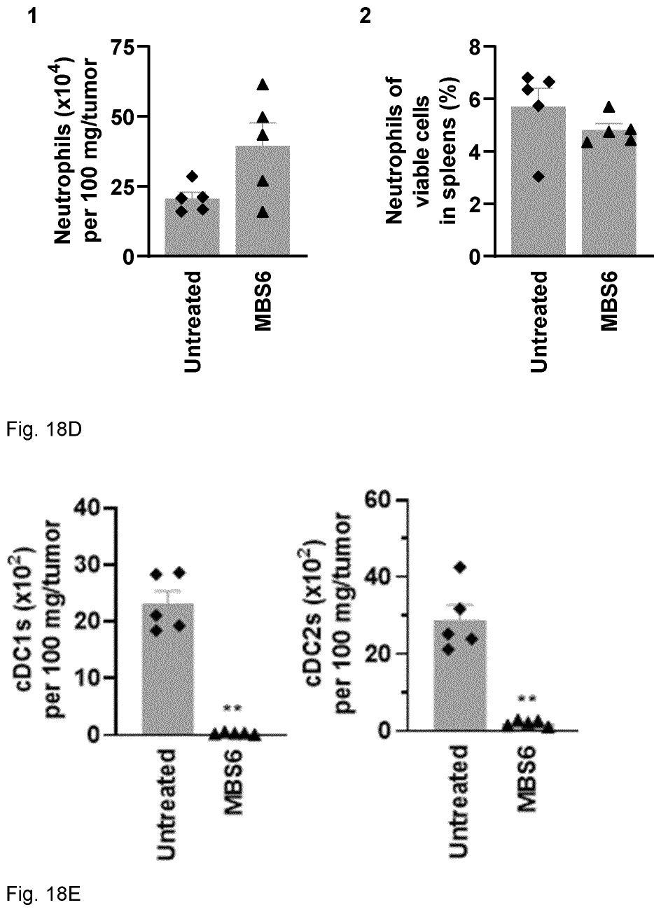

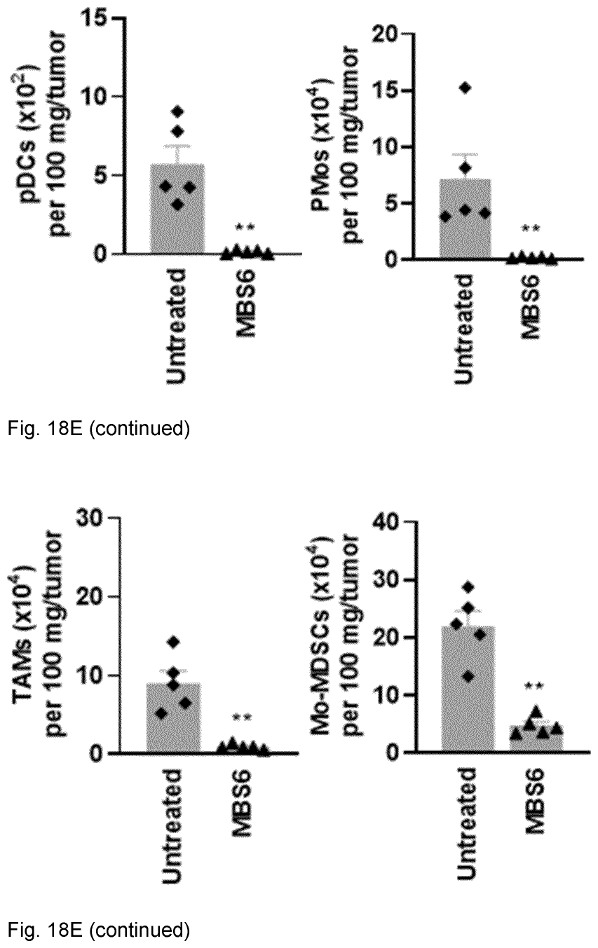

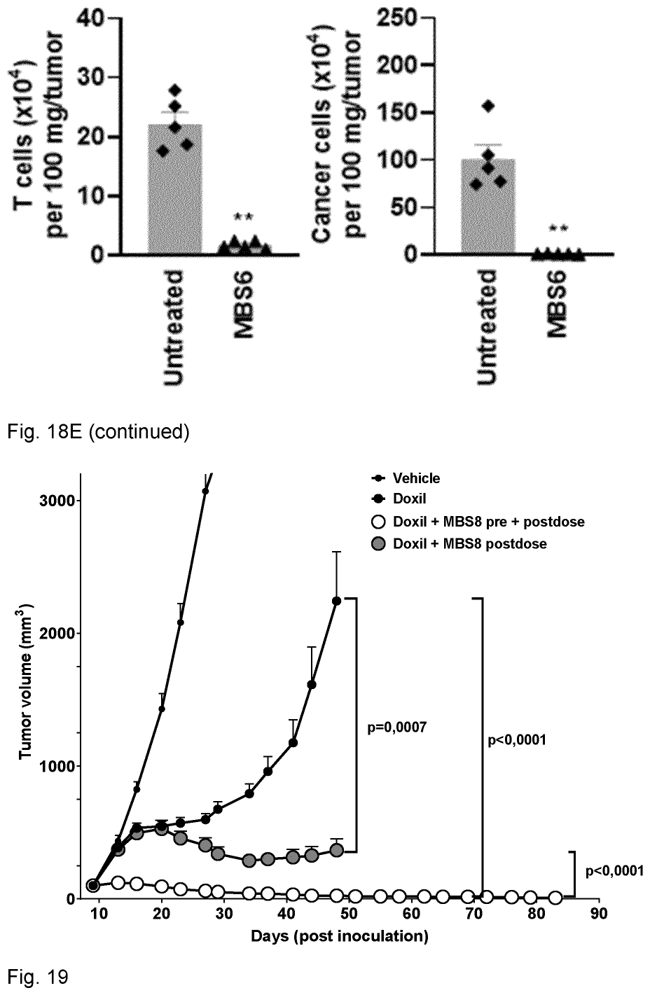

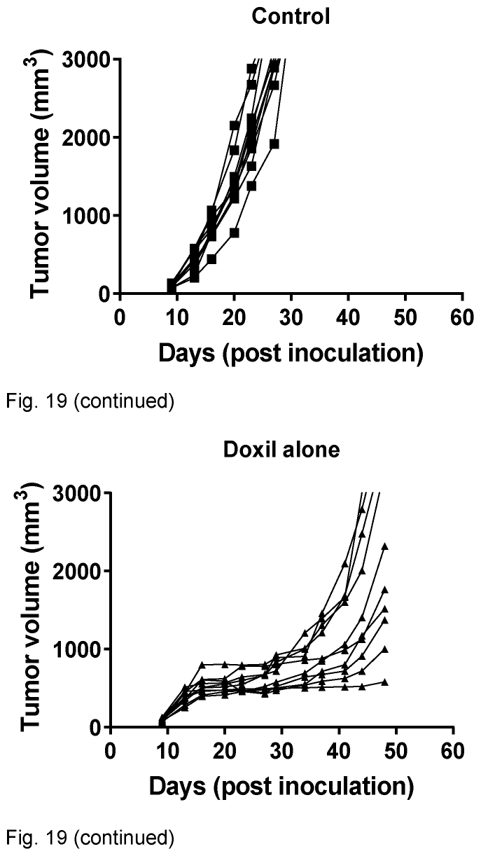

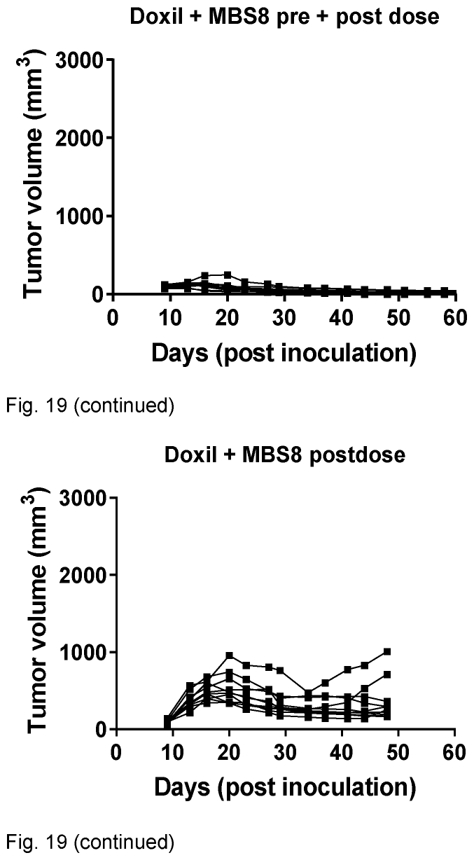

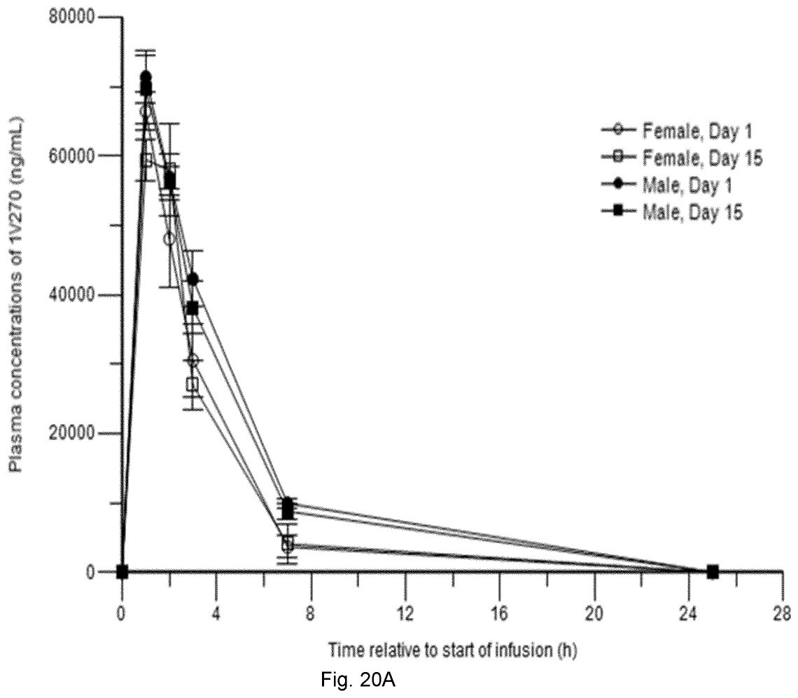

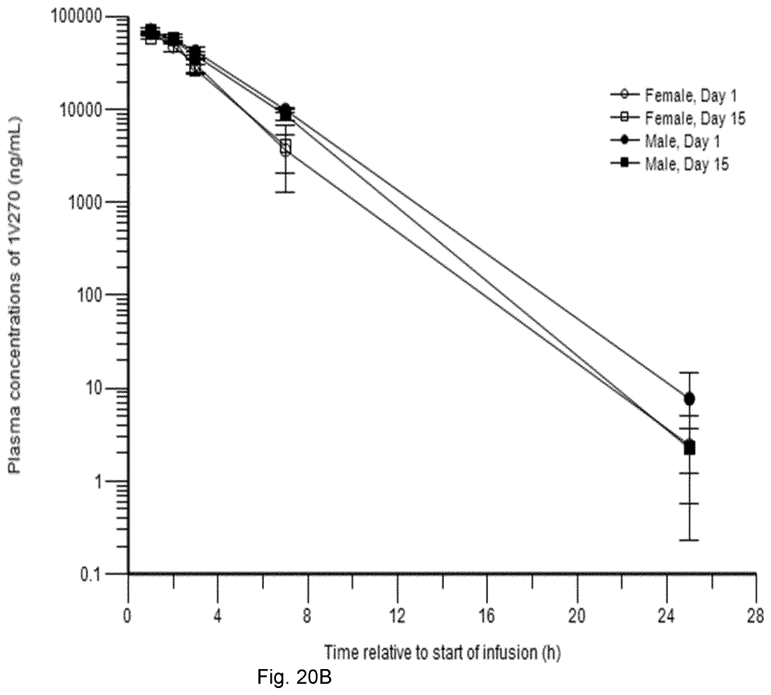

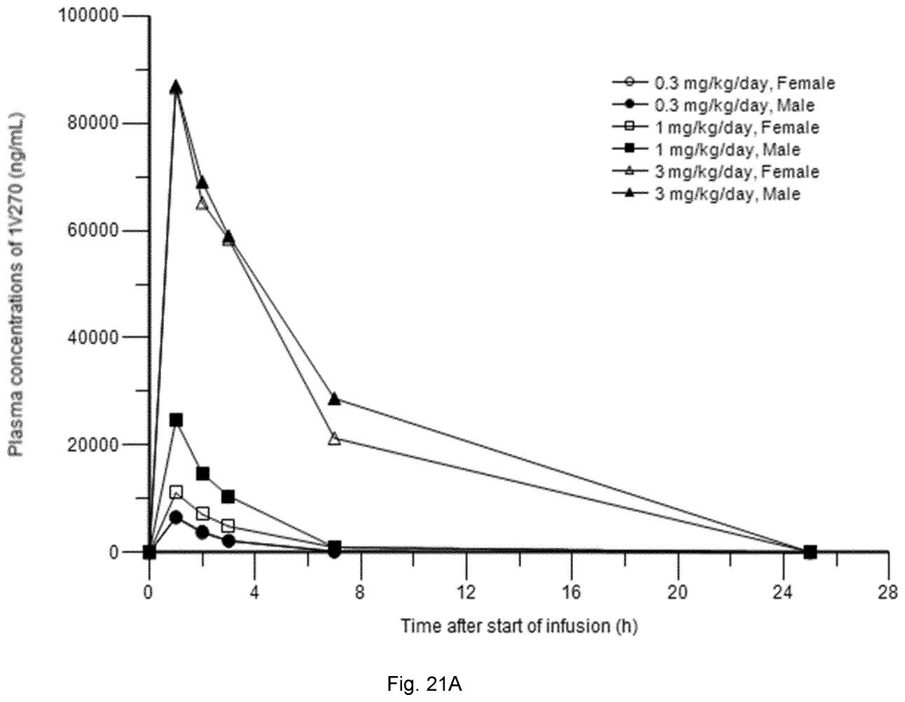

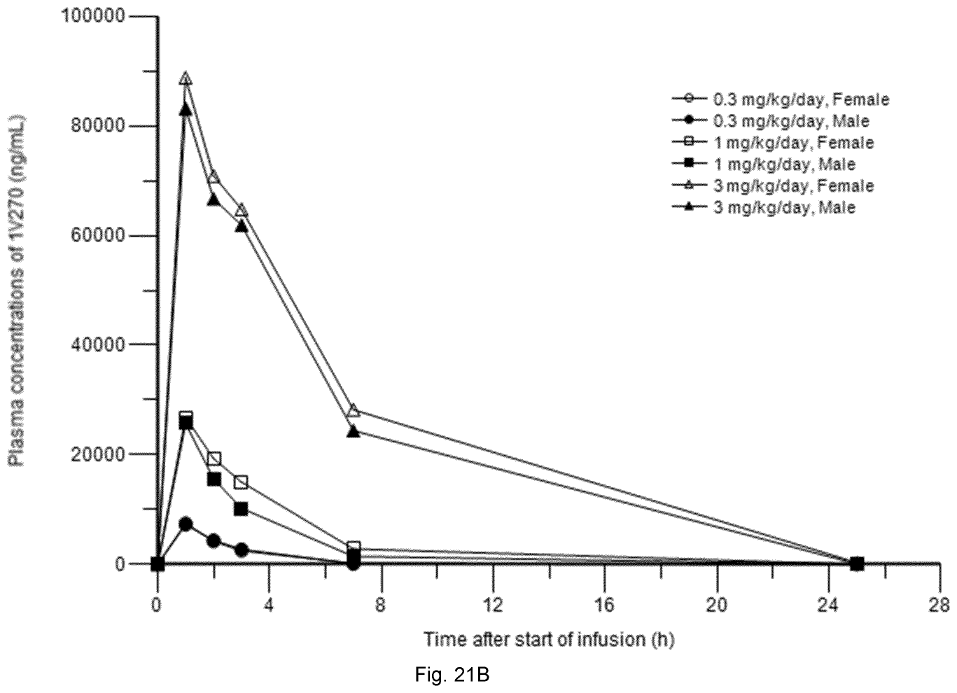

EL4 bearing mice started treatment on day 7 after inoculation and were treated with 2 Gy RT on 3 consecutive days and MBS8 or vehicle every fourth day for a total of 5 treatments. Groups contain 9-10 mice. FIG. 11A: Tumor growth of treated EL4 bearing mice. FIG. 11B: Survival of treated EL4 bearing mice.

MC38 bearing mice started treatment on day 10 after inoculation and were treated with 2 Gy RT on 5 consecutive days and MBS8 or vehicle every second (q2d), fourth (q4d), seventh day (q7d) for a total of 5 treatments. Groups contain 9 mice. FIG. 11C: Tumor growth of treated MC38 bearing mice. FIG. 11D: Survival of treated MC38 bearing mice.

FIG. 12: MBS8 administration in cynomolgus monkeys. (A) C-reactive protein (CRP) was tested immediately before drug administration and at 8, 24 and 72 h post-infusion. Data represent mean values (n=3). The arrows indicate the peak of CRP at 24 h after each injection. Respective doses are as indicated in FIG. 12B. (B) Body temperature was monitored daily. Mean+SD values (n=3) are shown. Days of drug administration and respective doses are indicated. Dashed line indicates normal range based on the baseline measurements during acclimation period (Day −22-Day 0).

FIG. 13 In vivo efficacy studies using 12 syngeneic subcutaneous tumor models. (A) Table summarising results for all 12 models. Mice were randomized when average tumor size reached ˜100 mm³ and treated with the indicated remedies. αPD-1 (clone RMP1-14) was administered at 10 mg/kg i.p. six times at q4d, and MBS8 was dosed at 300 μg/mouse i.v., five times at q4d. The same dosing regimens were used for the combination studies. All groups included 10 mice/group. TGI, tumor growth inhibition; CR, complete responders; NA, not applicable. (B-D) Effect of MBS8 on the growth of subcutaneous tumors. Top panels: tumor growth curves in the indicated groups of animals. Mean tumor volumes+SEM are shown. Significant two-sided p-values are shown (Wilcoxon rank sum test). Middle and lower panels: tumor growth curves of individual animals in the indicated treatment groups are shown. (B) EMT-6, (C) Hepa 1-6 and (D) Pan02 are representative of three different response patterns found in the 12 tumor panel. CR, complete responders; RR, re-challenge resistant; *two animals died at the beginning of treatment and were excluded from statistical analysis. (E) Rejection of re-challenged EMT-6 tumors. Mice which showed CR to MBS8 (n=8) or MBS8+ anti-PD-1 (n=10) treatment in the EMT-6 model and were tumor-free for at least three weeks were re-challenged on the contralateral flank with EMT-6 cells and were monitored for tumor growth 29 days. Naïve mice (n=5) innoculated with EMT-6 cells were used as untreated control. (F) Immune memory response in mice treated with MBS8 alone or in combination with a-PD-1. CR from the CT26 model were re-challenged and all demonstrated rejection of re-challenged tumors. In these animals, the presence of tumor specific T-cells was assessed using ELISPOT. Analysis was performed on splenocytes from untreated CT26 tumor bearing mice (control), mice previously treated with MBS8 and resistant to re-challenge (mono), mice treated with MBS8+a-PD-1 combo (combo) and naïve tumor-free mice (naïve) stimulated (+AH-1) or not with a CT26 tumor specific antigen AH-1 in vitro. Number of IFN-γ expressing cells per well was quantified. Means+SEM are shown; n=5 mice/group. Statistical analysis was performed using Wilcoxon rank sum test. Significant two-sided p-values are shown.

FIG. 14 Comparative efficacy of MBS8 administered at different dose schedules. The CT26 subcutaneous model was used. Mice were randomized when average tumor size reached ˜100 mm³ and treated with 200 nmol/mouse i.v. at the indicated dose schedules. (A) Mean tumor volumes+SEM are shown. Significant two-sided p-values are shown (two-tailed Wicoxon rank sum test). (B) Tumor growth curves of individual animals. Animals which tumors at the last measurement were <40 mm³ and showed continuous volume reduction are considered as complete responders (CR).

FIG. 15: MBS8 and R848 treatment of mice bearing subcutaneous CT26 tumors. CT26 tumor bearing mice started treatment on day 11 after inoculation and were treated intravenously with MBS8 (100, 200 or 300 nmol) or R848 (200 nmol) in a q4d schedule for a total of 5 treatments. n=9 mice/group. (A) Mean tumor growth curves±SEM are displayed. (B) Survival of treated CT26 bearing mice. Statistical significance on survival was determined using Mantel-Cox test, ****p<0.0001, *p<0.05 (C) Weight change to baseline, mean weight±SEM is displayed.

FIG. 16: Cell types in tumors from mice treated with MBS8 monotherapy based on gene expression analysis. Mice were treated with 200 nmol MBS8 in a q4d schedule for a total of 1 or 3 injections. Tumors were collected on day 0 (Untreated), 1 day post 1^{st} injection, 2 days post 1^{st} injection, 4 days post 1^{st} injection, 14 days post 1^{st} injection, 2 days post 3^{rd} injection, 4 days post 3^{rd} injection, n=3-6 for all groups. Gene expression analysis was performed on extracted RNA and cell type analysis was performed from the data with the Nanostring advanced software module. Two-way ANOVA with multiple comparison correction was performed to compare the different time points to untreated. * indicates p<0.05; ** indicates p<0.01; *** indicates p<0.001 and **** indicates p<0.0001

FIG. 17: Effects on the tumor microenvironment shortly after injection of MBS8 based on flow cytometric analysis. CT26-bearing mice were injected with 200 nmol MBS8 and tumors evaluated by flow cytometry. Tumors were collected 1, 3 and 6 hours post injection. All graphs are displayed as mean±SEM. Statistical significance compared to untreated tumors was determined using Kruskal-Wallis test with Dunn's multiple comparisons tests where * indicates p<0.05; ** indicates p<0.01; and *** indicates p<0.001. n=3-4. MFI=Median fluorescent intensity.

FIG. 18: Effects on the tumor microenvironment during treatment based on flow cytometric analysis. Mice bearing CT26 tumors were treated on day 15 after cancer cell inoculation with 200 nmol MBS6 every fourth day and tumors and spleens analyzed two days after the second injection. All graphs are displayed as mean±SEM. Statistical significance compared to untreated tumors was determined using Mann-Whitney U test where * indicates p<0.05; ** indicates p<0.01; n=5.

FIG. 19: Dosing schedule study in CT26 tumor bearing mice with Doxil treatment for all groups were done at day 9, 13 and 17 (black circles), the Doxil+MBS8 pre+postdose treatment group was dosed additionally with MBS8 at day 9 and day 19, 23, 27 and 31, and the Doxil+MBS8 postdose treatment group was dosed additionally with MBS8 at day 19, 23, 27 and 31. All graphs are displayed as mean+SEM. Statistical significance compared between indicated groups was determined using Mann-Whitney U test at indicated significance levels, n=10.

FIG. 20: Mean (+/−SD) plasma concentration-time curves of MBS8 in female and male rats on Day 1 (first dose) and Day 15 (one week after last dose of two doses 1 week apart) after intravenous infusion administration over a period of 1 hour of 3 mg/kg/day MBS8 (1V270) at a linear scale (A) and semi-logarithmic scale (B). This figure demonstrates indifferent kinetics of MBS8 administered after 1 day and 15 days, (third dose) respectively. This observation supports that the micelles of the present invention do not trigger accelerated blood clearance (ABC).

FIG. 21: Overlay of individual plasma concentration-time curves of 1V270 in 1 female and 1 male cynomolgus monkey on Day 1 (A) (first dose) and Day 15 (B) (one week after last dose of two doses 1 week apart) after intravenous infusion administration over a period of 1 hour of 0.3, 1 and 3 mg/kg/day MBS8 (1V270) at a linear scale. This figure demonstrates indifferent kinetics of MBS8 administered after 1 day (one dose) and 15 days (third dose after weekly dosing), respectively. This observation supports that the micelles of the present invention do not trigger accelerated blood clearance (ABC).

### DETAILED DESCRIPTION

### Definitions

A “lipid” as disclosed herein refers to a group of substances comprising at least one hydrophobic part, which by itself would be insoluble in water. Exemplary groups of lipids may without limitation be fatty acids, glycerolipids, glycerophospholipids, sphingolipids, sterol lipids, prenol lipids, saccharolipids, and polyketides.

The term “prophylaxis”, as used herein, refers to prevention of a disease or prevention of spreading of a disease.

The term “treatment”, as used herein, refers to the combating of a disease or disorder. “Treatment” or “treating,” as used herein, includes any desirable effect on the symptoms or pathology of a disease or condition as described herein, and may include even minimal changes or improvements in one or more measurable markers of the disease or condition being treated. “Treatment” or “treating” does not necessarily indicate complete eradication or cure of the disease or condition, or associated symptoms thereof.

The term “PEG” as used herein, refers to polyethylene glycol.

The term “amphiphilic micelle-forming agent” as used herein refers to an agent which is both “amphiphilic” i.e. possessing both hydrophilic (water-loving, polar) and lipophilic (fat-loving) properties while also being capable of forming micelles. The amphiphilic micelle-forming agent disclosed herein are capable of forming micelles in a mixture with other compounds in solution. The amphiphilic micelle-forming agent described herein can also form other types of nanoparticles. In some embodiments, one or more amphiphilic micelle-forming agents can be used to form a nanoparticle such as a micelle, a liposome, a nanodisc, a lipoplex, or a lipidoid. The amphiphilic micelle-forming agent can also be used to form an emulsion with the TLR7 agonist of the present disclosure.

The term “linking group” as used herein refers to a grouping of bonded atoms, such as a functional group, which covalently connects at least two parts of a molecule. In one example, a linking group is a carbonyl group, i.e. “—C(O)—” that may on one end bind e.g. an amine “H2N-alkyl(1)” and on the other end bind an alkyl(2) thereby forming an amide “alkyl(2)-C(O)—NH-alkyl(1)”.

The terms “anti-PD-1”, “α-PD-1, and “α-PD-1” are used interchangeably herein to refer to the group of immune check point inhibitors, anti-PD-1.

The terms “anti-PD-L1”, “α-PD-L1, and “a-PD-L1” are used interchangeably herein to refer to the group of immune check point inhibitors, anti-PD-L1.

The term “MBS6” as used herein, refers to a micelle composition of DSPE-PEG2000:1V270 in a molar ratio of 80:20.

The term “MBS7” as used herein, refers to a micelle composition of DSPE-PEG2000:1V270 in a molar ratio of 95:5.

The term “MBS8” as used herein, refers to a micelle composition of DSPE-PEG2000:1V270 in a molar ratio of 90:10.

### Micelles

A micelle is an aggregate particle of amphiphilic molecules dispersed in a liquid colloid. Most micelles in aqueous solution form an aggregate particle with the hydrophilic head group in contact with the surrounding hydrophilic solvent, sequestering the hydrophobic tail regions in the micelle centre.

Among the micelle-forming compounds, micelles made of polyethylene glycol-phosphatidylethanolamine (PEG-PE) have particularly attractive properties such as good stability, longevity, and ability to accumulate in the areas with an abnormal vasculature via the enhanced permeability and retention effect (into the areas with leaky vasculature, such as tumors). Additionally, these micelles can be made “targeted” by attaching specific targeting ligand molecules to the micelle surface or can be comprised of stimuli-responsive amphiphilic block copolymers. Addition of second component such as surfactant or another hydrophobic material to the main micelle forming material further improves the solubilizing capacity of micelles without compromising their stability. Micelles are more simple to prepare compared to other nanoparticles e.g. liposomes, and may be produced by lipid mixture and sonication

The size of a micelle can be determined by various techniques known to a person of skill in the art. Dynamic light scattering (DLS) experiments may be performed using e.g. a Malvern Zetasizer Nano ZS instrument, suitable for measuring the size and size distribution of micelles formed in aqueous solution. The diameter measured in DLS is a value that refers to how a particle diffuses within a fluid and is referred to as a hydrodynamic diameter.

The diameter of the micelles disclosed herein is expressed as a numerical average.

In one embodiment, the diameter of the micelle disclosed herein is between 5 nm and 50 nm, such as between 6 and 46 nm, such as between 7 and 42 nm, such as between 8 and 38 nm, such as between 9 and 34 nm, such as between 10 and 34 nm, such as between 11 nm and 30 nm, such as between 12 nm and 26 nm.

In one embodiment, the diameter of the micelle is from 5 nm to 39 nm, such as from 5 nm to 20 nm, such as from 20 nm to 30 nm, such as from 30 nm to 35 nm, such as from 35 nm to 39 nm.

In one embodiment, the diameter of the micelle is from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm.

In one embodiment, the diameter of the micelle is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

An “amphiphilic micelle-forming agent” of the present disclosure may in some embodiments comprise a phospholipid. The structure of the phospholipid generally comprises two hydrophobic fatty acid “tails” and a hydrophilic “head” group comprising of a phosphate group. The two components are joined together by a glycerol molecule. The phosphate groups can be modified with simple organic molecules, such as choline, ethanolamine or serine. In some embodiments, the simple organic molecule acts as a linking group to a polymer, such as PEG.

In one embodiment, the amphiphilic micelle-forming agent is selected from the group consisting of: a poloxamer, a poloxamine, a PEG-polyester, a PEG-polyanhydride, a PEG-poly-amino acid, a phospholipid, a polysorbate, and a polyoxyethylene alkyl ether.

In one embodiment, the PEG-polyester is selected from the group consisting of: a PEG-poly(lactic acid) (PEG-PLA), a PEG-poly(lactic-co-glycolic acid) (PLGA), and a PEG-poly(ε-caprolactone) (PCL).

In one embodiment, the PEG-polyanhydride is a PEG-polysebacic anhydride (PSA).

In one embodiment, a micelle composition is provided comprising a PEG-poly-amino acid, wherein the PEG-poly-amino acid is selected from the group consisting of: a PEG-poly(L-histidine), a PEG-poly(L-aspartic acid), a PEG-poly(L-asparagine), a PEG-poly(L-glutamic acid), a PEG-poly(L-glutamine), and a PEG-poly(L-lysine).

In one embodiment, the amphiphilic micelle-forming agent is a phospholipid conjugated to polyethylene glycol (PEG). In one embodiment, the phospholipid conjugated to PEG is conjugated via a carbonyl group.

An example of a preferred amphiphilic micelle-forming agent is DSPE-PEG2000, exemplified below as an ammonium salt, i.e. DSPE-PEG2000 amine:

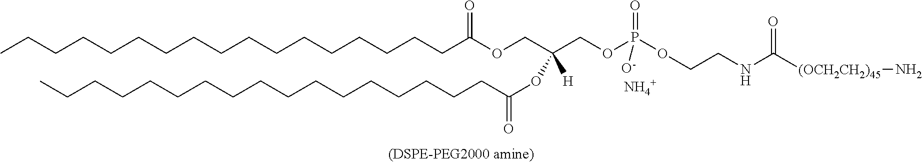

DSPE-PEG2000 comprises phosphatidylethanolamine, which is bound to PEG via a carbonyl group. Phosphatidylethanolamine comprises glycerol esterified with two fatty acids and phosphoric acid. Whereas the phosphate group is combined with choline in phosphatidylcholine, it is combined with the ethanolamine in phosphatidylethanolamine. The two fatty acids may be the same, or different, and are usually in the 1,2 positions, but can also be in the 1,3 positions. The terminal end of PEG is in some embodiments aminated i.e. bound to NH₂. The aminated variant of DSPE-PEG2000 is also referred to as DSPE-PEG2000 amine. In a most preferred embodiment, DSPE-PEG2000 is 1,2-distearyl-sn-glycero-3-phosphoethanolamine-N-[methoxy(polyethylene glycol)-2000].

### Polyethylene Glycol (PEG)

In one embodiment, the micelles of the present disclosure comprise PEG. In one embodiment, the PEG is in the form of PEG conjugated to a phospholipid. The size of the PEG is between PEG350 to PEG30.000.

In one embodiment, the size of PEG is between PEG350 and PEG5000, for example between PEG550 and PEG4000, for example between PEG750 and PEG3000, such as between PEG1000 and PEG3000, preferably the size of the PEG is PEG2000.

### Phospholipids of the Disclosure

The phospholipids disclosed herein may be part of the TLR7 agonist or the amphiphilic micelle-forming agent. Examples of phospholipids disclosed in the context of the amphiphilic micelle-forming agent may also be used as part of the TLR7 agonist, and vice versa.

In one embodiment, the micelle composition according to the present disclosure is provided, comprising a phospholipid, wherein the phospholipid comprises one or more alkyl chains that are C8-C24 alkyl(s), such as C10-C22, such as C12-C20, preferably C14-C18, most preferred C16-C18 saturated alkyl chains or unsaturated alkyl chains.

In one embodiment, the micelle composition according to the present disclosure is provided, comprising a phospholipid, wherein the phospholipid comprises a phosphatidylethanolamine (PE), a phosphatidylcholine (PC), a phosphatidylserine (PS), a phosphatidylglycerol (PG), a phosphatidylinositol (PI), a phosphatidic acid (PA), a bisphosphatidyl glycerol (DPG), or a phosphatidyl alcohol.

In one embodiment, the phosphatidylethanolamine is selected from the group consisting of 1,2-dioleoyl-phosphatidylethanolamine, 1,2-dipalmitoyl-phosphatidylethanolamine, 1,2-dimyristoyl-phosphatidylethanolamine, 1,2-distearoyl-phosphatidylethanolamine, 1-oleoyl-2-palmitoyl-phosphatidylethanolamine, 1-oleoyl-2-stearoyl-phosphatidylethanolamine, 1-palmitoyl-2-oleoyl-phosphatidylethanolamine, and 1-stearoyl-2-oleoyl-phosphatidylethanolamine.

In one embodiment, the phospholipid conjugated to PEG is selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one embodiment, a micelle composition is provided comprising a phospholipid conjugated to PEG, wherein the phospholipid is: 1,2-distearoyl-sn-glycero-3-phosphoethanolamine (DSPE)-PEG.

In one embodiment, the amphiphilic micelle-forming agent is DSPE-PEG2000.

### Other Amphiphilic Micelle Forming Agents of the Disclosure

Other amphiphilic micelle forming agents disclosed are amphiphilic organic compounds comprising a hydrophobic non-polar hydrocarbon moiety (tail) and a hydrophilic polar headgroup. The molecular structure of non-phospholipid amphiphiles have properties similar to amphiphilic phospholipids. Amphiphilic organic compounds of interest in the context of the present disclosure have a critical micelle concentration of 1 mM or less.

The amphiphilic micelle forming agents of the present disclosure have a hydrophobic tail and a hydrophilic headgroup providing a critical micelle concentration of 1 mM or less. In one embodiment, the amphiphilic micelle forming agent contains one or more aliphatic chains with at least 11 carbon atoms and a headgroup, wherein the amphiphilic micelle forming agent is preferably selected from a molecule comprising or consisting of one or more C6-C20 long carbon chains conjugated to a linker that is further conjugated to a hydrophilic headgroup. In one embodiment, the linker is glycerol, an amino acid linker, a phosphatidylethanolamine linker, or one or more carbohydrates. In one embodiment, the hydrophilic headgroup is PEG, one or more carbohydrates, one or more hydrophilic polymers, or any combination thereof. In some embodiments of the present disclosure, the amphiphilic micelle forming agent does not comprise a linker. In such cases, the hydrophobic tail and the hydrophilic headgroup are covalently connected by a single bond of any bond order.

Further amphiphilic organic compounds of the present disclosure include:
Poloxamers such as poloxamer 407, poloxamer 124, poloxamer f127, and poloxamer 188.
Tritons, such as Triton X-100, Triton X-114, Nonidet P-40 (NP-40), Igepal® CA-630
Glycosidic surfactants such as n-dodecyl-β-D-maltoside (DDM), beta-decyl-maltoside, octyl glucoside.
Tweens such as Tween-20, Tween-40, Tween 60 and Tween-80.

### Nanodiscs

Nanodiscs are another important nanosized structures of the present disclosure. The term nanodisc describes a small (5-50 nm in diameter) disc-shaped nanostructure that comprises two main components: Phospholipids, either synthetic or naturally occurring and a stabilizing belt that holds the phospholipids together which can be either a protein, a peptide or polymer. These stabilizing proteins are generally referred to as membrane scaffolding proteins (MSP) and align in double belt formation around the disc. Nanodiscs can be structurally very similar to discoidal high-density lipoproteins (HDL) and the MSPs are modified versions of apolipoprotein A1 (apoA1). Many of the apoA1 formed nanodiscs are approximately 10 nm and can be tuned in size by truncating the apoA1 protein.

Another way to form the nanodiscs is to use peptides where the lipid bilayer is surrounded by amphipathic peptides instead of two MSPs. Peptide nanodiscs are structurally similar to MSP nanodiscs and the peptides also align in a double belt. For the nanodiscs stabilized by polymers the phospholipids in the nanodisc are surrounded by polymers such as Styrene-maleic acid co-polymers (SMAs) or Diisobutylene-maleic acid (DIBMA).

In some embodiments, the nanodiscs of the present disclosure contain phospholipids selected from a group consisting of phosphatidylcholine (PC), phosphatidylethanolamine (PE), phosphatidylserine (PS), phosphatidylglycerol (PG), phosphatidylinositol (PI), phosphatidic acid (PA), DPG (bisphosphatidyl glycerol), and PEOH (phosphatidyl alcohol). In one embodiment, the nanodisc further comprises cholesterol, ergosterol and/or lanosterol.

In one embodiment of the present disclosure, a nanodisc is provided comprising a phosphatidylcholine selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine.

In a preferred embodiment of the present disclosure, the phosphatidylcholine is 1-palmitoyl-2-oleoyl-phosphatidylcholine (POPC).

In one embodiment, the nanodisc comprises a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one embodiment, the nanodisc is a lipoprotein which can either be a High Density Lipoprotein (HDL), Low Density Lipoprotein (LDL) or Very Low Density Lipoprotein (VLDL). Lipoproteins may be either naturally occurring particles or synthetic produced particles. Naturally occurring particles may be derived from blood or plasma from patients or healthy subjects or from cell culture systems. Lipoprotein particles can be fused with 1V270 in organic solvent or as a particle and injected into patients.

In one preferred embodiment of the present invention the formulation is a nanodisc with 1-25% molar concentration relative to the phospholipid concentration with a lipid conjugated TLR7 agonist embedded in the phospholipid containing part of the nanodisc.

In one embodiment, the nanodisc composition as disclosed herein is provided, wherein the composition comprises between 1% and 25% molar concentration of TLR7 agonist, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%. In a preferred embodiment the TLR7 agonist is 1V270.

### Liposomes

An embodiment of the present disclosure is a liposome, which, when relevant, contains alkyl chains of the phospholipids that are C8-C24, preferably C10-C22, more preferred C12-C20, preferably C14-C18, most preferred C16-C18 saturated chains or unsaturated chains and amounts of TLR7 agonist between 1-99%.

In one embodiment, a liposome is provided comprising the TLR7 agonist of the present disclosure, and a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine. In one embodiment, the liposome comprising the TLR7 agonist of the present disclosure is provided wherein the phospholipid comprises alkyl chains that are C8-C24, preferably C10-C22, more preferred C12-C20, preferably C14-C18, most preferred C16-C18 saturated chains or unsaturated chains and wherein the amount of the TLR7 agonist of the present disclosure is between 1-99%.

In one embodiment, a liposome is provided comprising the TLR7 agonist of the present disclosure, and a phospholipid conjugated to PEG. In one embodiment, a liposome is provided comprising the TLR7 agonist of the present disclosure, and a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one preferred embodiment of the present disclosure the liposome has 1-25% molar concentration of the TLR7 agonist relative to the phospholipid concentration, wherein the TLR7 agonist is embedded in the phospholipid containing part of the liposome.

In one embodiment, the liposome is provided, wherein the liposome comprises between 1% and 25% molar concentration of the TLR7 agonist of the present disclosure, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### Lipoplex

An embodiment of the present disclosure is a Lipoplex particle. In some embodiments, the lipoplex comprises a lipid selected from the group consisting of: DOPE-PEG2000, a phospholipid, cholesterol, 1V270, a charged lipid, and a nucleic acid sequence, such as DNA or RNA or mRNA or a modified nucleic acid sequence, and any combination thereof.

### Lipidoid

An embodiment of the present disclosure is a Lipidoid particle. In some embodiments, the lipidoid comprises a lipid selected from the group consisting of: DOPE-PEG2000, a phospholipid, cholesterol, a TLR7 agonist of the present disclosure, such as 1V270, a charged lipid, and a nucleic acid sequence, such as DNA or RNA or mRNA or a modified nucleic acid sequence, and any combination thereof.

### Emulsions

An embodiment of the present disclosure is an emulsion containing a lipid component and an aqueous component in which the lipid component partly contains amounts of the TLR7 agonist of the present disclosure between 1-99%. An emulsion of the TLR7 agonist of the present disclosure may be used for intramuscular or subcutaneous administration. In one embodiment, the emulsion of the present disclosure comprising the TLR7 agonist also contains one or more emulsifiers.

### Pharmaceutical Compositions

The micelles of the present disclosure are useful as constituents of a pharmaceutical formulation. Thus, in one embodiment, the micelle compositions of the present disclosure are pharmaceutical compositions. In one embodiment, a pharmaceutical composition comprising the micelle composition as defined herein is provided.

Any form of such formulation which is suitable for administration to a mammal is contemplated.

The pharmaceutical formulation according to the present disclosure is preferably in the form of a solution, dispersion, suspension, lyophilisate, or frozen form.

In one embodiment, the administration route may be intravenous, intratumoral, oral, subcutaneous, intradermal, intramuscular, nasal, intraperitoneal, pulmonary or renal administration.

In one embodiment, the micelle composition according to the present disclosure comprises the amphiphilic micelle-forming agent and the TLR7 agonist, wherein the molar ratio between the amphiphilic micelle-forming agent and the TLR7 agonist is from 50:50 to 99.5:0.5, such as from 60:40 to 99:1, such as from 70:30 to 98:2, such as from 80:20 to 95:5, for example 95:5, 90:10, or 80:20.

In one embodiment, the micelle composition as disclosed herein is provided, wherein the composition comprises between 1% and 25% molar concentration of TLR7 agonist, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

A micelle composition or pharmaceutical composition as defined herein is further provided, for use in the prevention, treatment or amelioration of a disease or disorder.

### Therapeutic Uses and Methods

The micelle composition of the present disclosure may be used in prophylaxis, treatment or amelioration of cancer, an infectious disease, an inflammatory condition or disease, an autoimmune disease or allergy. In one embodiment, the micelle composition of the present disclosure is used in treatment of cancer.

The micelle composition or pharmaceutical composition may thus be used for treatment of a cancer; an infectious disease; an inflammatory condition or disease; an autoimmune disease; or an allergy.

In one embodiment, the disease or disorder is cancer, such as colon cancer. In one embodiment, the disease or disorder is cancer, such as a solid tumor.

### Infectious Diseases

Another aspect of the disclosure is to provide a prevention or treatment for infectious diseases, by administration of the micelle composition disclosed herein to a subject. In a preferred embodiment, the micelle composition used to prevent or treat the infectious diseases is MBS8. Prevention or treatment of infectious diseases in both humans and livestock may be facilitated by the micelle composition disclosed herein. In one embodiment, the infectious disease is a viral infection or a bacterial infection. In a preferred embodiment, the treatment of infectious diseases is preventive. Thus in one embodiment, a method of preventing infectious disease is provided by administration of the micelle composition, such as MBS8, to a subject in need thereof. Suitable subjects for preventive treatment may be, without limitation, healthcare professionals and/or other humans working in close contact with infected subjects. These suitable subjects are at increased risk of getting infected, thus, preventive treatment by the micelle composition disclosed herein is advantageous.

### Combination Therapy

In one embodiment, the treatment of cancer is enhanced by combination of existing treatments like monoclonal antibodies (Trastuzumab, Rituximab, Cetuximab), radiotherapy, chemotherapy or immune checkpoint inhibitors like Pembrolizumab, Ipilimumab. Hence, in one embodiment, the treatment of cancer is a combination treatment further comprising administering a monoclonal antibody to the subject suffering from cancer.

As demonstrated in examples 11, 13, 14, 15, 16, 18 and 24 an enhancing or synergistic effect may be obtained when the micelle composition or pharmaceutical composition as disclosed herein is administered in combination with radiotherapy, chemotherapeutic agents or immune checkpoint inhibitors. Thus, in one embodiment, the treatment of cancer is a combination treatment further comprising radiotherapy. Certain types of chemotherapy are especially relevant for combination with TLR7 assets; these are chemotherapy compounds that induce what is called “immunogenic cell death” (ICD). As shown in example 14 and 24, micelle compositions comprising 1V270 significantly potentiate efficacy of doxorubicin and doxil and leads to effective treatment. In one embodiment, the treatment of cancer is a combination treatment further comprising administering a chemotherapeutic agent, such as doxorubicin or doxil. In one embodiment, the chemotherapeutic agent is selected from the group consisting of Doxorubicin, Doxil, Epirubicin, Cyclophosphamide, Bortezomib, and Oxaliplatin. In one embodiment, the treatment of cancer is a combination treatment further comprising administering immune checkpoint inhibitors, such as monoclonal antibodies targeting PD-1, PD-L1 or CTLA-4, such as α-PD-1, α-PD-L1 or α-CTLA-4, for example Atezolizumab, Avelumab, Durvalumab, Nivolumab, Tislelizumab, Pembrolizumab, or Ipilimumab. Preferably the immune checkpoint inhibitor is an α-PD-1, such as Nivolumab or Pembrolizumab. In a preferred embodiment, the micelle composition is MBS8 and the immune checkpoint inhibitor is Nivolumab or Pembrolizumab.

As demonstrated in example 18, the micelle composition is extremely effective in treating various cancers. This effect is demonstrated both as monotherapy and in combination with α-PD-1, even in treatment of cancers that do not respond to α-PD-1 monotherapy. In one embodiment, the combination treatment comprises administering the micelle composition and α-PD-1 for treatment of a cancer, in particular a cancer selected from the group consisting of: hepatoma, pancreatic, lymphoma, breast and colon cancer. In one embodiment, the combination treatment comprises administering the micelle composition and α-PD-1 for treatment of a cancer selected from the group consisting of: prostate cancer and kidney cancer. In particular the combination of the micelle composition and α-PD-1 is effective in treating cancers that do not respond to α-PD-1 monotherapy.

In one embodiment, the micelle composition used in combination treatment is MBS8. A preferred combination treatment is MBS8 and α-PD-1. In particular, this preferred combination treatment is effective in treating cancers that do not respond to anti-PD-1 monotherapy.

As shown in example 13, micelle compositions comprising 1V270 are very potent in combination with α-PD-1 leading to complete remission of at least 90% of treated mice carrying the CT26 tumor model. In one embodiment, MBS8 is administered in combination with a therapeutic antibody targeting the PD-1/PD-L1 pathway to a cancer patient in need of treatment. In one embodiment, the therapeutic antibody targeting the PD-1/PD-L1 pathway is selected from the group consisting of: Atezolizumab, Avelumab, Durvalumab, Nivolumab, Pembrolizumab, Spartalizumab/PDR001, Tislelizumab, BCD-100, TSR-042, Camrelizumab, IB1308, KN035, and CS1001.

Monoclonal antibodies are beneficial in combination with TLR7 agonists through the activation of the complement-dependent cytotoxicity (CDC), antibody-dependent cellular cytotoxicity (ADCC), and antibody-dependent cellular phagocytosis (ADCP). This is relevant for antibodies against e.g. CD20, EGFR, CD38 and HER2. The ADCC, ADCP and CDC mediated tumor cell killing is dependent on activated NK-cells, macrophages and neutrophils which are activated by the micelle composition of the present disclosure (FIG. 16), in particular MBS8.

In one embodiment, MBS8 is administered to a cancer patient in combination with a monoclonal antibody targeting CD20.

In one embodiment, MBS8 is administered to a cancer patient in combination with a monoclonal antibody targeting the epidermal growth factor receptor (EGFR).

In one embodiment, MBS8 is administered to a cancer patient in combination with a monoclonal antibody targeting the Human Epidermal Growth Factor Receptor 2 (HER2).

In one embodiment, MBS8 is administered to a cancer patient in combination with a monoclonal antibody targeting CD38. In a particular embodiment, the monoclonal antibody targeting CD38 is selected from Daratumumab and Isatuximab.

In one embodiment, MBS8 is administered to a cancer patient in combination with a monoclonal antibody selected from the group consisting of: Ublituximab, Obinutuzumab, Ofatumumab, Ibritumomab tiuxetan, Rituximab, Tositumomab, Depatuxizumab mafodotin, Necitumumab, Panitumumab, Cetuximab, Trastuzumab, Trastuzumab-dkst, Trastuzumab emtansine, BAT8001, Pertuzumab, Margetuximab, Trastuzumab deruxtecan, Trastuzumab duocarmazine, Daratumumab, and Isatuximab.

In one embodiment, MBS8 is administered in combination with an antibody targeting CD47, such as Magrolimab.

In one embodiment, the treatment of cancer is performed as monotherapy, comprising administration of the micelle composition or pharmaceutical composition as disclosed herein.

The term “prophylaxis”, as used herein, refers to prevention of a disease or prevention of spreading of a disease.

The term “treatment”, as used herein, refers to the combating of a disease or disorder. “Treatment” or “treating,” as used herein, includes any desirable effect on the symptoms or pathology of a disease or condition as described herein, and may include even minimal changes or improvements in one or more measurable markers of the disease or condition being treated. “Treatment” or “treating” does not necessarily indicate complete eradication or cure of the disease or condition, or associated symptoms thereof.

The term “amelioration”, as used herein, refers to moderation in the severity of the symptoms of a disease or condition. Improvement in a patient's condition, or the activity of making an effort to correct, or at least make more acceptable, conditions that are difficult to endure related to patient's conditions is considered “ameliorative” treatment.

In one embodiment, the micelle composition is used in prophylaxis, treatment or amelioration of cancer.

In one embodiment, the micelle composition is used in prophylaxis, treatment or amelioration of an infectious disease.

In one embodiment, a method for in vivo activation of immune cells in a subject is provided, comprising administering the micelle composition or pharmaceutical composition as defined herein to said subject in an amount sufficient to activate said immune cells. A preferred subject is a human being, such as human being suffering from cancer.

### TLR7 Agonists

Toll-like receptor 7, also known as TLR7, is a protein that in humans is encoded by the TLR7 gene. It is a member of the toll-like receptor (TLR) family and plays an important role in pathogen recognition and activation of innate immunity. Due to their ability to induce robust production of anti-cancer cytokines such as interleukin-12, TLR7 agonists have been investigated for cancer immunotherapy.

The micelle compositions of the present disclosure comprises a toll-like receptor 7 (TLR7) agonist of formula (I), formula (II), formula (III) or formula (IV);

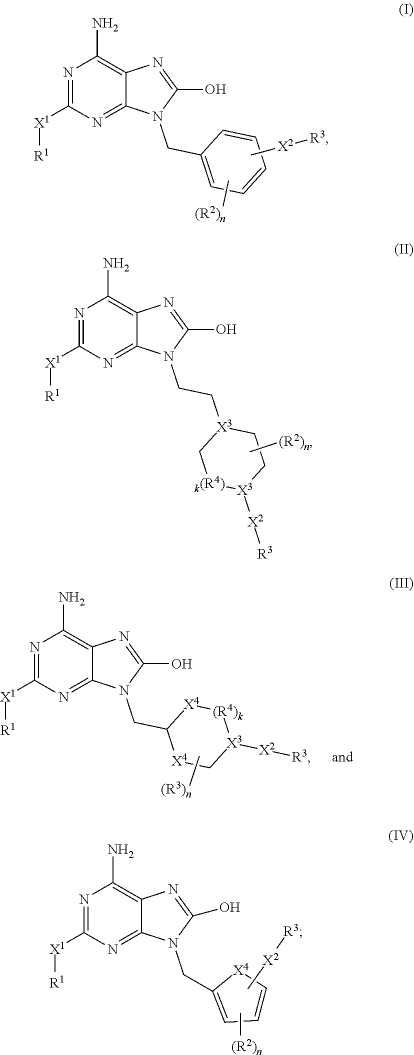

wherein X¹ is —O—, —S— or —NR^{C};
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R^{C} is hydrogen, C₁₋₁₀alkyl, or substituted C₁₋₁₀alkyl; or R^{C} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxy C₁₋₆alkylene, amino, cyano, halo, or aryl;
n is 0, 1, 2, 3 or 4;
X² is a bond or a linking group; and
R³ is a lipid;

### X³ is —N— or —CH—;

R⁴ is —CH₂— or —CH(R²)—; and
k is 0 or 1;
X⁴ is —O—, —S—, —NH—, —N(R^{d})—, —CH₂—, or —CH(R²)—;
each R^{d} is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)O(C₁-C₆)alkyl (alkoxycarbonyl), substituted —C(O)O(C₁-C₆)alkyl, —C(O)NR^{a}R^{b} (carbamoyl);
or a tautomer thereof;
or a pharmaceutically acceptable salt or solvate thereof, and
wherein the ring system of formula (II) is a piperidine ring with one heteroatom being an N atom and with the N-atom of the piperidine ring adjacent to X², and
wherein the purine group in any of Formula (I), (II), (III), or (IV) is subject to tautomeric rearrangements;
and an amphiphilic micelle-forming agent.

It is to be understood that the purine group in any of Formula (I), (II), (III), or (IV) is subject to tautomeric rearrangements.

In one embodiment, the micelle composition according to the present disclosure is provided, wherein the TLR7 agonist is of formula (I):

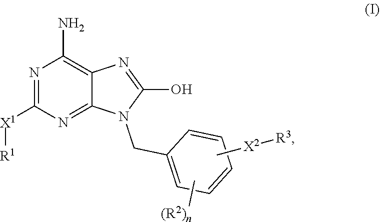

wherein X¹ is —O—, —S—, or —NR^{C};
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R^{C} is hydrogen, C₁₋₁₀alkyl, or substituted C₁₋₁₀alkyl; or R^{C} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxy C₁₋₆alkylene, amino, cyano, halo, or aryl;
n is 0, 1, or 2;
X² is a bond or a linking group; and
R³ is a lipid;
or a tautomer thereof;
or a pharmaceutically acceptable salt or solvate thereof, and
wherein the purine group is subject to tautomeric rearrangements.

In one embodiment, the micelle composition according to the present disclosure is provided, wherein the TLR7 agonist is of formula (I):

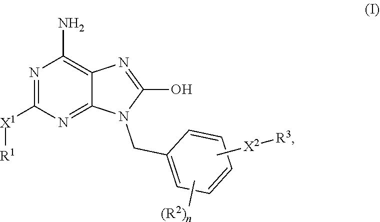

wherein X¹ is —O—, —S—, or —NR^{C};
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R^{C} is hydrogen, C₁₋₁₀alkyl, or substituted C₁₋₁₀alkyl; or R^{C} and R¹ taken together with the nitrogen to which they are attached form a heterocyclic ring or a substituted heterocyclic ring;
R² is absent;
n is 0;
X² is a bond or a linking group; and
R³ is a lipid;
or a tautomer thereof;
or a pharmaceutically acceptable salt or solvate thereof, and
wherein the purine group is subject to tautomeric rearrangements. It is understood by a person of skill in the art that when R² is absent, n must be 0 i.e. not present.

In one embodiment, the micelle composition according to the present disclosure is provided, wherein the TLR7 agonist is of formula (I):

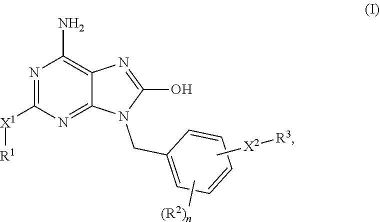

wherein X¹ is —O—
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R² is absent;
n is 0;
X² is a bond or a linking group; and
R³ is a lipid;
or a tautomer thereof;
or a pharmaceutically acceptable salt or solvate thereof, and
wherein the purine group is subject to tautomeric rearrangements.

In one embodiment, the micelle composition according to the present disclosure is provided, wherein the TLR7 agonist is of formula (V):

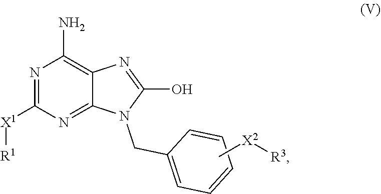

wherein X¹ is —O—
R¹ is hydrogen, (C₁-C₆)alkyl, substituted (C₁-C₆)alkyl, C₆aryl, or substituted C₆aryl, C₅₋₆heterocyclic, substituted C₅₋₆heterocyclic;
X² is a bond or a linking group; and
R³ is a lipid;
or a tautomer thereof;
or a pharmaceutically acceptable salt or solvate thereof, and
wherein the purine group is subject to tautomeric rearrangements.

In one embodiment, the micelle composition according to the present disclosure is provided, wherein the TLR7 agonist is of formula (VI):

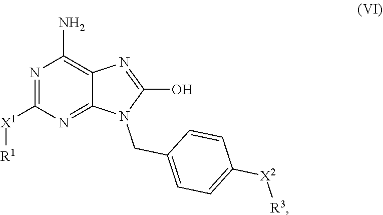

wherein X¹ is —O—
R¹ is hydrogen, (C₁-C₆)alkyl, substituted (C₁-C₆)alkyl, C₆aryl, or substituted C₆aryl, C₅₋₆ heterocyclic, substituted C₅₋₆heterocyclic;
X² is a bond or a linking group; and
R³ is a lipid;
or a tautomer thereof;
or a pharmaceutically acceptable salt or solvate thereof, and
wherein the purine group is subject to tautomeric rearrangements.

In one embodiment, the micelle composition according to the present disclosure is provided, wherein the TLR7 agonist is of formula (VII):

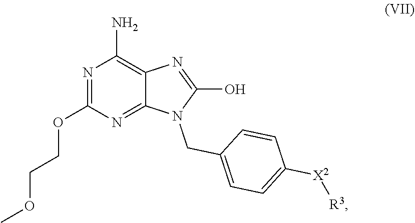

wherein X² is a bond or a linking group; and
R³ is a lipid;
or a tautomer thereof;
or a pharmaceutically acceptable salt or solvate thereof, and
wherein the purine group is subject to tautomeric rearrangements.

In one embodiment, the micelle composition according to the present disclosure is provided, wherein the TLR7 agonist is of formula (VIII):

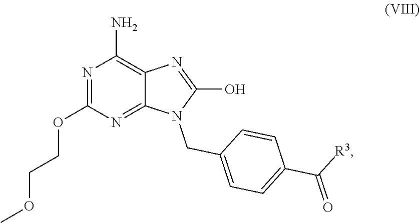

wherein R³ is a lipid;
or a tautomer thereof;
or a pharmaceutically acceptable salt or solvate thereof, and
wherein the purine group is subject to tautomeric rearrangements.

In one embodiment, X² is selected from the group consisting of: a bond, —O—, —C(O)-(carbonyl), (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₅-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl); and

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
and wherein
X¹ is —O—, —S—, or —NR^{C};
R¹ is hydrogen, (C₁-C₆)alkyl, or substituted (C₁-C₆)alkyl;
R^{C} is hydrogen, C₁₋₆alkyl, or substituted C₁₋₆alkyl; or R^{C} and R¹ taken together with the nitrogen to which they are attached form a heterocyclic ring or a substituted heterocyclic ring.

In a preferred embodiment, X² is —C(O)— (carbonyl). In a preferred embodiment, X² is —C(O)— (carbonyl) and R³ is 1,2-dioleoyl-phosphatidylethanolamine according to the formulas disclosed herein.

In one embodiment, R³ is a lipid selected from the group consisting of: a phospholipid comprising one or two carboxylic esters, a gonane, such as cholesterol, a saccharolipid, and a glyceride. In one embodiment, R³ is a phospholipid comprising one or two carboxylic esters.

A “lipid” as disclosed herein refers to a group of substances comprising at least one hydrophobic part, which by itself would be insoluble in water. Exemplary groups of lipids may without limitation be fatty acids, glycerolipids, glycerophospholipids, sphingolipids, sterol lipids, prenol lipids, saccharolipids, and polyketides.

In a particularly preferred embodiment, the TLR7 agonist according to Formula (I) has a structure according to Formula (IA), wherein the definitions of Formula (I) are as follows:
X¹ is —O—;
R¹ is 2-methoxy-1-ethyl
R² is absent;
X² is carbonyl; and
R³ is 1,2-dioleoyl-sn-glycero-3-phosphoethanolamine (DOPE);
or a pharmaceutically acceptable salt or solvate thereof;

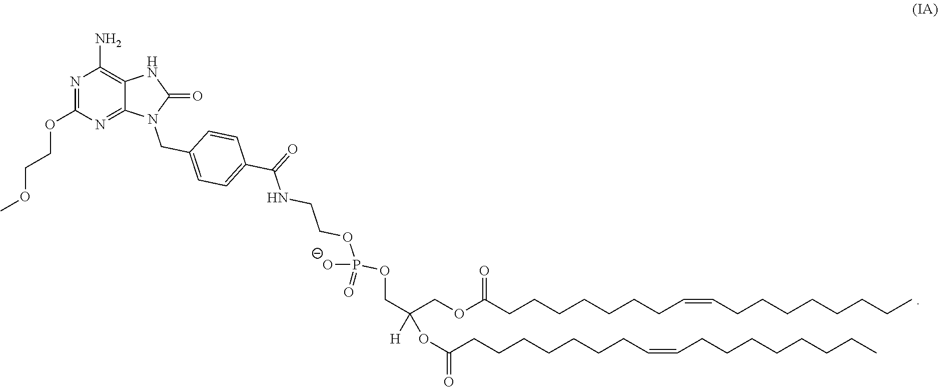 [SMILES: CCCCCCCC/C=C\CCCCCCCC(=O)OCC(COP(=O)([O-])OCCNC(=O)c1ccc(Cn2c(=O)[nH]c3c(N)nc(OCCOC)nc32)cc1)OC(=O)CCCCCCC/C=C\CCCCCCCC]

The compound according to Formula (IA) is also known in the literature as 1V270 from e.g. U.S. Pat. No. 8,357,374.

Thus, in a preferred embodiment, the micelle composition is provided, wherein the TLR7 agonist has a structure according to formula (IA):

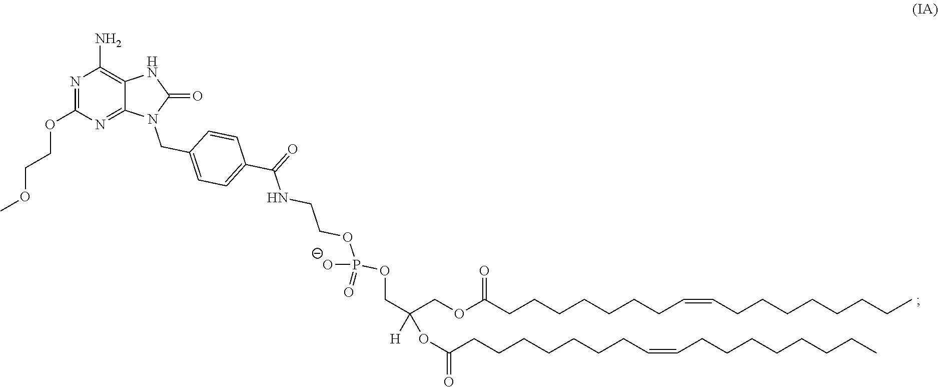 [SMILES: CCCCCCCC/C=C\CCCCCCCC(=O)OCC(COP(=O)([O-])OCCNC(=O)c1ccc(Cn2c(=O)[nH]c3c(N)nc(OCCOC)nc32)cc1)OC(=O)CCCCCCC/C=C\CCCCCCCC]

or a tautomer thereof;

A halogen atom as referred to herein refers to a fluorine atom, chlorine atom, bromine atom or iodine atom.

Aryl refers to a C6-10 monocyclic or fused cyclic aryl group, such as phenyl, indenyl, or naphthyl, and the like.

Heterocyclic or heterocycle (Het) refers to monocyclic saturated heterocyclic groups, or unsaturated monocyclic or fused heterocyclic group containing at least one heteroatom, e.g., 0-3 nitrogen atoms, 0-1 oxygen atom (—O—), and 0-1 sulfur atom (—S—). Non-limiting examples of saturated monocyclic heterocyclic group includes 5 or 6 membered saturated heterocyclic group, such as tetrahydrofuranyl, pyrrolidinyl, morpholinyl, piperidyl, piperazinyl or pyrazolidinyl. Non-limiting examples of unsaturated monocyclic heterocyclic group includes 5 or 6 membered unsaturated heterocyclic group, such as furyl, pyrrolyl, pyrazolyl, imidazolyl, thiazolyl, thienyl, pyridyl or pyrimidinyl. Non-limiting examples of unsaturated fused heterocyclic groups includes unsaturated bicyclic heterocyclic group, such as indolyl, isoindolyl, quinolyl, benzothizolyl, chromanyl, benzofliranyl, and the like. A Het group can be a saturated heterocyclic group or an unsaturated heterocyclic group, such as a heteroaryl group.

Non-limiting examples of heterocyclic rings include 5 or 6 membered saturated heterocyclic rings, such as 1-pyrrolidinyl, 4-morpholinyl, 1-piperidyl, 1-piperazinyl or 1-pyrazolidinyl, 5 or 6 membered unsaturated heterocyclic rings such as 1-imidazolyl, and the like.

The alkyl, aryl, heterocyclic groups of R¹ can be optionally substituted with one or more substituents, wherein the substituents are the same or different, and include lower alkyl; cycloalkyl, hydroxyl; hydroxy C1-6 alkylene, such as hydroxymethyl, 2-hydroxyethyl or 3-hydroxypropyl; lower alkoxy; C1-6 alkoxy C1-6 alkyl, such as 2-methoxyethyl, 2-ethoxyethyl or 3-methoxypropyl; amino; alkylamino; dialkyl amino; cyano; nitro; acyl; carboxyl; lower alkoxycarbonyl; halogen; mercapto; C1-6 alkylthio, such as, methylthio, ethylthio, propylthio or butylthio; substituted C1-6 alkylthio, such as methoxyethylthio, methylthioethylthio, hydroxyethylthio or chloroethylthio; aryl; substituted C6-10 monocyclic or fused-cyclic aryl, such as 4-hydroxyphenyl, 4-methoxyphenyl, 4-fluorophenyl, 4-chlorophenyl or 3,4-dichlorophenyl; 5-6 membered unsaturated heterocyclic, such as furyl, pyrrolyl, pyrazolyl, imidazolyl, thiazolyl, thienyl, pyridyl or pyrimidinyl; and bicyclic unsaturated heterocyclic, such as indolyl, isoindolyl, quinolyl, benzothiazolyl, chromanyl, benzofuranyl or phthalimino. In certain embodiments, one or more of the above groups can be expressly excluded as a substituent of various other groups of the formulas. In some embodiments, the five-membered ring of the formula is a thiazole ring.

The alkyl, aryl, heterocyclic groups of R² can be optionally substituted with one or more substituents, wherein the substituents are the same or different, and include hydroxyl; C1-6 alkoxy, such as methoxy, ethoxy or propoxy; carboxyl; C2-7 alkoxycarbonyl, such as methoxycarbonyl, ethoxycarbonyl or propoxycarbonyl) and halogen. The alkyl, aryl, heterocyclic groups of R^{c} can be optionally substituted with one or more substituents, wherein the substituents are the same or different, and include C3-6 cycloalkyl; hydroxyl; C1-6 alkoxy; amino; cyano; aryl; substituted aryl, such as 4-hydroxyphenyl, 4-methoxyphenyl, 4-chlorophenyl or 3,4-dichlorophenyl; nitro and halogen.

The heterocyclic ring formed together with R^{c} and R¹ and the nitrogen atom to which they are attached can be optionally substituted with one or more substituents, wherein the substituents are the same or different, and include C₁₋₆ alkyl; hydroxy C₁₋₆ alkylene; C₁₋₆ alkoxy C₁₋₆ alkylene; hydroxyl; C₁₋₆ alkoxy; and cyano.

In one embodiment, the micelle composition as defined herein is provided, wherein: the TLR7 agonist is of formula (IA);

the amphiphilic micelle-forming agent is DSPE-PEG2000; and
the ratio between the TLR7 agonist and amphiphilic micelle-forming agent is 95:5, 90:10, or 80:20.

In a preferred embodiment, the micelle composition as defined herein is provided, wherein: the TLR7 agonist is of formula (IA);

the amphiphilic micelle-forming agent is DSPE-PEG2000; and
the molar ratio between the amphiphilic micelle-forming agent and the TLR7 agonist is 95:5, 90:10, or 80:20.

### TLR7-Agonist: TMX-201 (1V270)

In one embodiment, a composition is provided comprising the TLR7 agonist 1V270 and one or more amphiphilic micelle forming agents. In one embodiment, the TLR7 agonist is 1V270,

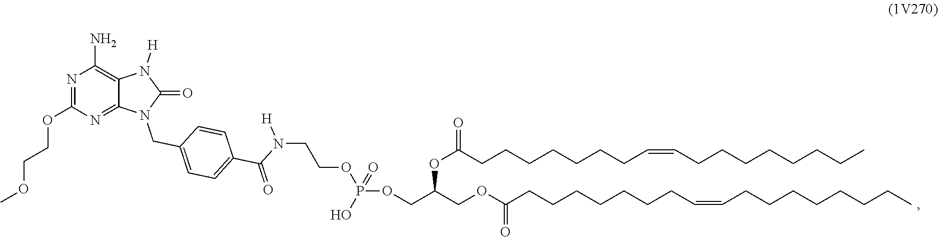 [SMILES: CCCCCCCC/C=C\CCCCCCCC(=O)OC[C@@H](COP(=O)(O)OCCNC(=O)c1ccc(Cn2c(=O)[nH]c3c(N)nc(OCCOC)nc32)cc1)OC(=O)CCCCCCC/C=C\CCCCCCCC]

or a pharmaceutically acceptable salt thereof.

The TLR7 agonist 1V270 can be co-formulated with one or more amphiphilic micelle forming agents. These amphiphilic micelle forming agents of the present disclosure have a hydrophobic tail and a hydrophilic headgroup providing a critical micelle concentration of 1 mM or less. In one embodiment, the amphiphilic micelle forming agent contains one or more aliphatic chains with at least 11 carbon atoms and a headgroup, wherein the amphiphilic micelle forming agent is preferably selected from a molecule comprising or consisting of one or more C₆-C₂₀ long carbon chains conjugated to a linker that is further conjugated to a hydrophilic headgroup. In one embodiment, the linker is glycerol or one or more carbohydrates. In one embodiment, the hydrophilic headgroup is PEG, one or more carbohydrates, one or more hydrophilic polymers, or any combination thereof.

In one embodiment, 1V270 is co-formulated with a further amphiphilic organic compound of the present disclosure, including:
Poloxamers such as poloxamer 407, poloxamer 124, poloxamer f127, and poloxamer 188.
Tritons, such as Triton X-100, Triton X-114, Nonidet P-40 (NP-40), Igepal® CA-630
Glycosidic surfactant such as n-dodecyl-β-D-maltoside (DDM), beta-decyl-maltoside, octyl glucoside.
Tweens such as Tween-20, Tween-40, Tween 60, and Tween-80.

### 1V270 Liposome

In one embodiment, a liposome is provided comprising 1V270, and a phospholipid.

In one embodiment, a liposome is provided comprising 1V270, and a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine. In one embodiment, the liposome comprising 1V270 is provided wherein the phospholipid comprises alkyl chains that are C8-C24, preferably C10-C22, more preferred C12-C20, preferably C14-C18, most preferred C16-C18 saturated chains or unsaturated chains and wherein the amount of 1V270 is between 1-99%.

In one embodiment, a liposome is provided comprising 1V270, and a phospholipid conjugated to PEG. In one embodiment, a liposome is provided comprising 1V270, and a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one preferred embodiment of the present disclosure the liposome has 1-25% molar concentration of 1V270 relative to the phospholipid concentration, wherein 1V270 is embedded in the phospholipid containing part of the liposome.

In one embodiment, the liposome is provided, wherein the liposome comprises between 1% and 25% molar concentration of 1V270, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### 1V270 Nanodisc

In one embodiment, a nanodisc is provided comprising 1V270, a phospholipid, and a membrane scaffolding protein (MSP).

In one embodiment, a nanodisc is provided comprising 1V270, a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine, and a membrane scaffolding protein (MSP). In one embodiment, the nanodisc comprising 1V270 has a diameter from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm. In one embodiment, the diameter of this nanodisc is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

In one embodiment, a nanodisc is provided comprising 1V270, a phospholipid conjugated to PEG, and a membrane scaffolding protein (MSP).

In some embodiments, the nanodiscs of the present disclosure comprise 1V270 and one or more phospholipids selected from a group consisting of phosphatidylcholine (PC), phosphatidylethanolamine (PE), phosphatidylserine (PS), phosphatidylglycerol (PG), phosphatidylinositol (PI), phosphatidic acid (PA), DPG (bisphosphatidyl glycerol), and PEOH (phosphatidyl alcohol). In one embodiment, the nanodisc further comprises cholesterol, ergosterol and/or lanosterol.

In one embodiment, a nanodisc is provided comprising 1V270, a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG, and a membrane scaffolding protein (MSP).

In one preferred embodiment of the present disclosure the nanodisc has 1-25% molar concentration of 1V270 relative to the phospholipid concentration, wherein 1V270 is embedded in the phospholipid containing part of the nanodisc.

In one embodiment, the nanodisc is provided, wherein the nanodisc comprises between 1% and 25% molar concentration of 1V270, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

In one embodiment, the nanodisc comprising 1V270 is a lipoprotein which can either be a High Density Lipoprotein (HDL), Low Density Lipoprotein (LDL) or Very Low Density Lipoprotein (VLDL). The lipoproteins comprising 1V270 may be either naturally occurring particles or synthetic produced particles. Naturally occurring particles may be derived from blood or plasma from patients or healthy subjects or from cell culture systems. Lipoprotein particles can be fused with 1V270 in organic solvent or as a particle and injected into patients.

### 1V270 Lipoplex

In one embodiment, a Lipoplex of 1V270 is provided. In one embodiment, the lipoplex of 1V270 comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipoplex of 1V270 also comprises cholesterol. In one embodiment, the Lipoplex of 1V270 also comprises a charged lipid, a nucleic acid sequence, such as DNA or RNA or mRNA or a modified nucleic acid sequence, or any combination thereof. In one embodiment, the Lipoplex is provided, wherein the composition comprises between 1% and 25% molar concentration of 1V270, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### 1V270 Lipidoid

In one embodiment, a Lipidoid of 1V270 is provided. In one embodiment, the Lipidoid of 1V270 comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipidoid of 1V270 also comprises cholesterol. In one embodiment, the Lipidoid of 1V270 also comprises a charged lipid, a nucleic acid sequence, such as DNA or RNA or mRNA or a modified nucleic acid sequence, or any combination thereof. In one embodiment, the Lipidoid is provided, wherein the composition comprises between 1% and 25% molar concentration of 1V270, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### Emulsions of 1V270

In one embodiment of the present disclosure, an emulsion is provided containing a lipid component and an aqueous component in which the lipid component partly contains amounts of 1V270 between 1-99%. An emulsion of 1V270 may preferably be used for intramuscular or subcutaneous administration. In one embodiment, the emulsion of the present disclosure comprising 1V270 also contains one or more emulsifiers.

### TLR7-Agonist: TMX-202

In one embodiment, a composition is provided comprising the TLR7 agonist TMX-202 and one or more amphiphilic micelle forming agents herein described. In one embodiment, the TLR7 agonist is TMX-202,

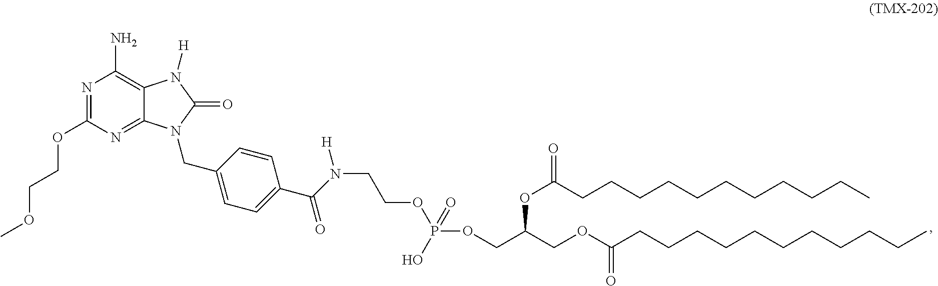 [SMILES: CCCCCCCCCCCC(=O)OC[C@@H](COP(=O)(O)OCCNC(=O)c1ccc(Cn2c(=O)[nH]c3c(N)nc(OCCOC)nc32)cc1)OC(=O)CCCCCCCCCCC]

or a pharmaceutically acceptable salt thereof.

The TLR7 agonist TMX-202 can be co-formulated with one or more amphiphilic micelle forming agents. These amphiphilic micelle forming agents of the present disclosure have a hydrophobic tail and a hydrophilic headgroup providing a critical micelle concentration of 1 mM or less. In one embodiment, the amphiphilic micelle forming agent contains one or more aliphatic chains with at least 11 carbon atoms and a headgroup, wherein the amphiphilic micelle forming agent is preferably selected from a molecule comprising or consisting of one or more C6-C20 long carbon chains conjugated to a linker that is further conjugated to a hydrophilic headgroup. In one embodiment, the linker is glycerol or one or more carbohydrates. In one embodiment, the hydrophilic headgroup is PEG, one or more carbohydrates, one or more hydrophilic polymers, or any combination thereof.

In one embodiment, TMX-202 is co-formulated with a further amphiphilic organic compound of the present disclosure, including:
Poloxamers such as poloxamer 407, poloxamer 124, poloxamer f127, and poloxamer 188.
Tritons, such as Triton X-100, Triton X-114, Nonidet P-40 (NP-40), Igepal® CA-630
Glycosidic surfactant such as n-dodecyl-β-D-maltoside (DDM), beta-decyl-maltoside, octyl glucoside.
Tweens such as Tween-20, Tween-40, Tween 60, and Tween-80.

### TMX-202 Micelle

In some embodiments, the micelle compositions of the present disclosure comprise the TLR7 agonist, TMX-202. In one embodiment, the diameter of the micelle disclosed herein with TMX-202 is between 5 nm and 50 nm, such as between 6 and 46 nm, such as between 7 and 42 nm, such as between 8 and 38 nm, such as between 9 and 34 nm, such as between 10 and 34 nm, such as between 11 nm and 30 nm, such as between 12 nm and 26 nm.

In one embodiment, the diameter of the micelle with TMX-202 is from 5 nm to 39 nm, such as from 5 nm to 20 nm, such as from 20 nm to 30 nm, such as from 30 nm to 35 nm, such as from 35 nm to 39 nm.

In one embodiment, the diameter of the micelle is from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm.

In one embodiment, the diameter of the micelle with TMX-202 is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

In one embodiment, the composition according to the present disclosure is provided which is a micelle composition, comprising an amphiphilic micelle-forming agent and TMX-202. In one embodiment, the amphiphilic micelle-forming agent comprises one or more alkyl chains that are C8-C24 alkyl(s), such as C10-C22, such as C12-C20, preferably C14-C18, most preferred C16-C18 saturated alkyl chains or unsaturated alkyl chains.

In one embodiment, the micelle composition comprising TMX-202 according to the present disclosure is provided, comprising a phospholipid, wherein the phospholipid comprises a phosphatidylethanolamine (PE), a phosphatidylcholine (PC), a phosphatidylserine (PS), a phosphatidylglycerol (PG), a phosphatidylinositol (PI), a phosphatidic acid (PA), a bisphosphatidyl glycerol (DPG), or a phosphatidyl alcohol.

In one embodiment, the phosphatidylethanolamine is selected from the group consisting of 1,2-dioleoyl-phosphatidylethanolamine, 1,2-dipalmitoyl-phosphatidylethanolamine, 1,2-dimyristoyl-phosphatidylethanolamine, 1,2-distearoyl-phosphatidylethanolamine, 1-oleoyl-2-palmitoyl-phosphatidylethanolamine, 1-oleoyl-2-stearoyl-phosphatidylethanolamine, 1-palmitoyl-2-oleoyl-phosphatidylethanolamine, and 1-stearoyl-2-oleoyl-phosphatidylethanolamine.

In one embodiment, the phospholipid conjugated to PEG is selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one embodiment, a micelle composition comprising TMX-202 is provided comprising a phospholipid conjugated to PEG, wherein the phospholipid is: 1,2-distearoyl-sn-glycero-3-phosphoethanolamine (DSPE)-PEG. In one embodiment, the amphiphilic micelle-forming agent is DSPE-PEG2000.

### TMX-202 Liposome

In one embodiment, a liposome is provided comprising TMX-202, and a phospholipid.

In one embodiment, a liposome is provided comprising TMX-202, and a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine. In one embodiment, the liposome comprising TMX-202 is provided wherein the phospholipid comprises alkyl chains that are C8-C24, preferably C10-C22, more preferred C12-C20, preferably C14-C18, most preferred C16-C18 saturated chains or unsaturated chains and wherein the amount of TMX-202 is between 1-99%.

In one embodiment, a liposome is provided comprising TMX-202, and a phospholipid conjugated to PEG. In one embodiment, a liposome is provided comprising TMX-202, and a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one preferred embodiment of the present disclosure the liposome has 1-25% molar concentration of TMX-202 relative to the phospholipid concentration, wherein TMX-202 is embedded in the phospholipid containing part of the liposome.

In one embodiment, the liposome is provided, wherein the liposome comprises between 1% and 25% molar concentration of TMX-202, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### TMX-202 Nanodisc

In one embodiment, a nanodisc is provided comprising TMX-202, a phospholipid, and a membrane scaffolding protein (MSP).

In one embodiment, a nanodisc is provided comprising TMX-202, a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine, and a membrane scaffolding protein (MSP). In one embodiment, the nanodisc comprising TMX-202 has a diameter from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm. In one embodiment, the diameter of this nanodisc is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

In one embodiment, a nanodisc is provided comprising TMX-202, a phospholipid conjugated to PEG, and a membrane scaffolding protein (MSP).

In some embodiments, the nanodiscs of the present disclosure comprise TMX-202 and one or more phospholipids selected from a group consisting of phosphatidylcholine (PC), phosphatidylethanolamine (PE), phosphatidylserine (PS), phosphatidylglycerol (PG), phosphatidylinositol (PI), phosphatidic acid (PA), DPG (bisphosphatidyl glycerol), and PEOH (phosphatidyl alcohol). In one embodiment, the nanodisc further comprises cholesterol, ergosterol and/or lanosterol.

In one embodiment, a nanodisc is provided comprising TMX-202, a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG, and a membrane scaffolding protein (MSP).

In one preferred embodiment of the present disclosure the nanodisc has 1-25% molar concentration of TMX-202 relative to the phospholipid concentration, wherein TMX-202 is embedded in the phospholipid containing part of the nanodisc.

In one embodiment, the nanodisc is provided, wherein the nanodisc comprises between 1% and 25% molar concentration of TMX-202, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

In one embodiment, the nanodisc comprising TMX-202 is a lipoprotein which can either be a High Density Lipoprotein (HDL), Low Density Lipoprotein (LDL) or Very Low Density Lipoprotein (VLDL). The lipoproteins comprising TMX-202 may be either naturally occurring particles or synthetic produced particles. Naturally occurring particles may be derived from blood or plasma from patients or healthy subjects or from cell culture systems. Lipoprotein particles can be fused with TMX-202 in organic solvent or as a particle and injected into patients.

### TMX-202 Lipoplex

In one embodiment, a Lipoplex of TMX-202 is provided. In one embodiment, the lipoplex of TMX-202 comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipoplex of TMX-202 also comprises cholesterol. In one embodiment, the Lipoplex of TMX-202 also comprises a charged lipid, a nucleic acid sequence, such as DNA or RNA or mRNA or a modified nucleic acid sequence, or any combination thereof. In one embodiment, the Lipoplex is provided, wherein the composition comprises between 1% and 25% molar concentration of TMX-202, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### TMX-202 Lipidoid

In one embodiment, a Lipidoid of TMX-202 is provided. In one embodiment, the Lipidoid of TMX-202 comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipidoid of TMX-202 also comprises cholesterol. In one embodiment, the Lipidoid of TMX-202 also comprises a charged lipid, a nucleic acid sequence, such as DNA or RNA or mRNA or a modified nucleic acid sequence, or any combination thereof. In one embodiment, the Lipidoid is provided, wherein the composition comprises between 1% and 25% molar concentration of TMX-202, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### Emulsions of TMX-202

In one embodiment of the present disclosure, an emulsion is provided containing a lipid component and an aqueous component in which the lipid component partly contains amounts of TMX-202 between 1-99%. An emulsion of TMX-202 may preferably be used for intramuscular or subcutaneous administration. In one embodiment, the emulsion of the present disclosure comprising TMX-202 also contains one or more emulsifiers.

### TLR7-Agonist: CMPD 5

In one embodiment, a composition is provided comprising the TLR7 agonist CMPD 5 and one or more amphiphilic micelle forming agents. In one embodiment, the TLR7 agonist is CMPD 5,

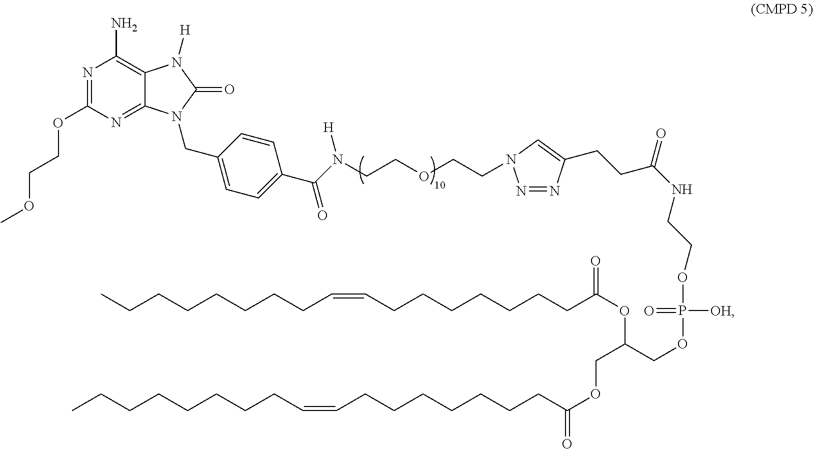

or a pharmaceutically acceptable salt thereof.

The TLR7 agonist CMPD 5 can be co-formulated with one or more amphiphilic micelle forming agents. These amphiphilic micelle forming agents of the present disclosure have a hydrophobic tail and a hydrophilic headgroup providing a critical micelle concentration of 1 mM or less. In one embodiment, the amphiphilic micelle forming agent contains one or more aliphatic chains with at least 11 carbon atoms and a headgroup, wherein the amphiphilic micelle forming agent is preferably selected from a molecule comprising or consisting of one or more C₆-C₂₀ long carbon chains conjugated to a linker that is further conjugated to a hydrophilic headgroup. In one embodiment, the linker is glycerol or one or more carbohydrates. In one embodiment, the hydrophilic headgroup is PEG, one or more carbohydrates, one or more hydrophilic polymers, or any combination thereof.

In one embodiment, CMPD 5 is co-formulated with a further amphiphilic organic compound of the present disclosure, including:
Poloxamers such as poloxamer 407, poloxamer 124, poloxamer f127, and poloxamer 188.
Tritons, such as Triton X-100, Triton X-114, Nonidet P-40 (NP-40), Igepal® CA-630
Glycosidic surfactant such as n-dodecyl-β-D-maltoside (DDM), beta-decyl-maltoside, octyl glucoside.
Tweens such as Tween-20, Tween-40, Tween 60, and Tween-80.

### CMPD 5 Micelle

In some embodiments, the micelle compositions of the present disclosure comprise the TLR7 agonist, CMPD 5. In one embodiment, the diameter of the micelle disclosed herein with CMPD 5 is between 5 nm and 50 nm, such as between 6 and 46 nm, such as between 7 and 42 nm, such as between 8 and 38 nm, such as between 9 and 34 nm, such as between 10 and 34 nm, such as between 11 nm and 30 nm, such as between 12 nm and 26 nm.

In one embodiment, the diameter of the micelle with CMPD 5 is from 5 nm to 39 nm, such as from 5 nm to 20 nm, such as from 20 nm to 30 nm, such as from 30 nm to 35 nm, such as from 35 nm to 39 nm.

In one embodiment, the diameter of the micelle is from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm.

In one embodiment, the diameter of the micelle with CMPD 5 is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

In one embodiment, the composition according to the present disclosure is provided which is a micelle composition, comprising an amphiphilic micelle-forming agent and CMPD 5. In one embodiment, the amphiphilic micelle-forming agent comprises one or more alkyl chains that are C8-C24 alkyl(s), such as C10-C22, such as C12-C20, preferably C14-C18, most preferred C16-C18 saturated alkyl chains or unsaturated alkyl chains.

In one embodiment, the micelle composition comprising CMPD 5 according to the present disclosure is provided, comprising a phospholipid, wherein the phospholipid comprises a phosphatidylethanolamine (PE), a phosphatidylcholine (PC), a phosphatidylserine (PS), a phosphatidylglycerol (PG), a phosphatidylinositol (PI), a phosphatidic acid (PA), a bisphosphatidyl glycerol (DPG), or a phosphatidyl alcohol.

In one embodiment, the phosphatidylethanolamine is selected from the group consisting of 1,2-dioleoyl-phosphatidylethanolamine, 1,2-dipalmitoyl-phosphatidylethanolamine, 1,2-dimyristoyl-phosphatidylethanolamine, 1,2-distearoyl-phosphatidylethanolamine, 1-oleoyl-2-palmitoyl-phosphatidylethanolamine, 1-oleoyl-2-stearoyl-phosphatidylethanolamine, 1-palmitoyl-2-oleoyl-phosphatidylethanolamine, and 1-stearoyl-2-oleoyl-phosphatidylethanolamine.

In one embodiment, the phospholipid conjugated to PEG is selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one embodiment, a micelle composition comprising CMPD 5 is provided comprising a phospholipid conjugated to PEG, wherein the phospholipid is: 1,2-distearoyl-sn-glycero-3-phosphoethanolamine (DSPE)-PEG. In one embodiment, the amphiphilic micelle-forming agent is DSPE-PEG2000.

### CMPD 5 Liposome

In one embodiment, a liposome is provided comprising CMPD 5, and a phospholipid. In one embodiment, a liposome is provided comprising CMPD 5, and a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine. In one embodiment, the liposome comprising CMPD 5 is provided wherein the phospholipid comprises alkyl chains that are C8-C24, preferably C10-C22, more preferred C12-C20, preferably C14-C18, most preferred C16-C18 saturated chains or unsaturated chains and wherein the amount of CMPD 5 is between 1-99%.

In one embodiment, a liposome is provided comprising CMPD 5, and a phospholipid conjugated to PEG. In one embodiment, a liposome is provided comprising CMPD 5, and a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one preferred embodiment of the present disclosure the liposome has 1-25% molar concentration of CMPD 5 relative to the phospholipid concentration, wherein CMPD 5 is embedded in the phospholipid containing part of the liposome.

In one embodiment, the liposome is provided, wherein the liposome comprises between 1% and 25% molar concentration of CMPD 5, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### CMPD 5 Nanodisc

In one embodiment, a nanodisc is provided comprising CMPD 5, a phospholipid, and a membrane scaffolding protein (MSP).

In one embodiment, a nanodisc is provided comprising CMPD 5, a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine, and a membrane scaffolding protein (MSP). In one embodiment, the nanodisc comprising CMPD 5 has a diameter from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm. In one embodiment, the diameter of this nanodisc is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

In one embodiment, a nanodisc is provided comprising CMPD 5, a phospholipid conjugated to PEG, and a membrane scaffolding protein (MSP).

In some embodiments, the nanodiscs of the present disclosure comprise CMPD 5 and one or more phospholipids selected from a group consisting of phosphatidylcholine (PC), phosphatidylethanolamine (PE), phosphatidylserine (PS), phosphatidylglycerol (PG), phosphatidylinositol (PI), phosphatidic acid (PA), DPG (bisphosphatidyl glycerol), and PEOH (phosphatidyl alcohol). In one embodiment, the nanodisc further comprises cholesterol, ergosterol and/or lanosterol.

In one embodiment, a nanodisc is provided comprising CMPD 5, a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG, and a membrane scaffolding protein (MSP).

In one preferred embodiment of the present disclosure the nanodisc has 1-25% molar concentration of CMPD 5 relative to the phospholipid concentration, wherein CMPD 5 is embedded in the phospholipid containing part of the nanodisc.

In one embodiment, the nanodisc is provided, wherein the composition comprises between 1% and 25% molar concentration of CMPD 5, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

In one embodiment, the nanodisc comprising CMPD 5 is a lipoprotein which can either be a High Density Lipoprotein (HDL), Low Density Lipoprotein (LDL) or Very Low Density Lipoprotein (VLDL). The lipoproteins comprising CMPD 5 may be either naturally occurring particles or synthetic produced particles. Naturally occurring particles may be derived from blood or plasma from patients or healthy subjects or from cell culture systems. Lipoprotein particles can be fused with CMPD 5 in organic solvent or as a particle and injected into patients.

### CMPD 5 Lipoplex

In one embodiment, a Lipoplex of CMPD 5 is provided. In one embodiment, the lipoplex of CMPD 5 comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipoplex of CMPD 5 also comprises cholesterol. In one embodiment, the Lipoplex of CMPD 5 also comprises a charged lipid, a nucleic acid sequence, such as DNA or RNA or mRNA or a modified nucleic acid sequence, or any combination thereof. In one embodiment, the Lipoplex is provided, wherein the composition comprises between 1% and 25% molar concentration of CMPD 5, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### CMPD 5 Lipidoid

In one embodiment, a Lipidoid of CMPD 5 is provided. In one embodiment, the Lipidoid of CMPD 5 comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipidoid of CMPD 5 also comprises cholesterol. In one embodiment, the Lipidoid of CMPD 5 also comprises a charged lipid, a nucleic acid sequence, such as DNA or RNA or mRNA or a modified nucleic acid sequence, or any combination thereof. In one embodiment, the Lipidoid is provided, wherein the composition comprises between 1% and 25% molar concentration of CMPD 5, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### Emulsions of CMPD 5

In one embodiment of the present disclosure, an emulsion is provided containing a lipid component and an aqueous component in which the lipid component partly contains amounts of CMPD 5 between 1-99%. An emulsion of CMPD 5 may preferably be used for intramuscular or subcutaneous administration. In one embodiment, the emulsion of the present disclosure comprising CMPD 5 also contains one or more emulsifiers.

### TLR7-Agonist: Telratolimod

In one embodiment, a composition is provided comprising the TLR7 agonist Telratolimod and one or more amphiphilic micelle forming agents. In one embodiment, the TLR7 agonist is Telratolimod,

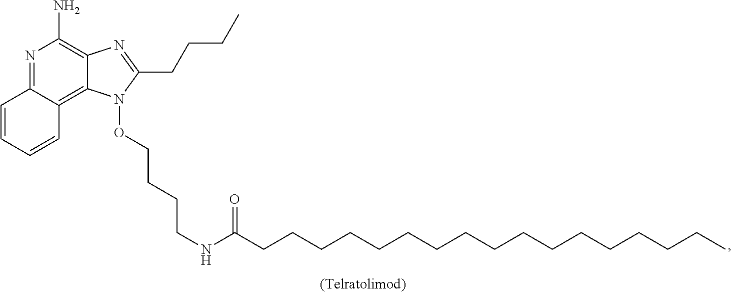 [SMILES: CCCCCCCCCCCCCCCCCC(=O)NCCCCOn1c(CCCC)nc2c(N)nc3ccccc3c21]

or a pharmaceutically acceptable salt thereof.

The TLR7 agonist Telratolimod can be co-formulated with one or more amphiphilic micelle forming agents. These amphiphilic micelle forming agents of the present disclosure have a hydrophobic tail and a hydrophilic headgroup providing a critical micelle concentration of 1 mM or less. In one embodiment, the amphiphilic micelle forming agent contains one or more aliphatic chains with at least 11 carbon atoms and a headgroup, wherein the amphiphilic micelle forming agent is preferably selected from a molecule comprising or consisting of one or more C6-C20 long carbon chains conjugated to a linker that is further conjugated to a hydrophilic headgroup. In one embodiment, the linker is glycerol or one or more carbohydrates. In one embodiment, the hydrophilic headgroup is PEG, one or more carbohydrates, one or more hydrophilic polymers, or any combination thereof.

In one embodiment, Telratolimod is co-formulated with a further amphiphilic organic compound of the present disclosure, including:
Poloxamers such as poloxamer 407, poloxamer 124, poloxamer f127, and poloxamer 188.
Tritons, such as Triton X-100, Triton X-114, Nonidet P-40 (NP-40), Igepal® CA-630
Glycosidic surfactant such as n-dodecyl-β-D-maltoside (DDM), beta-decyl-maltoside, octyl glucoside.
Tweens such as Tween-20, Tween-40, Tween 60, and Tween-80.

### Telratolimod Micelle

In some embodiments, the micelle compositions of the present disclosure comprise the TLR7 agonist, Telratolimod. In one embodiment, the diameter of the micelle disclosed herein with Telratolimod is between 5 nm and 50 nm, such as between 6 and 46 nm, such as between 7 and 42 nm, such as between 8 and 38 nm, such as between 9 and 34 nm, such as between 10 and 34 nm, such as between 11 nm and 30 nm, such as between 12 nm and 26 nm.

In one embodiment, the diameter of the micelle with Telratolimod is from 5 nm to 39 nm, such as from 5 nm to 20 nm, such as from 20 nm to 30 nm, such as from 30 nm to 35 nm, such as from 35 nm to 39 nm.

In one embodiment, the diameter of the micelle is from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm.

In one embodiment, the diameter of the micelle with Telratolimod is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

In one embodiment, the composition according to the present disclosure is provided which is a micelle composition, comprising an amphiphilic micelle-forming agent and Telratolimod. In one embodiment, the amphiphilic micelle-forming agent comprises one or more alkyl chains that are C8-C24 alkyl(s), such as C10-C22, such as C12-C20, preferably C14-C18, most preferred C16-C18 saturated alkyl chains or unsaturated alkyl chains.

In one embodiment, the micelle composition comprising Telratolimod according to the present disclosure is provided, comprising a phospholipid, wherein the phospholipid comprises a phosphatidylethanolamine (PE), a phosphatidylcholine (PC), a phosphatidylserine (PS), a phosphatidylglycerol (PG), a phosphatidylinositol (PI), a phosphatidic acid (PA), a bisphosphatidyl glycerol (DPG), or a phosphatidyl alcohol.

In one embodiment, the phosphatidylethanolamine is selected from the group consisting of 1,2-dioleoyl-phosphatidylethanolamine, 1,2-dipalmitoyl-phosphatidylethanolamine, 1,2-dimyristoyl-phosphatidylethanolamine, 1,2-distearoyl-phosphatidylethanolamine, 1-oleoyl-2-palmitoyl-phosphatidylethanolamine, 1-oleoyl-2-stearoyl-phosphatidylethanolamine, 1-palmitoyl-2-oleoyl-phosphatidylethanolamine, and 1-stearoyl-2-oleoyl-phosphatidylethanolamine.

In one embodiment, the phospholipid conjugated to PEG is selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one embodiment, a micelle composition comprising Telratolimod is provided comprising a phospholipid conjugated to PEG, wherein the phospholipid is: 1,2-distearoyl-sn-glycero-3-phosphoethanolamine (DSPE)-PEG. In one embodiment, the amphiphilic micelle-forming agent is DSPE-PEG2000.

### Telratolimod Liposome

In one embodiment, a liposome is provided comprising Telratolimod, and a phospholipid.

In one embodiment, a liposome is provided comprising Telratolimod, and a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine. In one embodiment, the liposome comprising Telratolimod is provided wherein the phospholipid comprises alkyl chains that are C8-C24, preferably C10-C22, more preferred C12-C20, preferably C14-C18, most preferred C16-C18 saturated chains or unsaturated chains and wherein the amount of Telratolimod is between 1-99%.

In one embodiment, a liposome is provided comprising Telratolimod, and a phospholipid conjugated to PEG. In one embodiment, a liposome is provided comprising Telratolimod, and a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one preferred embodiment of the present disclosure the liposome has 1-25% molar concentration of Telratolimod relative to the phospholipid concentration, wherein Telratolimod is embedded in the phospholipid containing part of the liposome.

In one embodiment, the liposome is provided, wherein the liposome comprises between 1% and 25% molar concentration of Telratolimod, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### Telratolimod Nanodisc

In one embodiment, a nanodisc is provided comprising Telratolimod, a phospholipid, and a membrane scaffolding protein (MSP).

In one embodiment, a nanodisc is provided comprising Telratolimod, a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine, and a membrane scaffolding protein (MSP). In one embodiment, the nanodisc comprising Telratolimod has a diameter from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm. In one embodiment, the diameter of this nanodisc is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

In one embodiment, a nanodisc is provided comprising Telratolimod, a phospholipid conjugated to PEG, and a membrane scaffolding protein (MSP).

In some embodiments, the nanodiscs of the present disclosure comprise Telratolimod and one or more phospholipids selected from a group consisting of phosphatidylcholine (PC), phosphatidylethanolamine (PE), phosphatidylserine (PS), phosphatidylglycerol (PG), phosphatidylinositol (PI), phosphatidic acid (PA), DPG (bisphosphatidyl glycerol), and PEOH (phosphatidyl alcohol). In one embodiment, the nanodisc further comprises cholesterol, ergosterol and/or lanosterol.

In one embodiment, a nanodisc is provided comprising Telratolimod, a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG, and a membrane scaffolding protein (MSP).

In one preferred embodiment of the present disclosure the nanodisc has 1-25% molar concentration of Telratolimod relative to the phospholipid concentration, wherein Telratolimod is embedded in the phospholipid containing part of the nanodisc.

In one embodiment, the nanodisc is provided, wherein the composition comprises between 1% and 25% molar concentration of Telratolimod, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

In one embodiment, the nanodisc comprising Telratolimod is a lipoprotein which can either be a High Density Lipoprotein (HDL), Low Density Lipoprotein (LDL) or Very Low Density Lipoprotein (VLDL). The lipoproteins comprising Telratolimod may be either naturally occurring particles or synthetic produced particles. Naturally occurring particles may be derived from blood or plasma from patients or healthy subjects or from cell culture systems. Lipoprotein particles can be fused with Telratolimod in organic solvent or as a particle and injected into patients.

### Telratolimod Lipoplex

In one embodiment, a Lipoplex of Telratolimod is provided. In one embodiment, the lipoplex of Telratolimod comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipoplex of Telratolimod also comprises cholesterol. In one embodiment, the Lipoplex of Telratolimod also comprises a charged lipid, a nucleic acid sequence, such as DNA or RNA or mRNA or a modified nucleic acid sequence, or any combination thereof. In one embodiment, the Lipoplex is provided, wherein the composition comprises between 1% and 25% molar concentration of Telratolimod, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### Telratolimod Lipidoid

In one embodiment, a Lipidoid of Telratolimod is provided. In one embodiment, the Lipidoid of Telratolimod comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipidoid of Telratolimod also comprises cholesterol. In one embodiment, the Lipidoid of Telratolimod also comprises a charged lipid, a nucleic acid sequence, such as DNA or RNA or mRNA or a modified nucleic acid sequence, or any combination thereof. In one embodiment, the Lipidoid is provided, wherein the composition comprises between 1% and 25% molar concentration of Telratolimod, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### Emulsions of Telratolimod

In one embodiment of the present disclosure, an emulsion is provided containing a lipid component and an aqueous component in which the lipid component partly contains amounts of Telratolimod between 1-99%. An emulsion of may preferably be used for intramuscular or subcutaneous administration. In one embodiment, the emulsion of the present disclosure comprising Telratolimod also contains one or more emulsifiers.

TLR7-Agonist: CL347 In one embodiment, a composition is provided comprising the TLR7 agonist CL347 and one or more amphiphilic micelle forming agents In one embodiment, the TLR7 agonist is CL347,

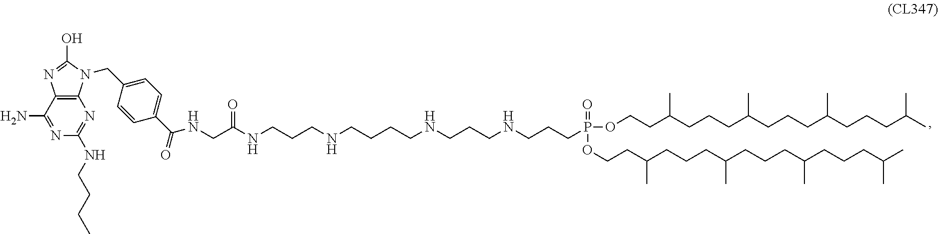 [SMILES: CCCCNc1nc(N)c2nc(O)n(Cc3ccc(C(=O)NCC(=O)NCCCNCCCCNCCCNCCCP(=O)(OCCC(C)CCCC(C)CCCC(C)CCCC(C)C)OCCC(C)CCCC(C)CCCC(C)CCCC(C)C)cc3)c2n1]

or a pharmaceutically acceptable salt thereof.

The TLR7 agonist CL347 can be co-formulated with one or more amphiphilic micelle forming agents. These amphiphilic micelle forming agents of the present disclosure have a hydrophobic tail and a hydrophilic headgroup providing a critical micelle concentration of 1 mM or less. In one embodiment, the amphiphilic micelle forming agent contains one or more aliphatic chains with at least 11 carbon atoms and a headgroup, wherein the amphiphilic micelle forming agent is preferably selected from a molecule comprising or consisting of one or more C₆-C₂₀ long carbon chains conjugated to a linker that is further conjugated to a hydrophilic headgroup. In one embodiment, the linker is glycerol or one or more carbohydrates. In one embodiment, the hydrophilic headgroup is PEG, one or more carbohydrates, one or more hydrophilic polymers, or any combination thereof.

In one embodiment, CL347 is co-formulated with a further amphiphilic organic compound of the present disclosure, including:
Poloxamers such as poloxamer 407, poloxamer 124, poloxamer f127, and poloxamer 188.
Tritons, such as Triton X-100, Triton X-114, Nonidet P-40 (NP-40), Igepal® CA-630
Glycosidic surfactant such as n-dodecyl-β-D-maltoside (DDM), beta-decyl-maltoside, octyl glucoside.
Tweens such as Tween-20, Tween-40, Tween 60, and Tween-80.

### CL347 Micelle

In some embodiments, the micelle compositions of the present disclosure comprise the TLR7 agonist, CL347. In one embodiment, the diameter of the micelle disclosed herein with CL347 is between 5 nm and 50 nm, such as between 6 and 46 nm, such as between 7 and 42 nm, such as between 8 and 38 nm, such as between 9 and 34 nm, such as between 10 and 34 nm, such as between 11 nm and 30 nm, such as between 12 nm and 26 nm.

In one embodiment, the diameter of the micelle with CL347 is from 5 nm to 39 nm, such as from 5 nm to 20 nm, such as from 20 nm to 30 nm, such as from 30 nm to 35 nm, such as from 35 nm to 39 nm.

In one embodiment, the diameter of the micelle is from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm.

In one embodiment, the diameter of the micelle with CL347 is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

In one embodiment, the composition according to the present disclosure is provided which is a micelle composition, comprising an amphiphilic micelle-forming agent and CL347. In one embodiment, the amphiphilic micelle-forming agent comprises one or more alkyl chains that are C8-C24 alkyl(s), such as C10-C22, such as C12-C20, preferably C14-C18, most preferred C16-C18 saturated alkyl chains or unsaturated alkyl chains.

In one embodiment, the micelle composition comprising CL347 according to the present disclosure is provided, comprising a phospholipid, wherein the phospholipid comprises a phosphatidylethanolamine (PE), a phosphatidylcholine (PC), a phosphatidylserine (PS), a phosphatidylglycerol (PG), a phosphatidylinositol (PI), a phosphatidic acid (PA), a bisphosphatidyl glycerol (DPG), or a phosphatidyl alcohol.

In one embodiment, the phosphatidylethanolamine is selected from the group consisting of 1,2-dioleoyl-phosphatidylethanolamine, 1,2-dipalmitoyl-phosphatidylethanolamine, 1,2-dimyristoyl-phosphatidylethanolamine, 1,2-distearoyl-phosphatidylethanolamine, 1-oleoyl-2-palmitoyl-phosphatidylethanolamine, 1-oleoyl-2-stearoyl-phosphatidylethanolamine, 1-palmitoyl-2-oleoyl-phosphatidylethanolamine, and 1-stearoyl-2-oleoyl-phosphatidylethanolamine.

In one embodiment, the phospholipid conjugated to PEG is selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one embodiment, a micelle composition comprising CL347 is provided comprising a phospholipid conjugated to PEG, wherein the phospholipid is: 1,2-distearoyl-sn-glycero-3-phosphoethanolamine (DSPE)-PEG. In one embodiment, the amphiphilic micelle-forming agent is DSPE-PEG2000.

### CL347 Liposome

In one embodiment, a liposome is provided comprising CL347, and a phospholipid.

In one embodiment, a liposome is provided comprising CL347, and a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine. In one embodiment, the liposome comprising CL347 is provided wherein the phospholipid comprises alkyl chains that are C8-C24, preferably C10-C22, more preferred C12-C20, preferably C14-C18, most preferred C16-C18 saturated chains or unsaturated chains and wherein the amount of CL347 is between 1-99%.

In one embodiment, a liposome is provided comprising CL347, and a phospholipid conjugated to PEG. In one embodiment, a liposome is provided comprising CL347, and a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one preferred embodiment of the present disclosure the liposome has 1-25% molar concentration of CL347 relative to the phospholipid concentration, wherein CL347 is embedded in the phospholipid containing part of the liposome.

In one embodiment, the liposome is provided, wherein the liposome comprises between 1% and 25% molar concentration of CL347, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### CL347 Nanodisc

In one embodiment, a nanodisc is provided comprising CL347, a phospholipid, and a membrane scaffolding protein (MSP).

In one embodiment, a nanodisc is provided comprising CL347, a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine, and a membrane scaffolding protein (MSP). In one embodiment, the nanodisc comprising CL347 has a diameter from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm. In one embodiment, the diameter of this nanodisc is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

In one embodiment, a nanodisc is provided comprising CL347, a phospholipid conjugated to PEG, and a membrane scaffolding protein (MSP).

In some embodiments, the nanodiscs of the present disclosure comprise CL347 and one or more phospholipids selected from a group consisting of phosphatidylcholine (PC), phosphatidylethanolamine (PE), phosphatidylserine (PS), phosphatidylglycerol (PG), phosphatidylinositol (PI), phosphatidic acid (PA), DPG (bisphosphatidyl glycerol), and PEOH (phosphatidyl alcohol). In one embodiment, the nanodisc further comprises cholesterol, ergosterol and/or lanosterol.

In one embodiment, a nanodisc is provided comprising CL347, a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG, and a membrane scaffolding protein (MSP).

In one preferred embodiment of the present disclosure the nanodisc has 1-25% molar concentration of CL347 relative to the phospholipid concentration, wherein CL347 is embedded in the phospholipid containing part of the nanodisc.

In one embodiment, the nanodisc is provided, wherein the composition comprises between 1% and 25% molar concentration of CL347, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

In one embodiment, the nanodisc comprising CL347 is a lipoprotein which can either be a High Density Lipoprotein (HDL), Low Density Lipoprotein (LDL) or Very Low Density Lipoprotein (VLDL). The lipoproteins comprising CL347 may be either naturally occurring particles or synthetic produced particles. Naturally occurring particles may be derived from blood or plasma from patients or healthy subjects or from cell culture systems. Lipoprotein particles can be fused with CL347 in organic solvent or as a particle and injected into patients.

### CL347 Lipoplex

In one embodiment, a Lipoplex of CL347 is provided. In one embodiment, the lipoplex of CL347 comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipoplex of CL347 also comprises cholesterol. In one embodiment, the Lipoplex of CL347 also comprises a charged lipid, a nucleic acid sequence, such as DNA or RNA or mRNA or a modified nucleic acid sequence, or any combination thereof. In one embodiment, the Lipoplex is provided, wherein the composition comprises between 1% and 25% molar concentration of CL347, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### CL347 Lipidoid

In one embodiment, a Lipidoid of CL347 is provided. In one embodiment, the Lipidoid of CL347 comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipidoid of CL347 also comprises cholesterol. In one embodiment, the Lipidoid of CL347 also comprises a charged lipid, a nucleic acid sequence, such as DNA or RNA or mRNA or a modified nucleic acid sequence, or any combination thereof. In one embodiment, the Lipidoid is provided, wherein the composition comprises between 1% and 25% molar concentration of CL347, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### Emulsions of CL347

In one embodiment of the present disclosure, an emulsion is provided containing a lipid component and an aqueous component in which the lipid component partly contains amounts of CL347 between 1-99%. An emulsion of may preferably be used for intramuscular or subcutaneous administration. In one embodiment, the emulsion of the present disclosure comprising CL347 also contains one or more emulsifiers.

### TLR7-Agonist: CL413

In one embodiment, a composition is provided comprising the TLR7 agonist CL413 and one or more amphiphilic micelle forming agents. In one embodiment, the TLR7 agonist is CL413,

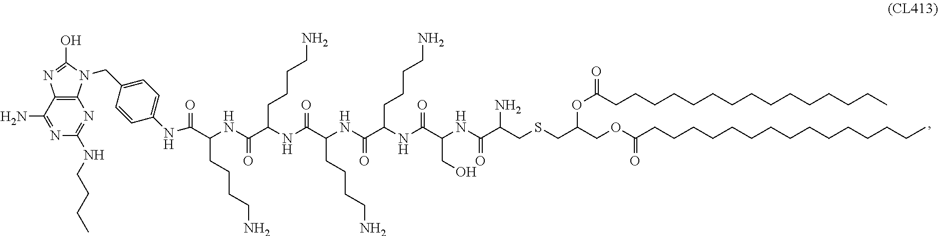 [SMILES: CCCCCCCCCCCCCCCC(=O)OCC(CSCC(N)C(=O)NC(CO)C(=O)NC(CCCCN)C(=O)NC(CCCCN)C(=O)NC(CCCCN)C(=O)NC(CCCCN)C(=O)Nc1ccc(Cn2c(O)nc3c(N)nc(NCCCC)nc32)cc1)OC(=O)CCCCCCCCCCCCCCC]

or a pharmaceutically acceptable salt thereof.

The TLR7 agonist CL413 can be co-formulated with one or more amphiphilic micelle forming agents. These amphiphilic micelle forming agents of the present disclosure have a hydrophobic tail and a hydrophilic headgroup providing a critical micelle concentration of 1 mM or less. In one embodiment, the amphiphilic micelle forming agent contains one or more aliphatic chains with at least 11 carbon atoms and a headgroup, wherein the amphiphilic micelle forming agent is preferably selected from a molecule comprising or consisting of one or more C6-C20 long carbon chains conjugated to a linker that is further conjugated to a hydrophilic headgroup. In one embodiment, the linker is glycerol or one or more carbohydrates. In one embodiment, the hydrophilic headgroup is PEG, one or more carbohydrates, one or more hydrophilic polymers, or any combination thereof.

In one embodiment, CL413 is co-formulated with a further amphiphilic organic compound of the present disclosure, including:
Poloxamers such as poloxamer 407, poloxamer 124, poloxamer f127, and poloxamer 188.
Tritons, such as Triton X-100, Triton X-114, Nonidet P-40 (NP-40), Igepal® CA-630
Glycosidic surfactant such as n-dodecyl-β-D-maltoside (DDM), beta-decyl-maltoside, octyl glucoside.
Tweens such as Tween-20, Tween-40, Tween 60, and Tween-80.

### CL413 Micelle

In some embodiments, the micelle compositions of the present disclosure comprise the TLR7 agonist, CL413. In one embodiment, the diameter of the micelle disclosed herein with CL413 is between 5 nm and 50 nm, such as between 6 and 46 nm, such as between 7 and 42 nm, such as between 8 and 38 nm, such as between 9 and 34 nm, such as between 10 and 34 nm, such as between 11 nm and 30 nm, such as between 12 nm and 26 nm.

In one embodiment, the diameter of the micelle with CL413 is from 5 nm to 39 nm, such as from 5 nm to 20 nm, such as from 20 nm to 30 nm, such as from 30 nm to 35 nm, such as from 35 nm to 39 nm.

In one embodiment, the diameter of the micelle is from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm.

In one embodiment, the diameter of the micelle with CL413 is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

In one embodiment, the composition according to the present disclosure is provided which is a micelle composition, comprising an amphiphilic micelle-forming agent and CL413. In one embodiment, the amphiphilic micelle-forming agent comprises one or more alkyl chains that are C8-C24 alkyl(s), such as C10-C22, such as C12-C20, preferably C14-C18, most preferred C16-C18 saturated alkyl chains or unsaturated alkyl chains.

In one embodiment, the micelle composition comprising CL413 according to the present disclosure is provided, comprising a phospholipid, wherein the phospholipid comprises a phosphatidylethanolamine (PE), a phosphatidylcholine (PC), a phosphatidylserine (PS), a phosphatidylglycerol (PG), a phosphatidylinositol (PI), a phosphatidic acid (PA), a bisphosphatidyl glycerol (DPG), or a phosphatidyl alcohol.

In one embodiment, the phosphatidylethanolamine is selected from the group consisting of 1,2-dioleoyl-phosphatidylethanolamine, 1,2-dipalmitoyl-phosphatidylethanolamine, 1,2-dimyristoyl-phosphatidylethanolamine, 1,2-distearoyl-phosphatidylethanolamine, 1-oleoyl-2-palmitoyl-phosphatidylethanolamine, 1-oleoyl-2-stearoyl-phosphatidylethanolamine, 1-palmitoyl-2-oleoyl-phosphatidylethanolamine, and 1-stearoyl-2-oleoyl-phosphatidylethanolamine.

In one embodiment, the phospholipid conjugated to PEG is selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one embodiment, a micelle composition comprising CL413 is provided comprising a phospholipid conjugated to PEG, wherein the phospholipid is: 1,2-distearoyl-sn-glycero-3-phosphoethanolamine (DSPE)-PEG. In one embodiment, the amphiphilic micelle-forming agent is DSPE-PEG2000.

### CL413 Liposome

In one embodiment, a liposome is provided comprising CL413, and a phospholipid.

In one embodiment, a liposome is provided comprising CL413, and a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine. In one embodiment, the liposome comprising CL413 is provided wherein the phospholipid comprises alkyl chains that are C8-C24, preferably C10-C22, more preferred C12-C20, preferably C14-C18, most preferred C16-C18 saturated chains or unsaturated chains and wherein the amount of CL413 is between 1-99%. In one embodiment, a liposome is provided comprising CL413, and a phospholipid conjugated to PEG. In one embodiment, a liposome is provided comprising CL413, and a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one preferred embodiment of the present disclosure the liposome has 1-25% molar concentration of CL413 relative to the phospholipid concentration, wherein CL413 is embedded in the phospholipid containing part of the liposome.

In one embodiment, the liposome is provided, wherein the liposome comprises between 1% and 25% molar concentration of CL413, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### CL413 Nanodisc

In one embodiment, a nanodisc is provided comprising CL413, a phospholipid, and a membrane scaffolding protein (MSP).

In one embodiment, a nanodisc is provided comprising CL413, a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine, and a membrane scaffolding protein (MSP). In one embodiment, the nanodisc comprising CL413 has a diameter from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm. In one embodiment, the diameter of this nanodisc is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

In one embodiment, a nanodisc is provided comprising CL413, a phospholipid conjugated to PEG, and a membrane scaffolding protein (MSP).

In some embodiments, the nanodiscs of the present disclosure comprise CL413 and one or more phospholipids selected from a group consisting of phosphatidylcholine (PC), phosphatidylethanolamine (PE), phosphatidylserine (PS), phosphatidylglycerol (PG), phosphatidylinositol (PI), phosphatidic acid (PA), DPG (bisphosphatidyl glycerol), and PEOH (phosphatidyl alcohol). In one embodiment, the nanodisc further comprises cholesterol, ergosterol and/or lanosterol.

In one embodiment, a nanodisc is provided comprising CL413, a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG, and a membrane scaffolding protein (MSP).

In one preferred embodiment of the present disclosure the nanodisc has 1-25% molar concentration of CL413 relative to the phospholipid concentration, wherein CL413 is embedded in the phospholipid containing part of the nanodisc.

In one embodiment, the nanodisc is provided, wherein the nanodisc comprises between 1% and 25% molar concentration of CL413, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

In one embodiment, the nanodisc comprising CL413 is a lipoprotein which can either be a High Density Lipoprotein (HDL), Low Density Lipoprotein (LDL) or Very Low Density Lipoprotein (VLDL). The lipoproteins comprising CL413 may be either naturally occurring particles or synthetic produced particles. Naturally occurring particles may be derived from blood or plasma from patients or healthy subjects or from cell culture systems. Lipoprotein particles can be fused with CL413 in organic solvent or as a particle and injected into patients.

### CL413 Lipoplex

In one embodiment, a Lipoplex of CL413 is provided. In one embodiment, the lipoplex of CL413 comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipoplex of CL413 also comprises cholesterol. In one embodiment, the Lipoplex of CL413 also comprises a charged lipid, a nucleic acid sequence, such as DNA or RNA or mRNA or a modified nucleic acid sequence, or any combination thereof. In one embodiment, the Lipoplex is provided, wherein the composition comprises between 1% and 25% molar concentration of CL413, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### CL413 Lipidoid

In one embodiment, a Lipidoid of CL413 is provided. In one embodiment, the Lipidoid of CL413 comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipidoid of CL413 also comprises cholesterol. In one embodiment, the Lipidoid of CL413 also comprises a charged lipid, a nucleic acid sequence, such as DNA or RNA or mRNA or a modified nucleic acid sequence, or any combination thereof. In one embodiment, the Lipidoid is provided, wherein the composition comprises between 1% and 25% molar concentration of CL413, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### Emulsions of CL413

In one embodiment of the present disclosure, an emulsion is provided containing a lipid component and an aqueous component in which the lipid component partly contains amounts of CL413 between 1-99%. An emulsion of may preferably be used for intramuscular or subcutaneous administration. In one embodiment, the emulsion of the present disclosure comprising CL413 also contains one or more emulsifiers.

### TLR7-Agonist: CL531

In one embodiment, a composition is provided comprising the TLR7 agonist CL531 and one or more amphiphilic micelle forming agents. In one embodiment, the TLR7 agonist is CL531,

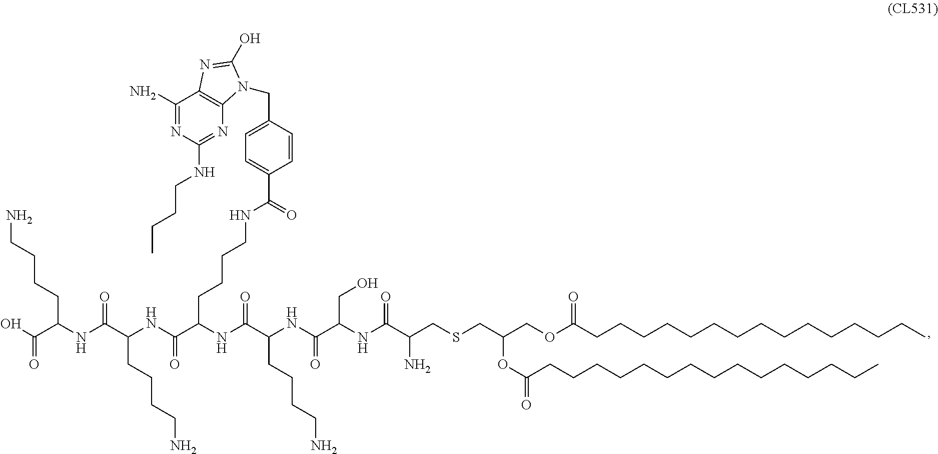 [SMILES: CCCCCCCCCCCCCCCC(=O)OCC(CSCC(N)C(=O)NC(CO)C(=O)NC(CCCCN)C(=O)NC(CCCCNC(=O)c1ccc(Cn2c(O)nc3c(N)nc(NCCCC)nc32)cc1)C(=O)NC(CCCCN)C(=O)NC(CCCCN)C(=O)O)OC(=O)CCCCCCCCCCCCCCC]

or a pharmaceutically acceptable salt thereof.

The TLR7 agonist CL531 can be co-formulated with one or more amphiphilic micelle forming agents. These amphiphilic micelle forming agents of the present disclosure have a hydrophobic tail and a hydrophilic headgroup providing a critical micelle concentration of 1 mM or less. In one embodiment, the amphiphilic micelle forming agent contains one or more aliphatic chains with at least 11 carbon atoms and a headgroup, wherein the amphiphilic micelle forming agent is preferably selected from a molecule comprising or consisting of one or more C6-C20 long carbon chains conjugated to a linker that is further conjugated to a hydrophilic headgroup. In one embodiment, the linker is glycerol or one or more carbohydrates. In one embodiment, the hydrophilic headgroup is PEG, one or more carbohydrates, one or more hydrophilic polymers, or any combination thereof.

In one embodiment, CL531 is co-formulated with a further amphiphilic organic compound of the present disclosure, including:
Poloxamers such as poloxamer 407, poloxamer 124, poloxamer f127, and poloxamer 188.
Tritons, such as Triton X-100, Triton X-114, Nonidet P-40 (NP-40), Igepal® CA-630
Glycosidic surfactant such as n-dodecyl-β-D-maltoside (DDM), beta-decyl-maltoside, octyl glucoside.
Tweens such as Tween-20, Tween-40, Tween 60, and Tween-80.

### CL531 Micelle

In some embodiments, the micelle compositions of the present disclosure comprise the TLR7 agonist, CL531. In one embodiment, the diameter of the micelle disclosed herein with CL531 is between 5 nm and 50 nm, such as between 6 and 46 nm, such as between 7 and 42 nm, such as between 8 and 38 nm, such as between 9 and 34 nm, such as between 10 and 34 nm, such as between 11 nm and 30 nm, such as between 12 nm and 26 nm.

In one embodiment, the diameter of the micelle with CL531 is from 5 nm to 39 nm, such as from 5 nm to 20 nm, such as from 20 nm to 30 nm, such as from 30 nm to 35 nm, such as from 35 nm to 39 nm.

In one embodiment, the diameter of the micelle is from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm.

In one embodiment, the diameter of the micelle with CL531 is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

In one embodiment, the composition according to the present disclosure is provided which is a micelle composition, comprising an amphiphilic micelle-forming agent and CL531. In one embodiment, the amphiphilic micelle-forming agent comprises one or more alkyl chains that are C8-C24 alkyl(s), such as C10-C22, such as C12-C20, preferably C14-C18, most preferred C16-C18 saturated alkyl chains or unsaturated alkyl chains.

In one embodiment, the micelle composition comprising CL531 according to the present disclosure is provided, comprising a phospholipid, wherein the phospholipid comprises a phosphatidylethanolamine (PE), a phosphatidylcholine (PC), a phosphatidylserine (PS), a phosphatidylglycerol (PG), a phosphatidylinositol (PI), a phosphatidic acid (PA), a bisphosphatidyl glycerol (DPG), or a phosphatidyl alcohol.

In one embodiment, the phosphatidylethanolamine is selected from the group consisting of 1,2-dioleoyl-phosphatidylethanolamine, 1,2-dipalmitoyl-phosphatidylethanolamine, 1,2-dimyristoyl-phosphatidylethanolamine, 1,2-distearoyl-phosphatidylethanolamine, 1-oleoyl-2-palmitoyl-phosphatidylethanolamine, 1-oleoyl-2-stearoyl-phosphatidylethanolamine, 1-palmitoyl-2-oleoyl-phosphatidylethanolamine, and 1-stearoyl-2-oleoyl-phosphatidylethanolamine.

In one embodiment, the phospholipid conjugated to PEG is selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one embodiment, a micelle composition comprising CL531 is provided comprising a phospholipid conjugated to PEG, wherein the phospholipid is: 1,2-distearoyl-sn-glycero-3-phosphoethanolamine (DSPE)-PEG. In one embodiment, the amphiphilic micelle-forming agent is DSPE-PEG2000.

### CL531 Liposome

In one embodiment, a liposome is provided comprising CL531, and a phospholipid.

In one embodiment, a liposome is provided comprising CL531, and a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine. In one embodiment, the liposome comprising CL531 is provided wherein the phospholipid comprises alkyl chains that are C8-C24, preferably C10-C22, more preferred C12-C20, preferably C14-C18, most preferred C16-C18 saturated chains or unsaturated chains and wherein the amount of CL531 is between 1-99%.

In one embodiment, a liposome is provided comprising CL531, and a phospholipid conjugated to PEG. In one embodiment, a liposome is provided comprising CL531, and a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

In one preferred embodiment of the present disclosure the liposome has 1-25% molar concentration of CL531 relative to the phospholipid concentration, wherein CL531 is embedded in the phospholipid containing part of the liposome.

In one embodiment, the liposome is provided, wherein the liposome comprises between 1% and 25% molar concentration of CL531, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### CL531 Nanodisc

In one embodiment, a nanodisc is provided comprising CL531, a phospholipid, and a membrane scaffolding protein (MSP).

In one embodiment, a nanodisc is provided comprising CL531, a phospholipid selected from the group consisting of 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine, and a membrane scaffolding protein (MSP). In one embodiment, the nanodisc comprising CL531 has a diameter from 5 nm to 39 nm, such as from 5 nm to 38 nm, such as from 5 nm to 37 nm, such as from 5 nm to 36 nm, such as from 5 nm to 35 nm, such as from 5 nm to 34 nm, such as from 5 nm to 33 nm, such as from 5 nm to 32 nm, such as from 5 nm to 31 nm. In one embodiment, the diameter of this nanodisc is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

In one embodiment, a nanodisc is provided comprising CL531, a phospholipid conjugated to PEG, and a membrane scaffolding protein (MSP).

In some embodiments, the nanodiscs of the present disclosure comprise CL531 and one or more phospholipids selected from a group consisting of phosphatidylcholine (PC), phosphatidylethanolamine (PE), phosphatidylserine (PS), phosphatidylglycerol (PG), phosphatidylinositol (PI), phosphatidic acid (PA), DPG (bisphosphatidyl glycerol), and PEOH (phosphatidyl alcohol). In one embodiment, the nanodisc further comprises cholesterol, ergosterol and/or lanosterol.

In one embodiment, a nanodisc is provided comprising CL531, a phospholipid conjugated to PEG selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG, and a membrane scaffolding protein (MSP).

In one preferred embodiment of the present disclosure the nanodisc has 1-25% molar concentration of CL531 relative to the phospholipid concentration, wherein CL531 is embedded in the phospholipid containing part of the nanodisc.

In one embodiment, the nanodisc is provided, wherein the nanodisc comprises between 1% and 25% molar concentration of CL531, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

In one embodiment, the nanodisc comprising CL531 is a lipoprotein which can either be a High Density Lipoprotein (HDL), Low Density Lipoprotein (LDL) or Very Low Density Lipoprotein (VLDL). The lipoproteins comprising CL531 may be either naturally occurring particles or synthetic produced particles. Naturally occurring particles may be derived from blood or plasma from patients or healthy subjects or from cell culture systems. Lipoprotein particles can be fused with CL531 in organic solvent or as a particle and injected into patients.

### CL531 Lipoplex

In one embodiment, a Lipoplex of CL531 is provided. In one embodiment, the lipoplex of CL531 comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipoplex of CL531 also comprises cholesterol. In one embodiment, the Lipoplex of CL531 also comprises a charged lipid, a nucleic acid sequence, such as DNA or RNA or mRNA or a modified nucleic acid sequence, or any combination thereof. In one embodiment, the Lipoplex is provided, wherein the composition comprises between 1% and 25% molar concentration of CL531, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### CL531 Lipidoid

In one embodiment, a Lipidoid of CL531 is provided. In one embodiment, the Lipidoid of CL531 comprises a lipid selected from the group consisting of 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG. In one embodiment, the Lipidoid of CL531 also comprises cholesterol. In one embodiment, the Lipidoid of CL531 also comprises a charged lipid, a nucleic acid sequence, such as DNA or RNA or mRNA or a modified nucleic acid sequence, or any combination thereof. In one embodiment, the Lipidoid is provided, wherein the composition comprises between 1% and 25% molar concentration of CL531, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

### Emulsions of CL531

In one embodiment of the present disclosure, an emulsion is provided containing a lipid component and an aqueous component in which the lipid component partly contains amounts of CL531 between 1-99%. An emulsion of CL531 may preferably be used for intramuscular or subcutaneous administration. In one embodiment, the emulsion of the present disclosure comprising CL531 also contains one or more emulsifiers.

### Further Active Agents

In one embodiment, the micelle composition as defined herein is provided, further comprising at least one further active ingredient. In one embodiment, the micelle composition further comprises at least one antigen.

### Items

I-1. A micelle composition comprising:
   a toll-like receptor 7 (TLR7) agonist of formula (I), formula (II), formula (III) or formula (IV);

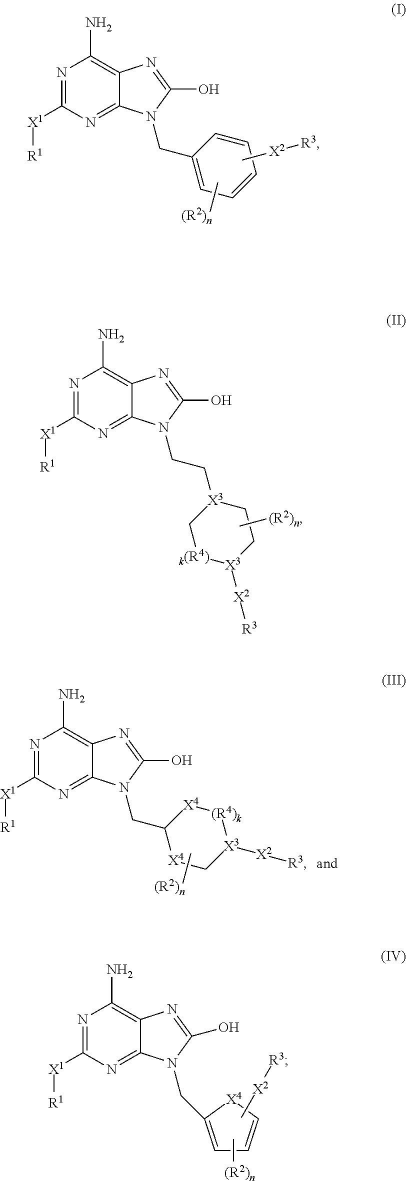

wherein X¹ is —O—, —S—, or —NR^{C};
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R^{C} is hydrogen, C₁₋₁₀alkyl, or substituted C₁₋₁₀alkyl; or R^{C} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent; each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxy C₁₋₆alkylene, amino, cyano, halo, or aryl;
n is 0, 1, 2, 3 or 4;
X² is a bond or a linking group; and
R³ is a lipid;
X³ is —N— or —CH—;
R⁴ is —CH₂— or —CH(R²)—; and
k is 0 or 1;
X⁴ is —O—, —S—, —NH—, —N(R^{d})—, —CH₂—, or —CH(R²)—;
each R^{d} is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)O(C₁-C₆)alkyl (alkoxycarbonyl), substituted —C(O)O(C₁-C₆)alkyl, —C(O)NR^{a}R^{b} (carbamoyl);
or a tautomer thereof;
or a pharmaceutically acceptable salt or solvate thereof, and wherein the ring system of formula (II) is a piperidine ring with one heteroatom being an N atom and with the N-atom of the piperidine ring adjacent to X², and wherein the purine group in any of Formula (I), (II), (III), or (IV) is subject to tautomeric rearrangements;
and an amphiphilic micelle-forming agent.
I-2. The micelle composition according to any one of the preceding items, wherein the TLR7 agonist is of formula (I):

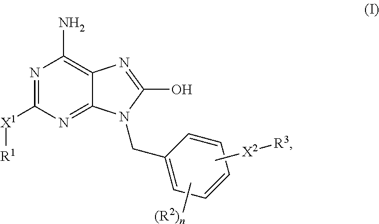

wherein X¹ is —O—, —S—, or —NR^{C};
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R¹ is hydrogen, C₁₋₁₀alkyl, or substituted C₁₋₁₀alkyl; or R^{C} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent; each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxy C₁₋₆alkylene, amino, cyano, halo, or aryl;
n is 0, 1, or 2;
X² is a bond or a linking group; and
R³ is a lipid;
or a tautomer thereof;
or a pharmaceutically acceptable salt or solvate thereof, and wherein the purine group is subject to tautomeric rearrangements.
I-3. The micelle composition according to any one of the preceding items, wherein the TLR7 agonist is of formula (I):

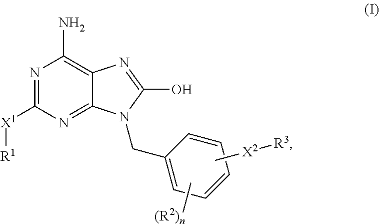

wherein X¹ is —O—, —S—, or —NR^{C};
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R^{C} is hydrogen, C₁₋₁₀alkyl, or substituted C₁₋₁₀alkyl; or R^{C} and R¹ taken together with the nitrogen to which they are attached form a heterocyclic ring or a substituted heterocyclic ring;
R² is absent;
n is 0;
X² is a bond or a linking group; and
R³ is a lipid;
or a tautomer thereof;
or a pharmaceutically acceptable salt or solvate thereof, and wherein the purine group is subject to tautomeric rearrangements.
I-4. The micelle composition according to any one of the preceding items, wherein X² is selected from the group consisting of: a bond, —O—, —C(O)— (carbonyl), (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₅-C₁₀)aryl (aroyl), substituted —C(O)—(C₅-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl); and
   each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
   and wherein
   X¹ is —O—, —S—, or —NR^{C};
   R¹ is hydrogen, (C₁-C₆)alkyl, or substituted (C₁-C₆)alkyl;
   R^{C} is hydrogen, C₁₋₆alkyl, or substituted C₁₋₆alkyl; or R^{C} and R¹ taken together with the nitrogen to which they are attached form a heterocyclic ring or a substituted heterocyclic ring.
I-5. The micelle composition according to any one of the preceding items, wherein R³ is a lipid selected from the group consisting of: a phospholipid comprising one or two carboxylic esters; a gonane, such as cholesterol; a saccharolipid; and a glyceride.
I-6. The micelle composition according to any one of the preceding items, wherein R³ is a phospholipid comprising one or two carboxylic esters.
I-7. The micelle composition according to any one of the preceding items, wherein the diameter of the micelle is between 5 nm and 50 nm, such as between 6 and 46 nm, such as between 7 and 42 nm, such as between 8 and 38 nm, such as between 9 and 34 nm, such as between 10 and 34 nm, such as between 11 nm and 30 nm, such as between 12 nm and 26 nm.
I-8. The micelle composition according to any one of the preceding items, wherein the diameter of the micelle is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.
I-9. The micelle composition according to any one of the preceding items, wherein the amphiphilic micelle-forming agent is selected from the group consisting of: a poloxamer, a poloxamine, a PEG-polyester, a PEG-polyanhydride, a PEG-poly-amino acid, a phospholipid, a polysorbate, and a polyoxyethylene alkyl ether.
I-10. The micelle composition according to any one of the preceding items, wherein the PEG-polyester is selected from the group consisting of: a PEG-poly(lactic acid) (PEG-PLA), a PEG-poly(lactic-co-glycolic acid) (PLGA), and a PEG-poly(ε-caprolactone) (PCL).
I-11. The micelle composition according to any one of the preceding items, wherein the PEG-polyanhydride is a PEG-polysebacic anhydride (PSA).
I-12. The micelle composition according to any one of the preceding items, wherein the PEG-poly-amino acid is selected from the group consisting of: a PEG-poly(L-histidine), a PEG-poly(L-aspartic acid), a PEG-poly(L-asparagine), a PEG-poly(L-glutamic acid), a PEG-poly(L-glutamine), and a PEG-poly(L-lysine).
I-13. The micelle composition according to any one of the preceding items, wherein the amphiphilic micelle-forming agent is a phospholipid conjugated to polyethylene glycol (PEG).
I-14. The micelle composition according to any one of the preceding items, wherein the phospholipid conjugated to PEG is conjugated via a carbonyl group.
I-15. The micelle composition according to any one of the preceding items, wherein the size of PEG is between PEG350 and PEG5000, for example between PEG550 and PEG4000, for example between PEG750 and PEG3000, such as between PEG1000 and PEG3000, preferably the size of the PEG is PEG2000.
I-16. The micelle composition according to any one of the preceding items, wherein the phospholipid comprises one or more alkyl chains that are C8-C24 alkyl(s), such as C10-C22, such as C12-C20, preferably C14-C18, most preferred C16-C18 saturated alkyl chains or unsaturated alkyl chains.
I-17. The micelle composition according to any one of the preceding items, wherein the phospholipid comprises a phosphatidylethanolamine (PE), a phosphatidylcholine (PC), a phosphatidylserine (PS), a phosphatidylglycerol (PG), a phosphatidylinositol (PI), a phosphatidic acid (PA), a bisphosphatidyl glycerol (DPG), or a phosphatidyl alcohol.
I-18. The micelle composition according to any one of the preceding items, wherein the phosphatidylethanolamine is selected from the group consisting of 1,2-dioleoyl-phosphatidylethanolamine, 1,2-dipalmitoyl-phosphatidylethanolamine, 1,2-dimyristoyl-phosphatidylethanolamine, 1,2-distearoyl-phosphatidylethanolamine, 1-oleoyl-2-palmitoyl-phosphatidylethanolamine, 1-oleoyl-2-stearoyl-phosphatidylethanolamine, 1-palmitoyl-2-oleoyl-phosphatidylethanolamine, and 1-stearoyl-2-oleoyl-phosphatidylethanolamine.
I-19. The micelle composition according to any one of the preceding items, wherein the phospholipid conjugated to PEG is selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.
I-20. The micelle composition according to any one of the preceding items, wherein the phospholipid conjugated to PEG is: 1,2-distearoyl-sn-glycero-3-phosphoethanolamine (DSPE)-PEG.
I-21. The micelle composition according to any one of the preceding items, wherein the amphiphilic micelle-forming agent is DSPE-PEG2000.
I-22. The micelle composition according to any one of the preceding items, wherein the molar ratio between the amphiphilic micelle-forming agent and the TLR7 agonist is from 50:50 to 99.5:0.5, such as from 60:40 to 99:1, such as from 70:30 to 98:2, such as from 80:20 to 95:5, for example 95:5, 90:10, or 80:20.
I-23. The micelle composition according to any one of the preceding items, wherein the composition comprises between 1% and 25% molar concentration of TLR7 agonist, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.
I-24. The micelle composition according to any one of the preceding items, wherein the TLR7 agonist has a structure according to formula (IA):

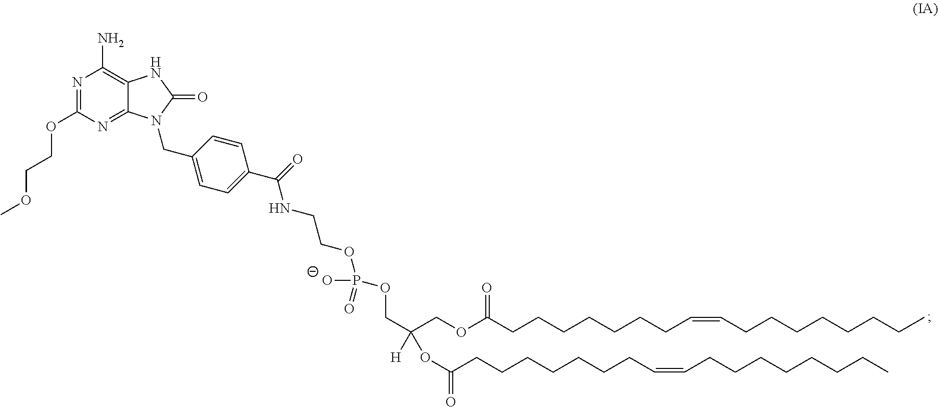 [SMILES: CCCCCCCC/C=C\CCCCCCCC(=O)OCC(COP(=O)([O-])OCCNC(=O)c1ccc(Cn2c(=O)[nH]c3c(N)nc(OCCOC)nc32)cc1)OC(=O)CCCCCCC/C=C\CCCCCCCC]

or a tautomer thereof;
   or a pharmaceutically acceptable salt or solvate thereof.
I-25. The micelle composition according to any one of the preceding items, wherein: the TLR7 agonist is of formula (IA);
   the amphiphilic micelle-forming agent is DSPE-PEG2000; and
   the ratio between the TLR7 agonist and amphiphilic micelle-forming agent is 95:5, 90:10, or 80:20.
I-26. The micelle composition according to any one of the preceding items, wherein the molar ratio between DSPE-PEG2000 and the TLR7 agonist of formula (IA) is 90:10 (MBS8).
I-27. The micelle composition according to any one of the preceding items, further comprising at least one further active ingredient.
I-28. The micelle composition according to any one of the preceding items, further comprising at least one antigen.
I-29. A pharmaceutical composition comprising the micelle composition according to any one of the preceding items.
I-30. A micelle composition or pharmaceutical composition according to any one of the preceding items, for use in the prevention, treatment or amelioration of a disease or disorder.
I-31. The micelle composition or pharmaceutical composition for use according to any one of the preceding items, wherein the disease or disorder is selected from the group consisting of: a cancer, an infectious disease, an inflammatory condition or disease, an autoimmune disease, and an allergy.
I-32. The micelle composition or pharmaceutical composition for use according to any one of the preceding items, wherein the disease or disorder is cancer, such as colon cancer.
I-33. A method for in vivo activation of immune cells in a subject, comprising administering the micelle composition or pharmaceutical composition according to any one of the preceding items to said subject in an amount sufficient to activate said immune cells.
I-34. A method for treatment of cancer in a patient in need of treatment, comprising administering the micelle composition as defined in any one of the preceding items to the patient.
I-35. The method according to any one of the preceding items, further comprising administering a chemotherapeutic agent to the patient.
I-36. The method according to any one of the preceding items, wherein the chemotherapeutic agent is selected from the group consisting of Doxorubicin, Doxil, Epirubicin, Cyclophosphamide, Bortezomib, and Oxaliplatin.
I-37. The method according to any one of the preceding items, further comprising administering an immune check point inhibitor to the patient.
I-38. The method according to any one of the preceding items, wherein the immune check point inhibitor is selected from the group consisting of: Atezolizumab, Avelumab, Durvalumab, Nivolumab, Tislelizumab, Pembrolizumab, and Ipilimumab.
I-39. The method according to any one of the preceding items, wherein the micelle composition is MBS8 and the immune check point inhibitor is selected from the group consisting of: Nivolumab and Pembrolizumab.
I-40. The method according to any one of the preceding items, further comprising administering a monoclonal antibody targeting CD20 to the patient.
I-41. The method according to any one of the preceding items, comprising administering MBS8 and a monoclonal antibody targeting CD20.
I-42. The method according to any one of the preceding items, further comprising administering a monoclonal antibody targeting the epidermal growth factor receptor (EGFR) to the patient.
I-43. The method according to any one of the preceding items, comprising administering MBS8 and a monoclonal antibody targeting the epidermal growth factor receptor (EGFR).
I-44. The method according to any one of the preceding items, further comprising administering a monoclonal antibody targeting the Human Epidermal Growth Factor Receptor 2 (HER2) to the patient.
I-45. The method according to any one of the preceding items, comprising administering MBS8 and a monoclonal antibody targeting the Human Epidermal Growth Factor Receptor 2 (HER2).
I-46. The method according to any one of the preceding items, further comprising administering a monoclonal antibody targeting CD38, such as Daratumumab or Isatuximab, to the patient.
I-47. The method according to any one of the preceding items, comprising administering MBS8 and a monoclonal antibody targeting CD38, such as Daratumumab or Isatuximab.
I-48. The method according to any one of the preceding items, further comprising administering a monoclonal antibody targeting CD47, such as Magrolimab, to the patient.
I-49. The method according to any one of the preceding items, comprising administering MBS8 and a monoclonal antibody targeting CD47, such as Magrolimab.
I-50. The method according to any one of the preceding items, further comprising radiotherapy.

### EXAMPLES

### Example 1: Micelle and Liposome Preparation

Micelles were made from 1,2-distearyl-sn-glycero-3-phosphoethanolamine-N-[methoxy(polyethylene glycol)-2000] (DSPE-PEG2000) obtained from Lipoid GmbH. Briefly, the lipid was dissolved in tert-butanol:water (9:1 ratio by volume) to a final concentration of 5-10 mM in glass vials, and put under magnet stirring and heating to 50 degrees Celsius until completely dissolved. The solvent was removed by freezing the vials in liquid nitrogen followed by overnight lyophilization. Micelles were prepared by dispersing the dried lipids in a buffer solution containing: 150 mM NaCl, 10 mM Phosphate (pH=7.4), exposing the vial to gentle vortexing to establish initial contact between lipids and solvent before exposing to ultrasonication for 30 minutes to ensure formation of micellar structures. The dispersion was vortexed once again, before exposing the dispersion to 30 min of further ultrasonication. The micelles were stored at 4 degrees Celsius before use and/or characterization.

Unilamellar fully hydrated liposomes were made from mixtures of 1-palmitoyl-2-oleoyl-sn-glycero-3-phosphocholine (POPC), 1-palmitoyl-2-oleoyl-sn-glycero-3-phosphoglycerol (POPG), 1,2-dioleoyl-3-trimethylammonium-propane (DOTAP), Cholesterol (Chol) and 1,2-dioleoyl-sn-glycero-3-phosphoethanolamine-N-[methoxy(polyethylene glycol)-2000] (DOPE-PEG2000) and 1v270 (C57H93N6O12P, Mw=1085.4, (2-(4-((6-amino-2-(2-methoxyethoxy)-8-oxo-7H-purin-9(8H)-yl)methyl)benzamido)ethyl 2,3-bis(oleoyloxy)propyl phosphate). The molar ratios of each lipid in the liposomes were MBS1: POPC:Chol:DOTAP:1V270:DOPE-mPEG2k (44.25:30:20:0.75:5), and MBS2: POPC:Chol:POPG:1V270:DSPE-PEG2k (44.25:30:20:0.75:5). All lipids were obtained from Avanti Polar lipids or Lipoid. Briefly, appropriate weighed amounts of POPC, POPG, Chol, DOTAP, 1V270 and DOPE-PEG2000 were dissolved in chloroform. The solvent was removed by a gentle stream of N₂ and the lipid films were dried overnight under low pressure to remove trace amounts of solvent. Multilamellar vesicles were prepared by dispersing the dried lipids in a buffer solution containing: 150 mM KCL, 10 mM HEPES (pH=7.5), 1 mM NaN₃, 30 μM CaCl₂ and 10 μM EDTA. The multilamellar vesicles were extruded ten times through two stacked 100 nm pore size polycarbonate filters as described by Mayer et al., Biochim. Biophys. Acta, 858, 161-168.

### Example 2: Micelle Preparation with Incorporation of a Toll-Like Receptor 7 (TLR7) Agonist

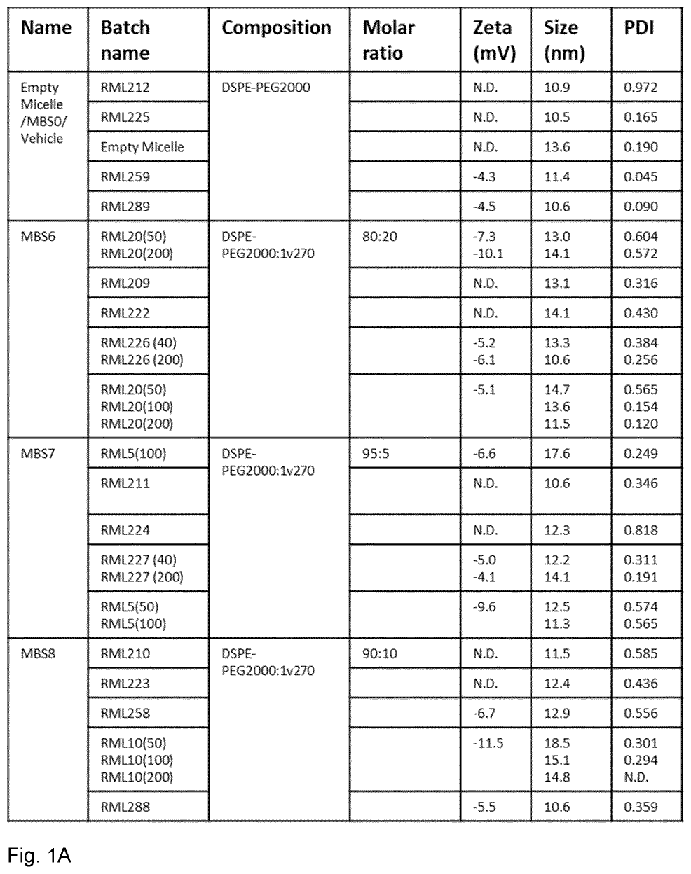

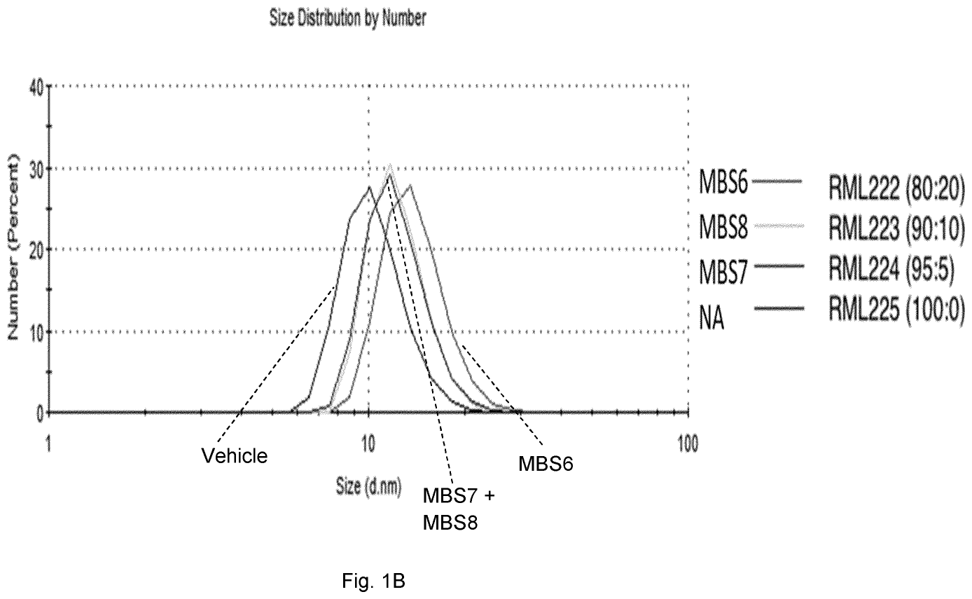

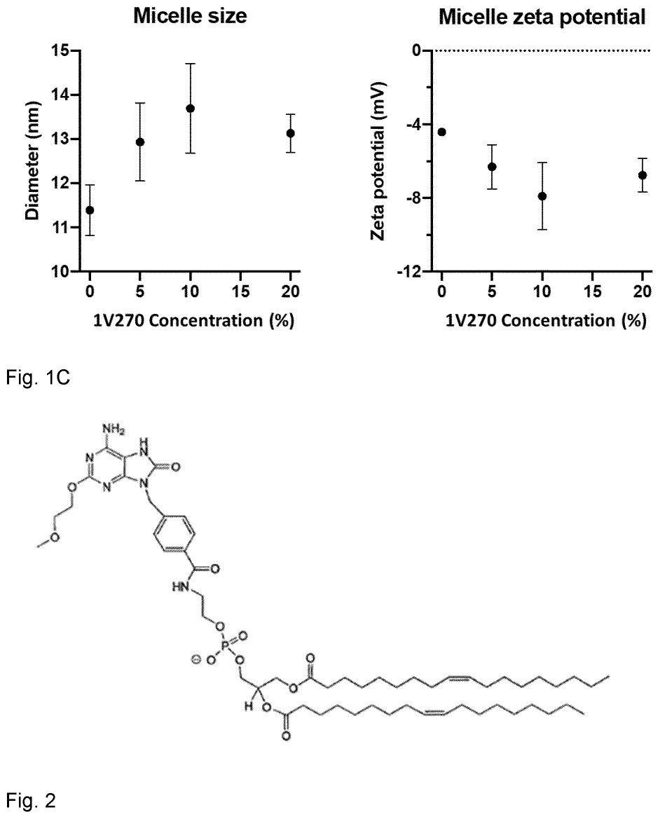

Micelles were made from 1,2-distearyl-sn-glycero-3-phosphoethanolamine-N-[methoxy(polyethylene glycol)-2000] (DSPE-PEG2000) obtained from Lipoid GmbH and the TLR7 agonist, 1v270 (C57H93N6O12P, Mw=1085.4, (2-(4-((6-amino-2-(2-methoxyethoxy)-8-oxo-7H-purin-9(8H)-yl)methyl)benzamido)ethyl 2,3-bis(oleoyloxy)propyl phosphate). The chemical structure of 1v270 is outlined in FIG. 2 Briefly, the lipid was dissolved in tert-butanol:water (9:1 ratio by volume) to a final concentration of 5-10 mM (DSPE-PEG2000) or 1-3 mM (1v270) in glass vials, and put under magnet stirring and heating to 50 degrees Celsius until completely dissolved. The two lipid dispersions were then mixed to the desired ratio (95:5 to 80:20 DSPE-PEG2000:1v270 molar ratio). The solvent was removed by freezing the vials in liquid nitrogen followed by overnight lyophilization. Micelles were prepared by dispersing the dried lipids in a buffer solution containing: 150 mM NaCl, 10 mM Phosphate (pH=7.4), exposing the vial to gentle vortexing to establish initial contact between lipids and solvent before exposing to ultrasonication for 30 minutes to ensure formation of micellar structures. The dispersion was vortexed once again, before exposing the dispersion to 30 min of further ultrasonication. The micelles were stored at 4 degrees Celsius before use and/or characterization. The batch names, molar ratios, lipid composition, zeta potentials (mV), size (nm) and polydispersity index (PDI) are outlined in FIG. 1.

### Example 3: Characterization of Micelle Size and Surface Charge Dependent on Composition

Micelles as outlined in FIG. 1 were prepared with the attempt to make a stable formulation of the TLR7 agonist 1V270 in aqueous solvents, thus allowing for injections in saline buffer. The micelles were prepared as described in examples 1+2, and their size (diameter) measured in nanometer (nm) by dynamic light scattering in a buffer consisting of 5% (w/w) glucose, 10 mM HEPES, 1 mM CaCl₂) in MilliQ water, pH 7.4. The micelles without 1V270 had a mean size of 11.5 nm (according to particle distribution by number), while micelles containing 1V270 had mean sizes ranging from 11.5 nm to 13-14 nm (according to distribution by particle number) when increasing 1V270 content from 5% to 20% (FIGS. 1B and 1C). The zeta potential of the empty micelles was measured in glucose buffer w. CaCl₂ to be around −4 mV, but becoming more negative when incorporating the anionic 1V270 compound into the micelles, thus decreasing to −8 mV for micelles with 10% 1V270 (FIG. 1C). Both size and zeta potential were measured on a Zetasizer from Malvern Instruments.

### Example 4: Antitumor Activity of Micelles and Liposomes Containing 1V270 in the CT26 Model

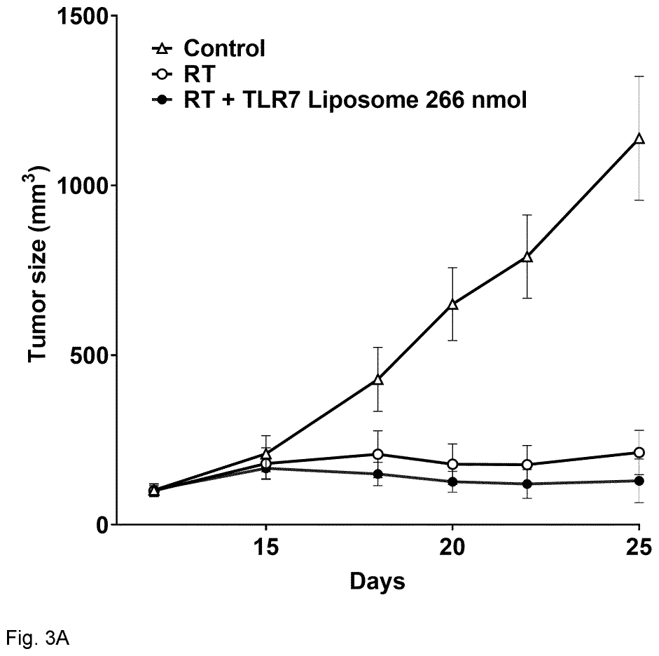

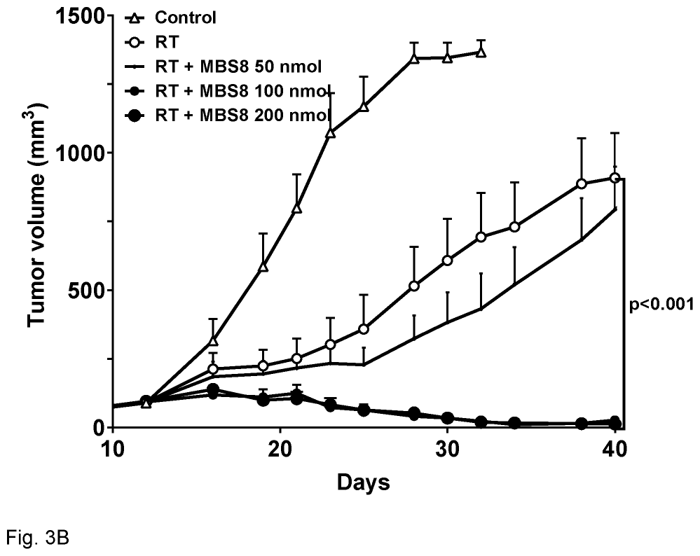

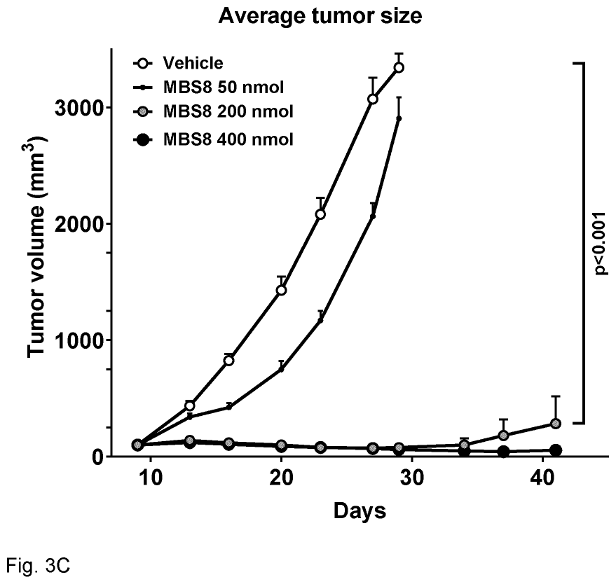

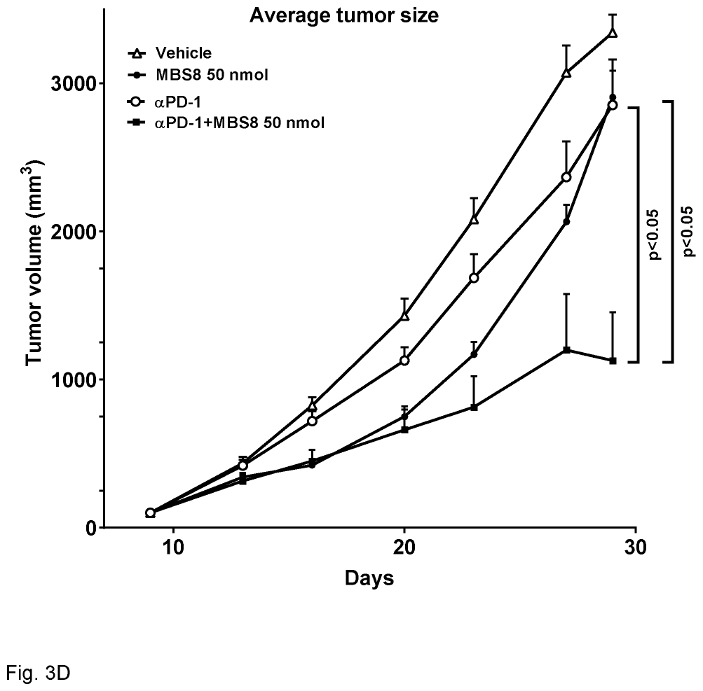

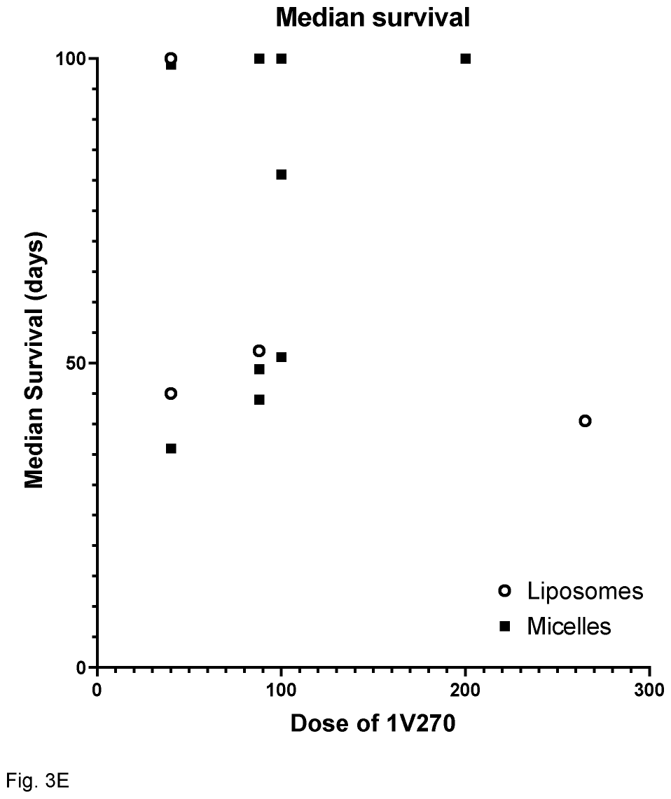

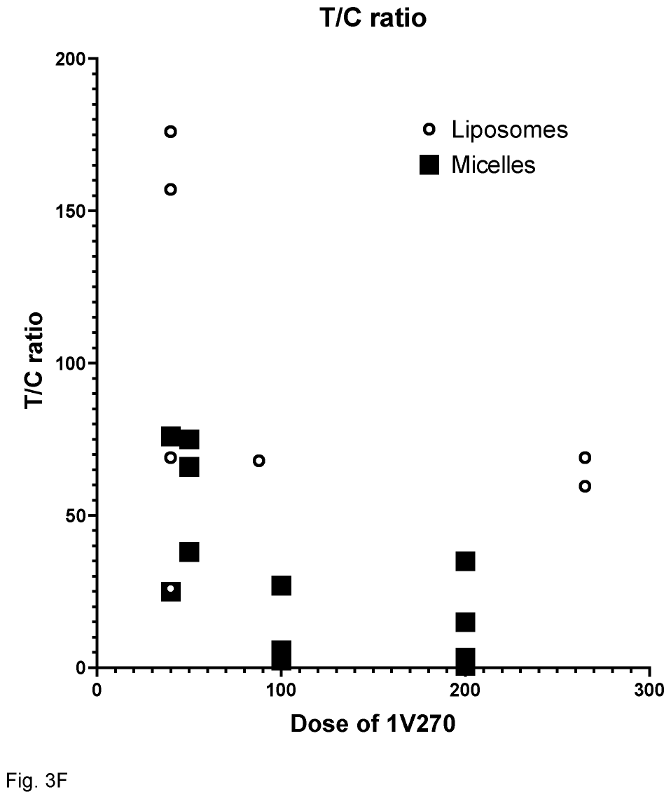

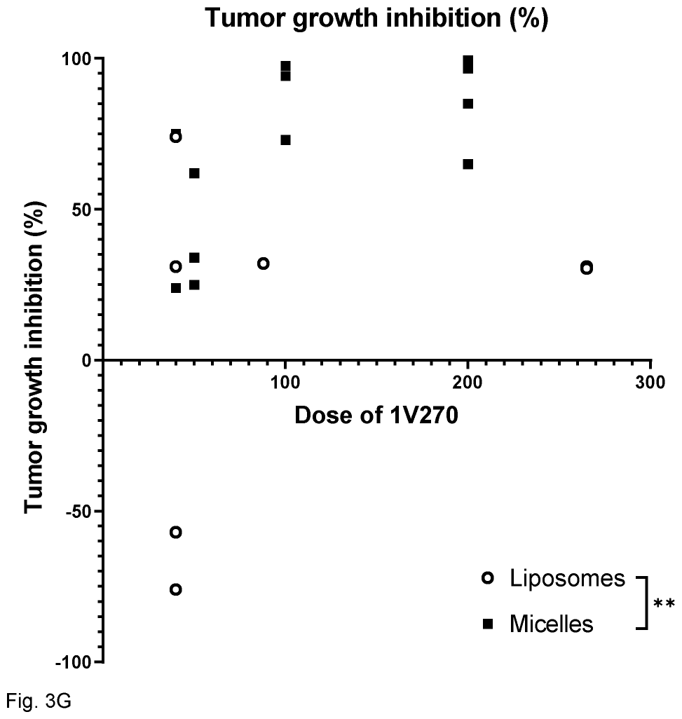

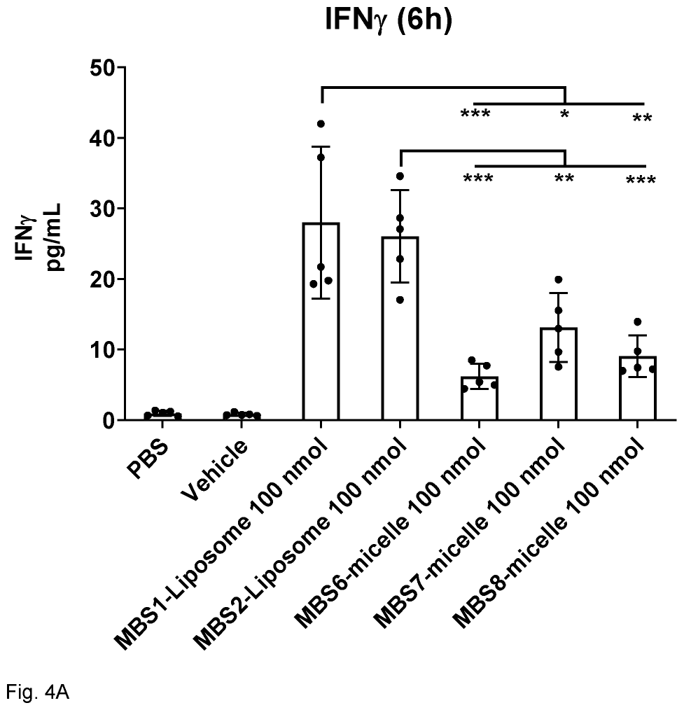

Tumor studies were conducted in the CT26 model (Adlard et al., Int J Cancer, 135, 820-829, 2014): Briefly the CT26 model is a colon cancer model established as a subcutaneous model in Balb/C mice which is frequently used as an immunocompetent tumor model to test cancer drugs and immunotherapeutic drugs. We used this model with control groups, radiotherapy treatment group from day 10 and daily for 5 days with 2 Gy. Liposomes or micelles containing 1V270 were injected IV at first day of radiotherapy and every four days after for five doses (FIGS. 3A and 3B). Liposomes containing 5% 1V270 were injected to reach a total dose of 266 nmol 1V270 per mouse (FIG. 3A). Micelles were injected at three doses of 50, 100 and 200 nmol 1V270 per mouse, and showed a significant tumor growth inhibition compared to radiotherapy treatment alone. Moreover, the lowest dose at 50 nmol showed 2/10 mice in complete remission, the 100 nmol dose showed 4/9 mice in complete remission, and the 200 nmol dose showed 8/8 mice in complete remission. In addition, nearly all mice in complete remission were able to resist a re-challenge with the same tumor cells after 100 days without tumor regrowth, demonstrating mice had generated an anti-tumor immune memory response. In monotherapy the MBS8 micelles were also very potent in showing anti-.tumor activity (FIG. 3C), where doses of MBS8 at 50, 200 and 400 nmol were injected intravenously in mice at day 9 and for additionally 4 times with 4 days interval. The two highest doses showed tumor growth suppression for all 10 mice in the groups lasting until 2 weeks after last dose (day 34). At day 41 there were three mice in complete remission in both high dose groups. The MBS8 micelles at 50 nmol/mouse/dose showed a synergy when combined with anti-PD1-treatment. This combo treatment showed a significantly better anti-tumor activity (p<0.05) than both anti-PD1 alone and MBS8 alone (FIG. 3D). anti-PD1 was dosed for 6 times IP at days 11, 15, 19, 23 and 27.

### Example 5: Antitumor Activity of Micelles Compared to Liposomes Containing a TLR7 Agonist in Combination with Radiotherapy in the CT26 Model

Multiple tumor studies were conducted with groups of mice treated with radiotherapy and 7 groups of liposome treated mice in the range from 40 to 266 nmol 1V270 injected per mouse, and 13 groups of mice treated with micelles from 50-200 nmol 1V270 injected per mouse in combination with radiotherapy. The number of mice per group was 7-9, and median tumor size was compared between groups treated with radiotherapy alone, and radiotherapy with either liposomes or micelles. The median survival time for each group of micelle or liposome treated mice is seen in FIG. 3E. 4 micelle treated groups showing a median survival above 100 days, indicating that more than half of the mice showed a complete tumor remission (the survival of the median observed mouse showed complete tumor remission) This was not observed for any liposome+RT treated groups. Tumor growth for liposome+RT and micelle+RT treated groups compared to groups treated with RT alone, (Termed T/C ratio) and calculated at the time when the RT alone treated group were sacrificed, showed a T/C ratio below 50% in 10 groups out 13 groups for micelles, whereas only 1 out of 7 liposome treated groups showed a T/C ratio below 50%. Using a Wilcoxon rank sum test the difference was significant at p<0.01 level (FIG. 3F). The T/C value can be translated into % tumor growth inhibition by 100% subtracted by the T/C-value (FIG. 3G), where values approaching 100% means a very strong inhibition of tumor growth. 10 out of 13 micelle treated groups showed a tumor growth inhibition above 60%, whereas only 1 of 7 liposome treated groups showed a tumor growth inhibition above 50%.

### Conclusion

In conclusion, micelles comprising 1V270 showed a significantly better tumor growth inhibition than the corresponding liposomes comprising 1V270 (p<0.01, Wilcoxon rank sum test).

### Example 6: Micelles Show Safer Cytokine Profile with Reduced Risk of Cytokine Release Syndrome (CRS)

The micelles MBS6, MBS7 and MBS8 containing the TLR7 agonist, 1V270 were compared to empty micelles (MBS0=vehicle), PBS and two different liposomes containing 1V270 by injection IV in Balb/C mice at a dose of 100 nmol 1V270 for all samples except PBS and vehicle. Plasma samples were taken at 2 and 6 h post injection and cytokines relevant for anti-tumor immune response and toxicity measured by multiplex or ELISA analyses.

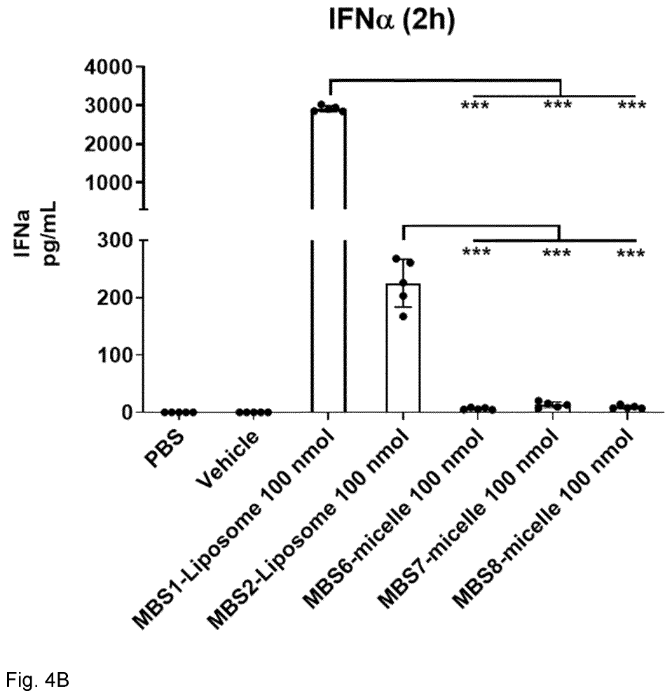

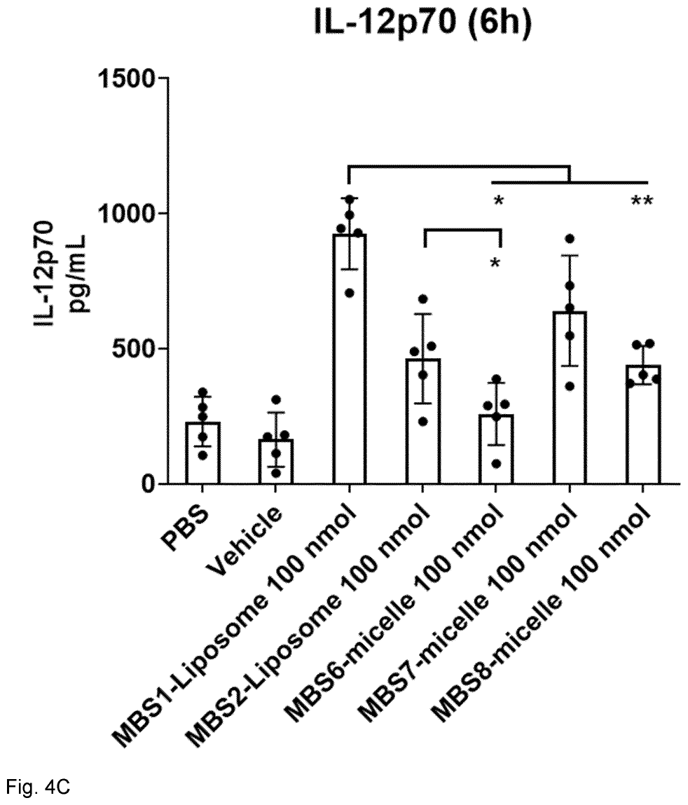

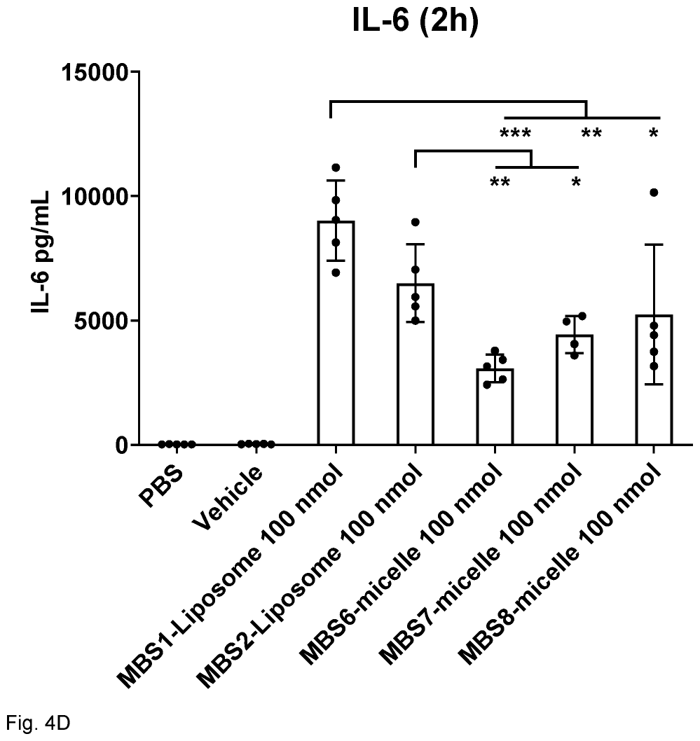

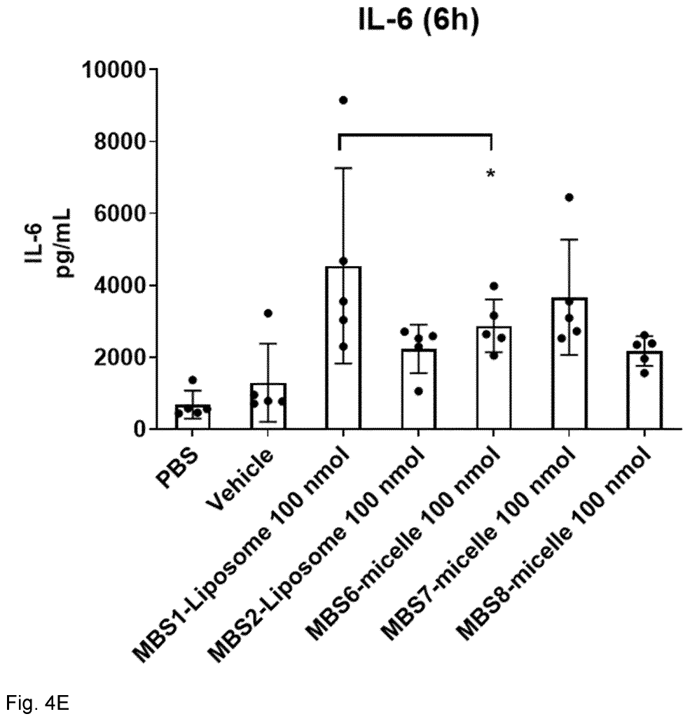

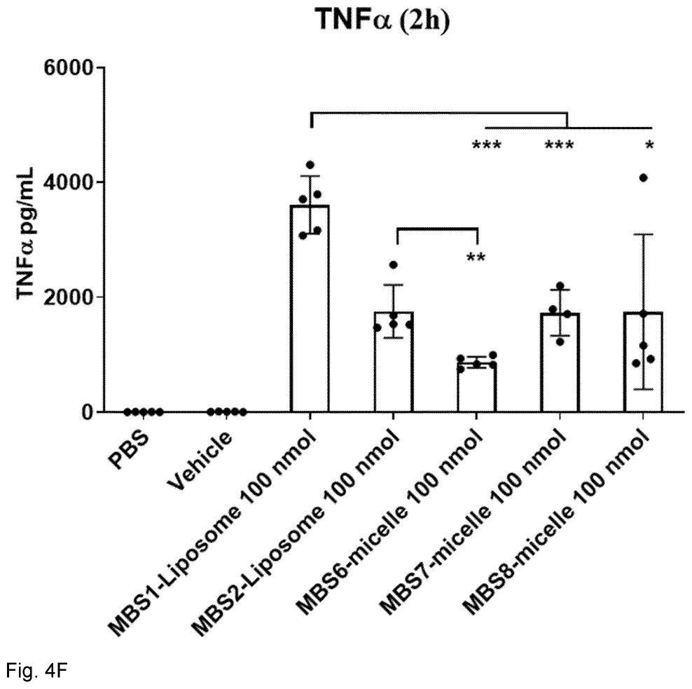

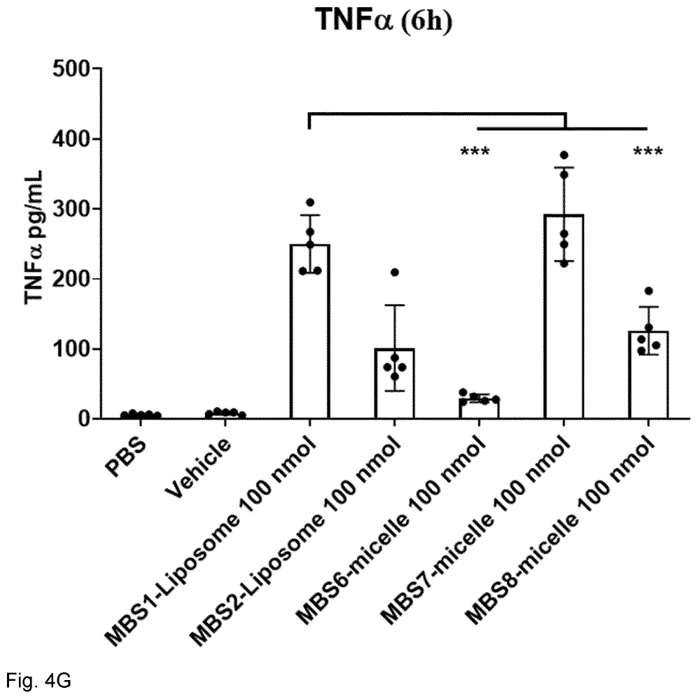

Both interferon gamma (FIG. 4A) and interferon alfa (FIG. 4B) were reduced for all three micelle formulations, MBS6-8, compared to the two liposome formulations at either 2 or 6 h post treatment, supporting a reduced risk of cytokine storm for the micelle formulations. IL-12p70 which is important for eliciting a type 1 cytotoxic T-cell response showed a slight but significant reduction for MBS6 and MBS8 treatment compared to MBS1 liposomes, but still a cytokine level expected to elicit an anti-tumor response (FIG. 4C). IL-6 which is a critical cytokine for initiation of CRS show reduced IL-6 level in plasma from mice treated with MBS6-8, compared to liposomes MBS1 and MBS2 at the 2 h time point. After 6 h, all mice showed lower IL-6 levels than after 2 h, with similar to or slightly lower IL-6 levels than the liposomes, demonstrating that the micelles show an overall reduction in IL-6 levels and not only a delay. TNF alpha which is also related to initiation of the CRS syndrome was also measured, and showed an overall cytokine reduction for micelles in particular compared to MBS1, and most pronounced for MBS6 and MBS8. Cytokines IL-1b and chemokine GRO were measured, but without showing significantly different cytokine levels.

### Conclusion

In conclusion, administration of micelles MBS6-8 in mice resulted in a safer cytokine profile with reduced risk of Cytokine Release Syndrome (CRS) compared to the corresponding liposomes MBS1 and MBS2 comprising 1V270.

### Example 7: Micelles Shows a Better Toxicology Profile Compared to Liposomes Containing 1V270

Multiple studies with liposomes of different charge and content of 1V270 ranging from 0.75-5% content of 1V270 and at doses of 13-266 nmol/mouse/dose were injected per mouse in groups of 7-9 mice/group. At the first two injections at day 0 and 4, the mice tolerated the liposomes well without any signs of adverse events or toxicity. However, at the third injection at day 8, mice showed adverse events and toxicity in 81% of the studies conducted. The toxicity was associated with lack of movement, piloerection, weight drop loss and general poor well-being for a transient period starting 10-15 minutes after dosing, and prolonged for 30-40 minutes. This observation was seen in 30 of 37 studies with Liposomes across all dose ranges.

In contrast, micelles did not show this toxicity in any studies, currently in 30 of 30 studies, indicating that micelle formulations of 1V270 show a better toxicity profile than liposome formulations of 1V270.

### Conclusion

In conclusion, administration of micelles MBS6-8 in mice resulted in better toxicology profile compared to the corresponding liposomes comprising 1V270.

### Example 8: Micelles Induce Lower IgM and IgG Anti-PEG Antibodies when Injected into Mice

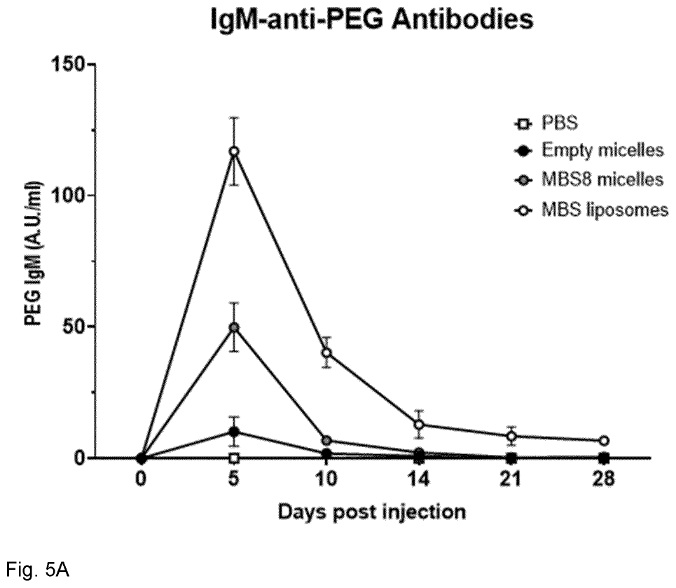

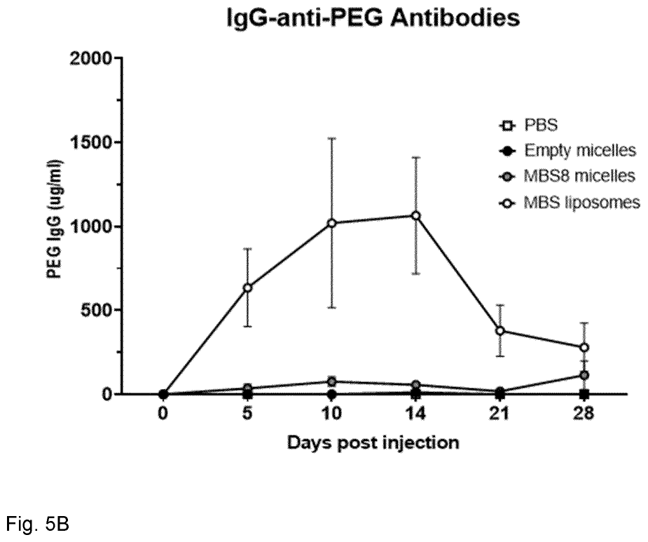

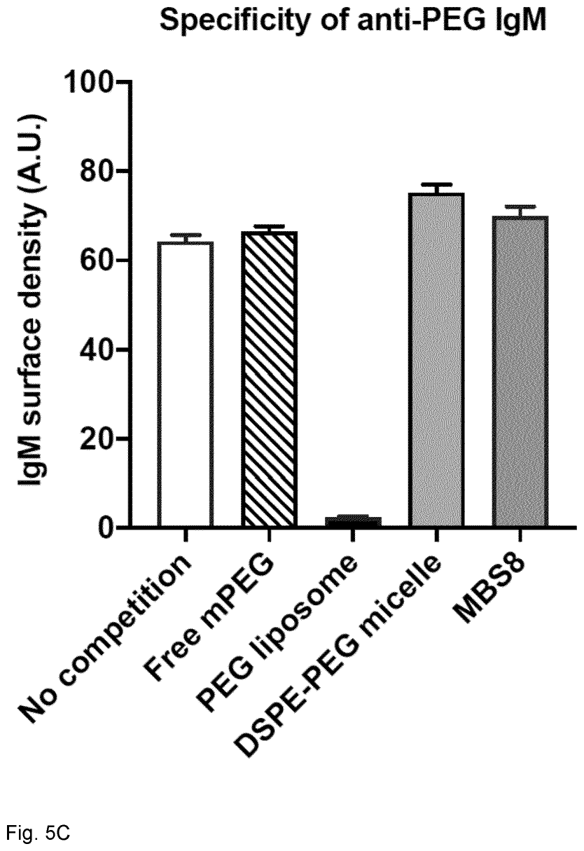

To explore the toxicity associated with liposomes containing 1V270 but not micelles containing 1V270, liposomes and micelles containing 1V270 were injected once by IV injection into mice, and blood samples drawn during a 28-day period. Micelles without 1V270 induced low levels of anti-PEG IgM antibodies in the mouse plasma when measured by an ELISA kit against anti-PEG IgM antibodies (FIG. 5A, empty micelles). MBS8 micelles induced anti PEG IgM molecules in plasma at day 5, but at levels lower than half of the amounts induced by liposomes containing 1V270 (FIG. 5A, MBS8 vs MBS liposomes). For anti PEG IgG antibodies which are normally more specific and present for longer times in mammals when induced, the induction was very high for liposomes 1-2 weeks after dosing and then dropped but was still present throughout the study (FIG. 5B). MBS8 micelles induced much lower IgG anti PEG antibodies (FIG. 5B, approximately 17 fold lower), than liposomes containing 1V270. This observation indicates that micelles induce lower levels of IgM and IgG anti PEG responses and thus reduce immune related toxicities in mice and potentially in other mammals, which is important in order to reduce toxicities in patients treated with micelles and liposomes with immune potentiating reagents.

FIG. 5C show specificity of anti-PEG antibodies generated against MBS8.

Healthy mice were treated with a single dose of MBS8 micelles at a total dose of 200 nmol 1V270. Blood was drawn at day 5 post injection, and plasma acquired. Next, the plasma was incubated with either PBS, PEGylated liposomes, free mPEG2000 chains, DSPE-PEG micelles without 1V270 or MBS8 micelles. The PEG concentration was 1 μM in all pre-incubations. The plasma was then added to microscopy wells containing immobilized fluorescently labeled PEGylated liposomes and incubated for 10 minutes. The microscopy well was washed, and fluorescent secondary antibodies against murine IgM added. The liposomes were imaged with confocal microscopy to determine surface density of IgM on the single liposomes.

When plasma had been pre-incubated with PBS (no competition) the surface density was approx. 60 A.U. Similar IgM binding was measured when plasma had been pre-incubated with free mPEG, DSPE-PEG micelles or MBS8 micelles, and showed a high IgM surface density (AU in the 60-70 range) indicating no competition for binding the anti-PEG IgM present in plasma. When plasma was pre-incubated with PEG-liposomes (positive control for competition), the anti-PEG IgM binding to immobilized PEG-liposomes was almost completely abolished, indicating strong competition for the binding. Thus, the anti-PEG IgM detected with ELISA in FIG. 5A induced by the MBS8 micelles are only able to recognize PEG chains attached to planar or liposomal surfaces, but not the PEG micelles (MBS8 included) or free PEG chains.

### Conclusion

The anti-PEG IgM generated against MBS8 micelles after injection into mice is not able to recognize and bind to MBS8 micelles or DSPE-PEG micelles in general, supporting that a potential generation of anti-PEG IgM antibodies in patients is unlikely to bind to MBS8 upon multiple injections.

### Example 9: Treatment of a Mammal with Cancer Using Immunostimulatory 1v270 Micelles (Non-Antigen Specific)

To obtain an immune stimulatory micelle suitable for cancer treatment, a micelle can be generated by mixing 1v270:DOPE-PEG2k (10:90=MBS8). The compounds are mixed in organic solvent and dried to a lipid film. This film is hydrated in a buffer suitable for intravenous administration, e.g. containing saline. The micelles are administered intravenously to a cancer patient suffering from e.g. lung cancer, breast cancer, prostate cancer, HNC, leukemia, lymphoma or melanoma with e.g. a one-two week interval. Combinations with clinically approved treatments is likely enhancing the anti-tumor effect. In particular in combination with immune checkpoint inhibitors, radiotherapy to boost the abscopal effect in e.g. lung cancer patients, to combine with mAb therapy like Rituximab and Trastuzumab to boost the Antibody Dependent Cell Cytotoxicity (ADCC), to enhance responses towards immunogenic cell death induced by certain chemotherapy like doxorubicin, oxaliplatin, cyclophosphamide and mitoxantrone.

### Example 10: Treatment of a Mammal with Cancer Using Immune Stimulatory 1v270 Micelles (Antigen Specific)

To obtain an antigen specific immune response micelles are prepared as in example 9, but with addition of an antigen peptide comprising whole or parts of the antigen of interest linked to a lipid anchor like e.g. DOPE. The peptide antigen associated with a lipid anchor ensures sufficient micelle association as seen for a 25 amino acid peptide sequence from the MUC1 tumor antigen, where a palmitoylated lysine residue ensures sufficient liposome association of the antigen (Sangha and Butts, Clin Cancer Res 2007; 13, 15 supp, 2007, 4652-54s). The antigen may be e.g. a MAGE antigen for treatment of melanoma, PSA for treatment of prostate cancer, a neoantigen or a third antigen or a combination of antigens. The antigen together with 1v270 are administered to a cancer patient expected to express the loaded antigen in their tumors. The micelles are administered to the same patient for a number of times to boost an antigen specific response, preferably with 1-2 weeks interval.

### Example 11: Antitumor Activity of MBS8 Micelles in Combination with Radiotherapy

Mice bearing CT26 subcutaneous tumors were treated with radiotherapy (RT) and micelles containing 50, 100, or 200 nmol 1V270. Micelles were given intravenously every fourth day for a total of 5 treatments starting from day 12 after tumor inoculation. 2 Gy RT to the tumor-bearing flank was given every day for 5 consecutive days starting from day 12 after tumor inoculation. The number of mice per group was 8-10 and mice were rechallenged with CT26 on the opposite flank on day 101 after primary inoculation. Data on tumor growth curves are mean tumor size±SEM.

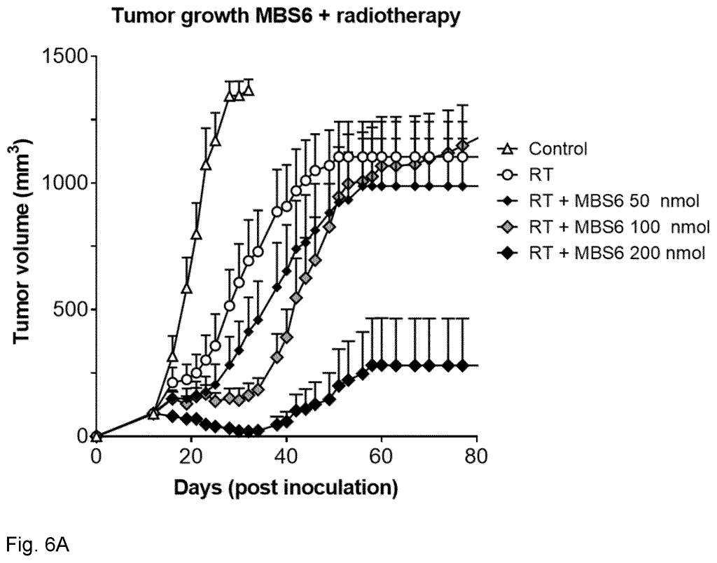

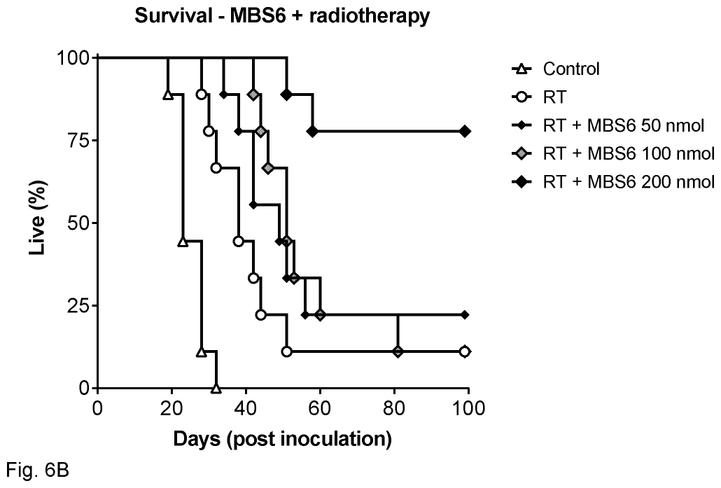

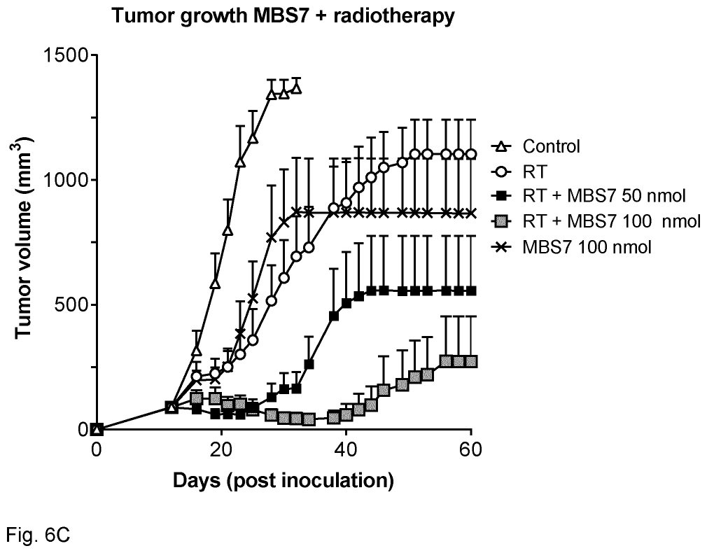

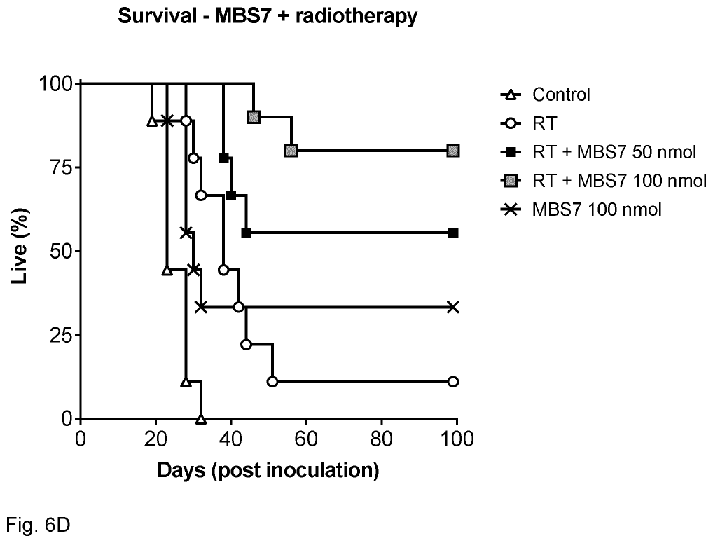

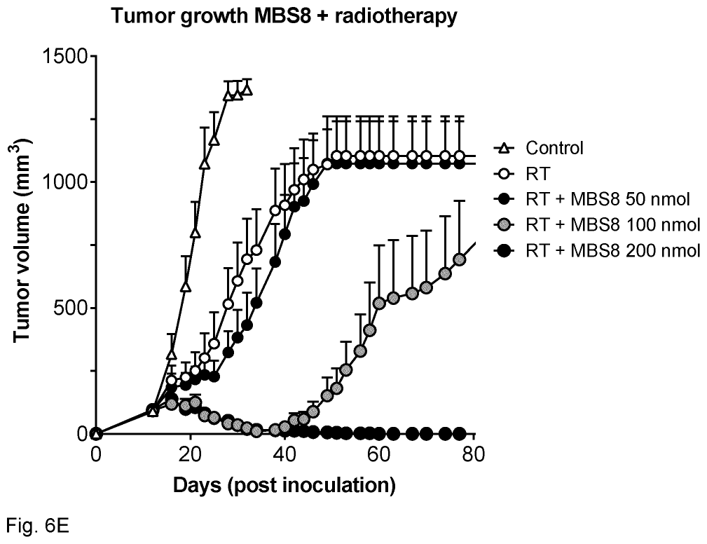

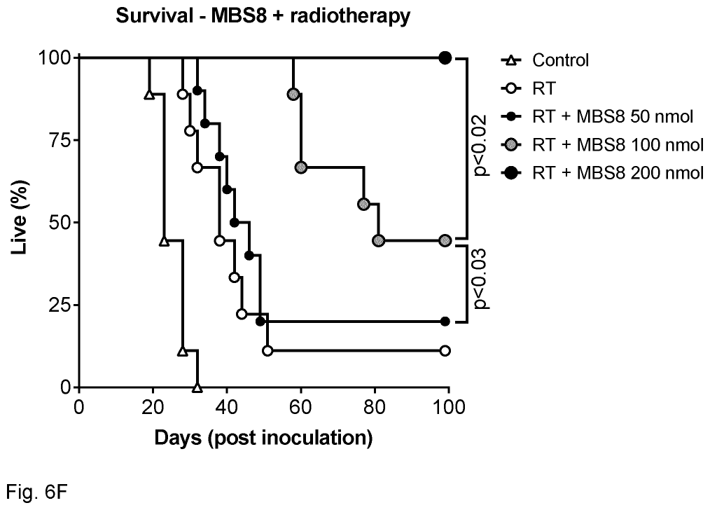

Combining 50 or 100 nmol MBS6 with RT provided only modest tumor control while 200 nmol MBS6 in combination with RT provided good tumor control (FIG. 6A). For RT alone, 1/9 mice were complete responders and 1/1 mice rejected rechallenge. For 50 nmol MBS6 combined with RT, 2/9 mice were complete responders and 2/2 rejected rechallenge. For 200 nmol MBS6 in combination with RT, 7/9 mice were complete responders and 7/9 rejected rechallenge (FIG. 6A-B).

Combining MBS7 with RT showed synergistic effect. For 50 nmol MBS7 combined with RT, 5/9 mice were complete responders and 5/5 rejected rechallenge. For 100 nmol MBS7 as monotherapy, 3/9 mice were complete responders and 3/3 rejected rechallenge. Combining 100 nmol MBS7 with RT provided 8/10 complete responders and 3/3 mice rejected rechallenge and provided significantly improved survival compared to monotherapy (p=0.02., Mantel-Cox test; FIG. 6C-D).

Combining 50 nmol MBS8 with RT provided 2/10 complete responders and 2/2 mice rejected rechallenge. For 100 nmol MBS8 in combination with RT, 4/9 mice were complete responders and 4/4 rejected rechallenge. For 200 nmol MBS8 in combination with RT, 8/8 mice were complete responders and 7/8 rejected rechallenge.

Furthermore, dose dependency could be observed for MBS8 in combination with RT (p<0.3, Mantel-Cox test; FIG. 6E-F).

### Conclusion

Micelles containing 1V270 in molar ratios of 80:20, 90:10 and 95:5 all show strong antitumor activity both in monotherapy and in combination with radiotherapy.

### Example 12: Antitumor Activity of MBS8 Micelles in Monotherapy

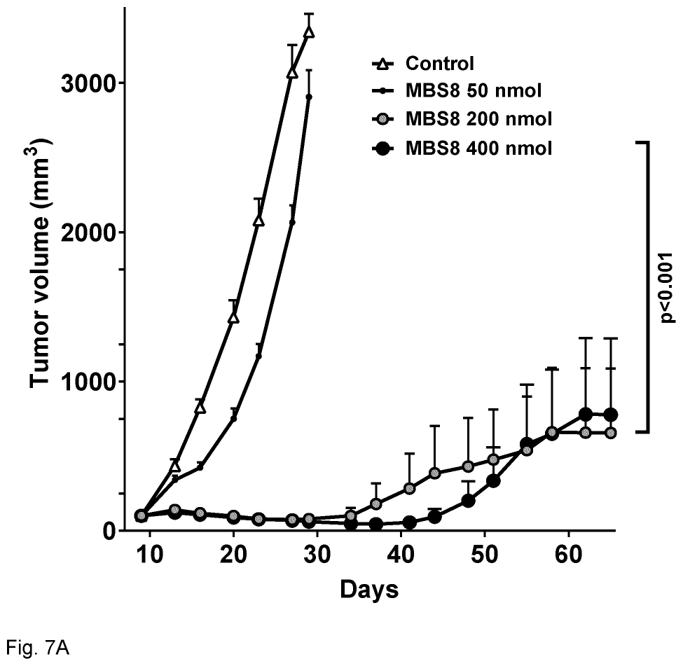

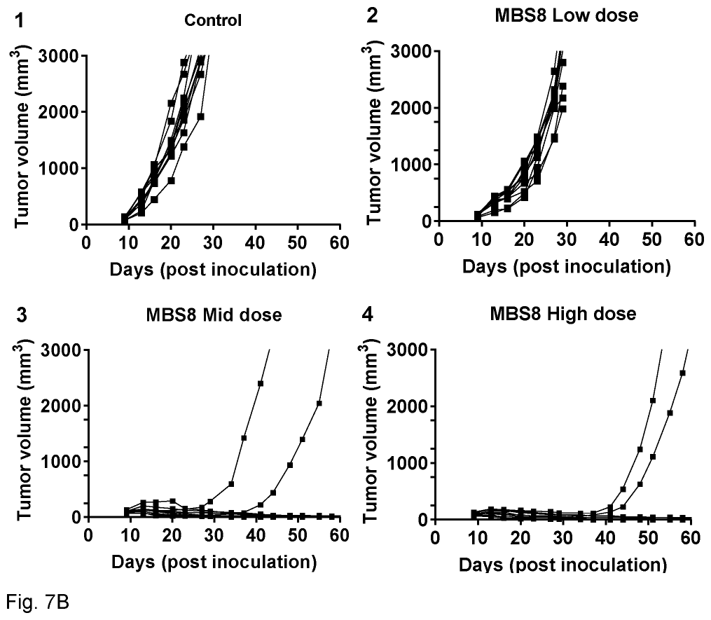

Efficacy of MBS8 micelles was studied in monotherapy in the CT26 syngenic subcutaneous colon cancer model. Randomisation of mice and treatment started when tumors reached an average volume of 100 mm³ (Day 0). Groups of 10 mice each were treated with PBS as vehicle control or three doses of MBS8 at 50, 200 or 400 nmol/mouse/injection by intravenous bolus (FIG. 7A). MBS8 was given q4d, total of 5 injections starting on day 9 (day 9, 13, 17, 21 and 25). Growth of tumors was measured twice a week. Mean tumor volumes of treated mice are shown in FIG. 7A, while tumor volumes of individual animals are shown in FIG. 7B (1-4). Statistical analysis using a Wilcoxon rank sum test revealed a significant potentiation of the efficacy of MBS8 at the mid and high dose compared to control (p<0.01). 8 mice out of 10 showed complete remission in the mid and high dose MBS8 treatment groups.

### Conclusion

MBS8 micelles comprising 1V270 in a 10 molar content show a significant antitumor activity at 200 and 400 nmol dose in the CT26 model, and are very potent in induction of complete remission.

### Example 13: Antitumor Activity of MBS8 Micelles in Combination with PD-1 Therapy

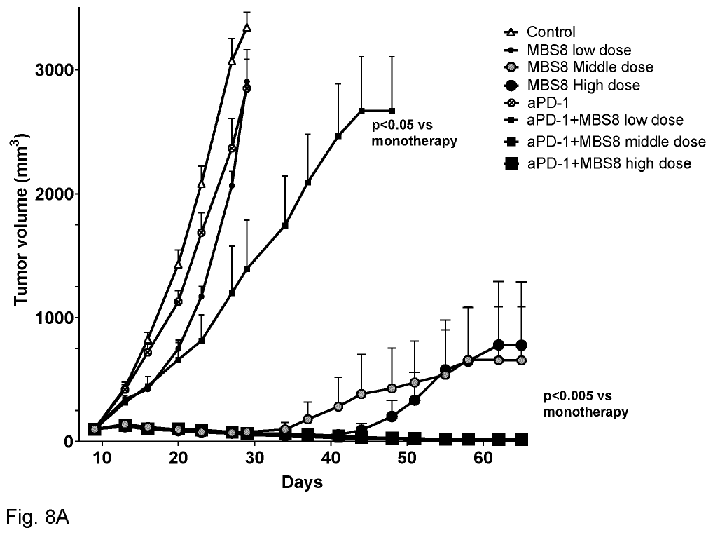

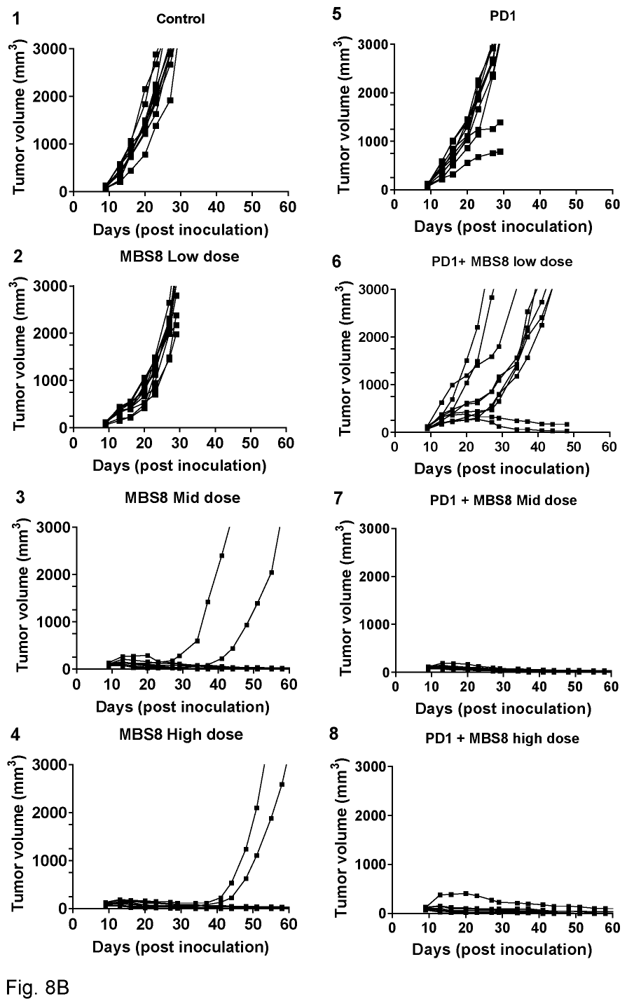

Efficacy of MBS8 micelles in combination with αPD-1 therapy was studied with a fixed dose of αPD-1 at 10 mg/kg injected IP and increasing dose of MBS8 at 50, 200 and 400 nmol/mouse/injection. The CT26 model was carried out and MBS8 treatment performed as described in Example 12. MBS8 treatment started day 9, and αPD-1 started day 11 and then twice weekly for three weeks (FIG. 8A). Low dose MBS8 in combination with αPD-1 showed a significant tumor growth delay (p<0.05) versus MBS8 low dose alone and αPD-1 alone (FIGS. 8A and 8B, graph 1-4). At the mid and high dose MBS8 the combination therapy did not initially show any difference since all 10 mice in each of the groups showed remission or very small tumors (FIG. 8A). However, at day 30-50 a few tumors started to grow in the MBS8 monotherapy groups with two escaping tumors (FIGS. 8A and 8B, graph 3-4). In the αPD-1 combo groups these escapers were not seen (FIG. 8B, graphs 7 and 8), and all tumors eventually disappeared or became very small (<30 mm3).

### Conclusion

MBS8 micelles comprising 1V270 are very potent in combination with αPD-1 leading to complete remission of at least 90% of treated mice carrying the CT26 tumor model.

### Example 14: Antitumor Activity of MBS8 Micelles in Combination with Chemotherapy (Doxorubicin and Doxil) in the CT26 Model

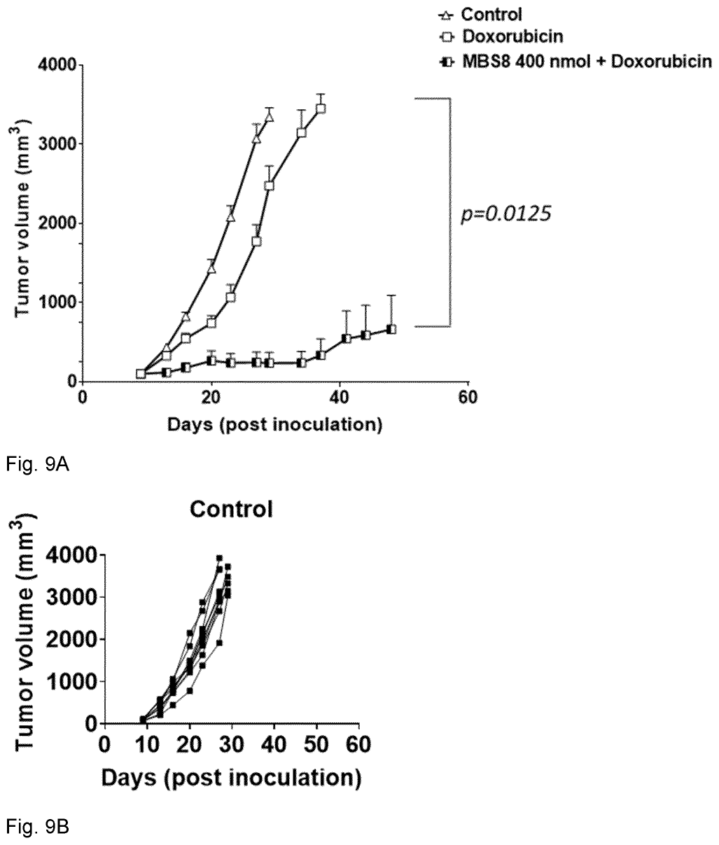

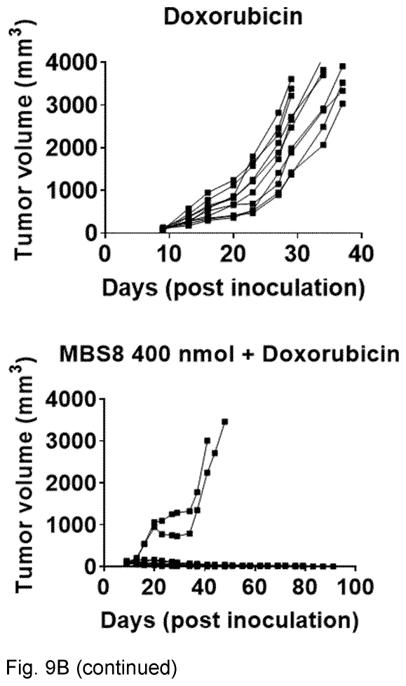

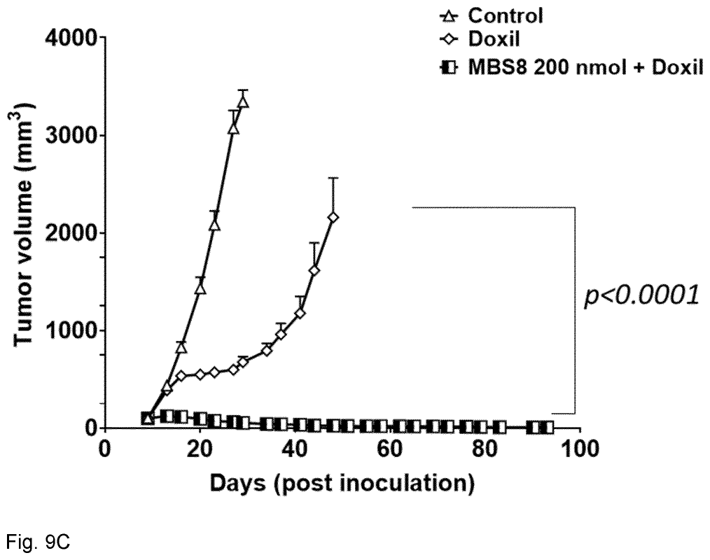

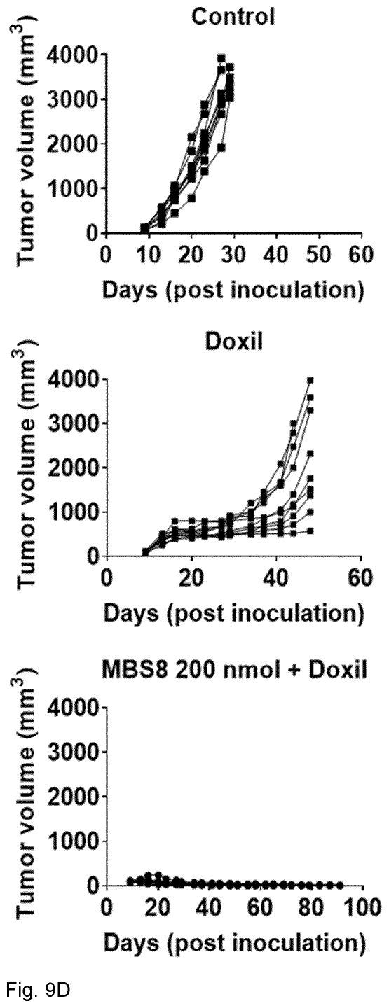

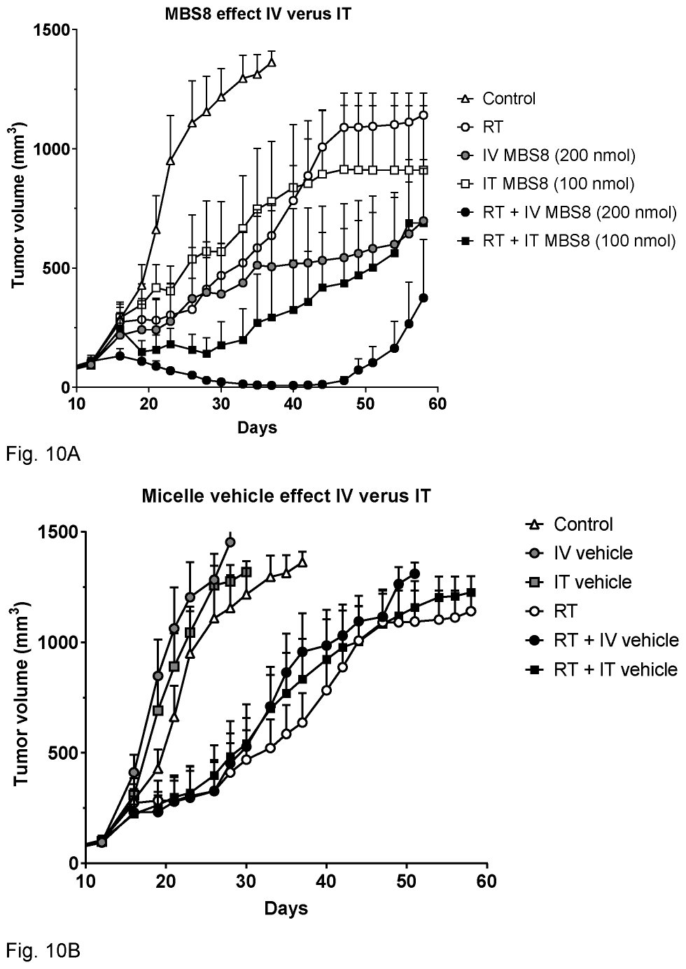

Efficacy of MBS8 micelles was studied in combination with standard of care chemotherapy, including doxorubicin and doxil, and compared with the efficacy of the monotherapy with these chemotherapeutic drugs in the CT26 syngenic subcutaneous colon cancer model. Randomisation of mice and treatment started when tumors reached an average volume of 100 mm³ (Day 0). Groups of 10 mice each were treated with PBS as vehicle control, doxorubicin (4 mg/kg) or combo of MBS8 (400 nmol/mouse) and doxorubicin (4 mg/kg) (FIG. 9A), or doxil (4 mg/kg) or combo of MBS8 (200 nmol/mouse) and doxil (4 mg/kg) (FIG. 9C). All drugs were administered by intravenous bolus. Doxorubicin or doxil were given q4d, total of 3 injections. MBS8 was given q4d, total of 5 injections. Doxorubicin or doxil were administered on Day 10, Day 14 and Day 18; MBS8 was administered on Day 10, 20, 24, 28 and 32. Growth of tumors was measured twice a week. Mean tumor volumes of doxorubicin and/or MBS8 treated animals are shown in FIG. 9A, while tumor volumes of individual animals are shown in FIG. 9B. Mean tumor volumes of doxil and/or MBS8 treated animals are shown in FIG. 9C, while tumor volumes of individual animals are shown in FIG. 9D. Statistical analysis using a Wilcoxon rank sum test revealed a significant potentiation of the efficacy of the chemotherapeutic drugs when combined with MBS8. All animals treated with MBS8/doxil combo showed a complete response and 8 out of 10 complete responders were in the group treated with MBS8/doxorubicin; no complete responders were found in the mono chemotherapy groups.

### Conclusion

Micelles comprising 1V270 in a 10 molar content significantly potentiate efficacy of doxorubicin and doxil and leads to complete responders.

### Example 15: Antitumor Activity of MBS8 Micelles Using Intravenous Versus Intratumoral Administration

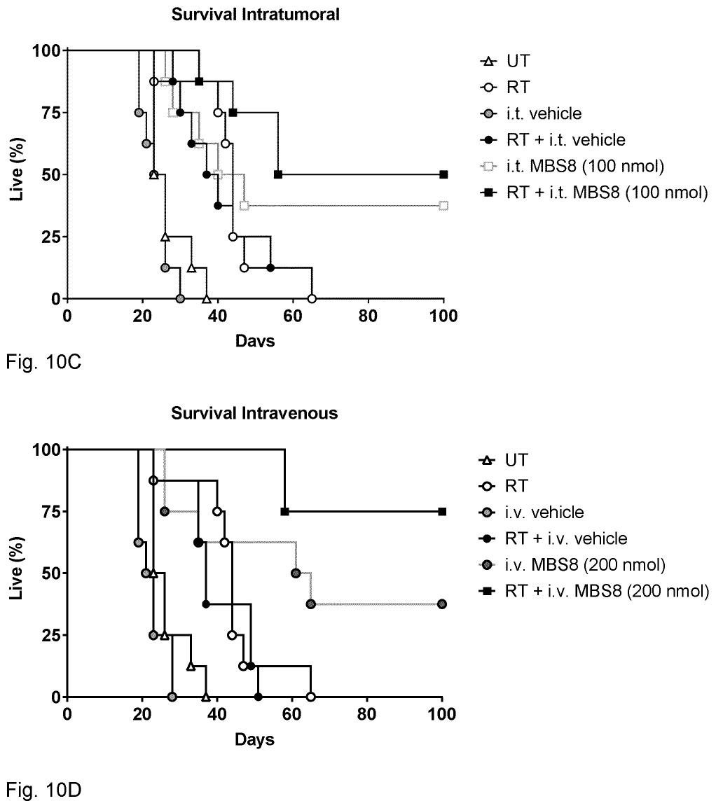

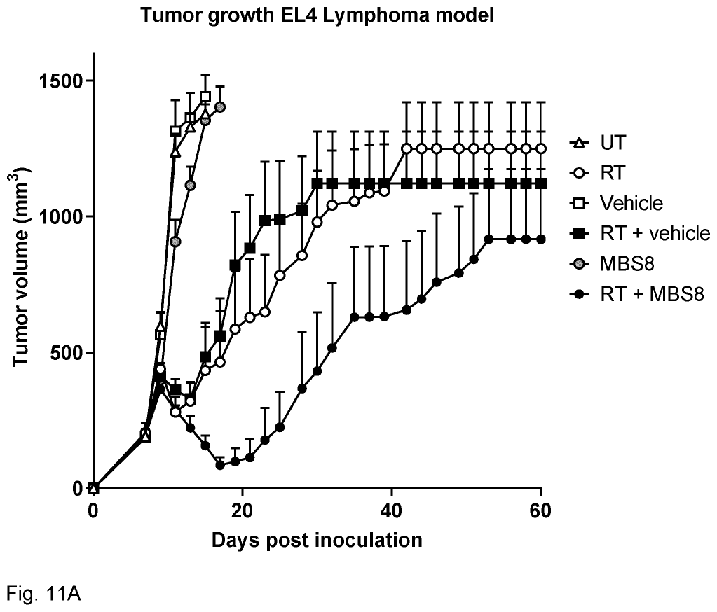

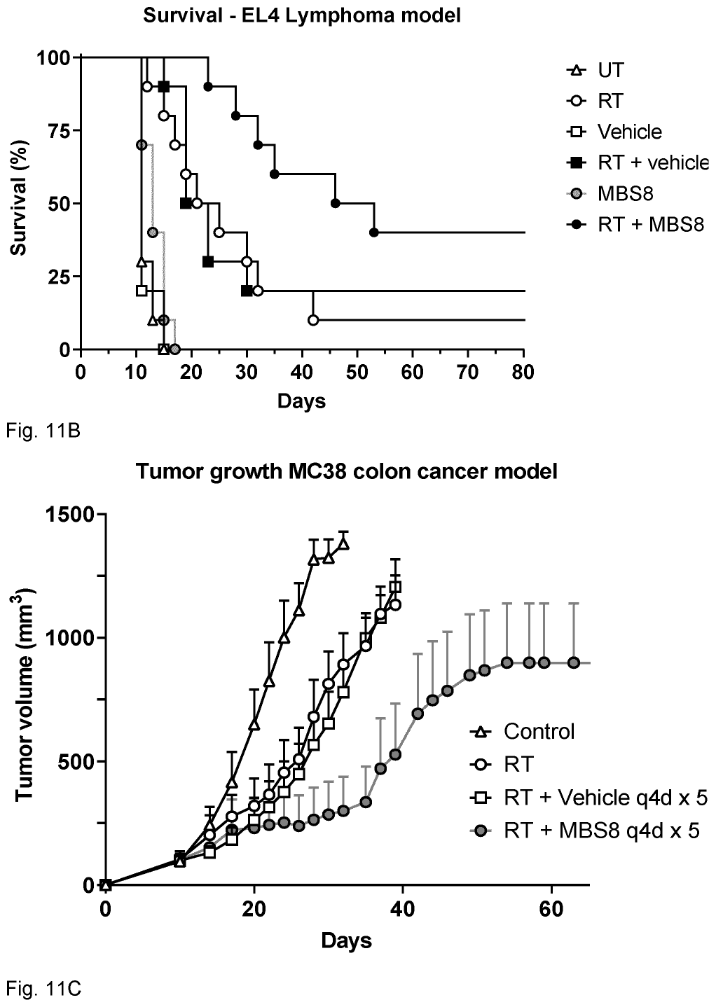

Mice bearing CT26 subcutaneous tumors were treated with radiotherapy (RT) and injection of MBS8 intravenously or intratumorally. MBS8 or lipid matched micelle vehicle without TLR7 agonist 1V270 was given every fourth day for a total of 5 treatments starting from day 12 after tumor inoculation. 2 Gy RT to the tumor-bearing flank was given every day for 5 consecutive days starting from day 12 after tumor inoculation. Intratumoral MBS8 injections were given as 100 nmol 1V270 due to volume limitation and intravenous MBS8 injections were given as 200 nmol 1V270 (FIG. 10A). The number of mice per group was 8 and mice in complete remission were rechallenged with CT26 on the opposite flank on day 103 after primary challenge. Data on tumor growth curves are mean tumor size±SEM. MBS8 provided good tumor control as monotherapy and in combination with radiotherapy regardless of administration route, but with the IV route slightly more potent than IT administration (FIG. 10A). Intravenous injection of MBS8 alone resulting in 3/8 complete responders of which 3/3 rejected rechallenge. Intravenous injection of MBS8 in combination with RT resulted in 6/8 complete responders of which 5/6 rejected rechallenge. Intratumoral injection of MBS8 alone resulted in 3/8 complete responders of which 3/3 rejected rechallenge. Intratumoral injection of MBS8 in combination with radiotherapy resulted in 4/8 complete responders of which 4/4 rejected rechallenge.

In contrast, vehicle injections did not improve tumor control (FIG. 10B). Radiotherapy alone or in combination with vehicle did not result in any complete responders. Survival of mice in the individual groups are shown for the intratumoral administration (FIG. 10C), and for the intravenous groups (FIG. 10D). Median survival days were for MBS8 treatment in monotherapy for IT administration 43.5 days versus 63 days for the IV administration. For MBS8 in combination with radiotherapy the median survival time was 56 days for the IT administration versus 100+ days for the IV administration, since 6 of 8 mice were in complete remission.

### Conclusion

MBS8 micelles containing 1V270 provided good tumor control when injected either intravenously or intratumorally and showed synergistic effect with radiotherapy regardless of administration route.

### Example 16: Antitumor Activity of MBS8 Micelles in Combination with Radiotherapy

Mice bearing MC38, EL4, or B16-F10 tumors were treated with radiotherapy (RT) and MBS8 intravenously. 200 nmol MBS8 or lipid matched vehicle was given at q4d for a total of 5 treatments. For EL4 bearing mice, treatment was started on day 7 after inoculation, micelles were given as q4d, and radiotherapy was given as 2 Gy to the tumor-bearing flank on 3 consecutive days with 9-10 mice per group. For MC38 bearing mice, treatment was started on day 10 after inoculation and radiotherapy was given as 2 Gy to the tumor-bearing flank on 5 consecutive days with 9 mice per group. MC38 bearing complete responders were rechallenged with MC38 on the opposite flank on day 80 after primary challenge.

MBS8 provided no tumor control in EL4 as monotherapy but in combination with RT resulted in 4/10 complete responders. RT alone only resulted in 1/9 complete responders and vehicle combined with RT resulted in 2/10 complete responders (FIG. 11A-B).

MBS8 provided moderate tumor control in MC38 bearing mice when combined with radiotherapy. RT combined with MBS8 given q4d resulted in 3/9 complete responders of which 2/3 rejected rechallenge. (FIG. 11C-D).

### Conclusion

In conclusion, MBS8 provides good tumor control in EL4 and MC38 when combined with RT.

### Example 17: Tolerability of MBS8 Micelles in Monkeys

Tolerability of MBS8 micelles was assessed in cynomolgus monkeys (Macaca fascicularis). MBS8 was administered into three naive male monkeys, body weight ˜3 kg, every 14 days with the following dose escalation scheme: 0.01 mg/kg→0.03 mg/kg→0.1 mg/kg→0.3 mg/kg→0.9 mg/kg→2.7 mg/kg. Administration of the drug was done by intravenous infusion of a total of 5 ml with the rate of 0.25 ml/min. During acclimation period, blood samples were taken at Day −14 and Day −7 as baseline controls and during treatment period at 2 h, 4 h, 8 h, 24 h, 72 h and Day 14. Hematology was analysed at 8 h, 24 h, 72 h and Day 14. Blood chemistry was analysed at Day 14 after each drug administration, i.e. immediately prior to the next dosing. C-reactive protein (CRP) was measured at 4 h, 8 h, 24 h, 72 h and Day 14 post-administration. Body temperature was monitored daily. Blood pressure was measured at 2 h, 4 h, 8 h after each drug administration and then daily until the next dose. Food consumption was checked daily and body weight—once a week.

All doses were well tolerated. A transient dose-dependent increase in the CRP level was detected 24 h post-infusion, which returned to baseline level within 3 days (FIG. 12A). There were no significant changes in body temperature throughout the study (FIG. 12B). Based on the blood chemistry, there were no signs of liver toxicity or changes in electrolyte profile.

### Conclusion

Intravenous infusion of MBS8 is well tolerated in cynomolgus monkey within the dose range of 0.01-2.7 mg/kg.

### Example 18: Antitumor Activity of MBS8 Micelles and αPD-1 in Monotherapy and Combination Therapy in Multiple Syngeneic Tumor Models

Efficacy of MBS8 as monotherapy or in combination with anti-PD1 monoclonal antibody (clone RMP1-14) was studies in a panel of 12 syngeneic mouse models (FIG. 13). Mice were inoculated s.c. with cancer cells; when average tumor size reached ˜100 mm³, the animals were randomised into 4 treatment groups of 10 animals each: Group 1, vehicle control (PBS); Group 2, anti-PD-1, 10 mg/kg; Group 3, MBS8, 300 microgram/mouse; Group 4, combination of MBS8 and anti-PD-1. Vehicle or MBS8 were administered by slow i.v. bolus, at a q4d schedule, 5 injections in total, starting at the day of randomization. Anti-PD-1 was administered i.p. starting two days after randomization, at a q4d schedule, 6 injections in total. Summary of results is shown in FIG. 13A. TGI %, tumor growth inhibition, was calculated as: TGI (%)=100×(1−T/C), where T and C are the mean tumor volumes of treated and control groups, respectively, on a day when the control group was terminated.

Three different patterns of response were observed:
(1) MBS8 was very potent in monotherapy and hence the benefit of combination with anti-PD-1 cannot be noted due to too strong MBS8 activity (FIG. 13B). The tumors falling into this category were: CT-26 (colon cancer), EMT-6 (breast cancer), A20 (B-cell lymphoma) and H22 (hepatoma).
(2) Both anti-PD-1 and MBS8 showed therapeutic activity in monotherapy. Combo therapy showed an additive benefit (FIG. 13C). This was observed for Hepa1-6 (hepatoma) and M38 (colon cancer).
(3) Anti-PD-1 was inactive in monotherapy while MBS8 monotherapy showed efficacy. Combination of the two drugs resulted in synergistic activity by turning PD-1 non-responsive mice into responsive (FIG. 13D). In this category were RM-1 (prostate cancer), Pan02 (Pancreatic cancer) and Renca (kidney cancer).

For the EMT-6 model, re-challenge of complete responders from Group 3 and 4, which were tumor-free for at least three weeks, was done with EMT-6 cells injected s.c. on the contralateral flank. The mice were followed for 29 days. All re-challenged animals showed complete rejection of re-challenged tumors (FIG. 13E). In CT26 model, mice which showed complete response to MBS8 or combination of MBS8 with anti-PD-1 therapy and who rejected re-challenged tumors were analysed for the presence of tumor-specific T-cells (FIG. 13F, Elispot graph). Splenocytes from untreated mice bearing CT26 tumors, from mice treated with either MBS8 or MBS8 and anti-PD-1 or naïve tumor-free mice were stimulated in vitro with an AH-1 tumor specific antigen peptide. IFNγ producing cells were quantified using ELISPOT. All mice which rejected re-challenged tumors demonstrated increased numbers of IFNγ positive cells in response to antigen stimulation thus confirming establishment of the immune memory response.

Conclusion: MBS8 showed therapeutic efficacy in monotherapy. In some tumors, which are weakly responsive to anti-PD-1, combination treatment with MBS8 had an additive effect. In models which were unresponsive to anti-PD-1 treatment, but responsive to MBS8, the latter sensitised tumors to anti-PD-1 and the drugs showed a strong synergistic effect. MBS8 in either monotherapy or combination treatment with anti-PD-1 led to establishment of the immune memory response.

### Example 19: Dose Schedule Optimization of MBS8 and Correlation to Anti-Tumor Activity in the CT26 Model

The CT26 colon cancer syngeneic mouse model was used to analyze the influence of dose schedule on the efficacy of MBS8 monotherapy. Mice with established tumors were treated with 200 nmol/mouse MBS8 at (1) single injection; (2) two injections at q7d; (3) three injections at q7d; (4) four injections at q7d and (5) five injections at q7d (FIG. 14). As control, vehicle (PBS) treatment was used as well as MBS8 treatment administered in five injections q4d, the schedule which showed good efficacy in previous studies.

Already single injection showed significant inhibition of tumor growth, while multiple weekly administration led to a complete eradication of tumors.

Conclusion: Only one single injection of MBS8 leads to a significant anti-tumor activity, while additional injections with either 4 or 7 days schedule leads to multiple mice in complete remission

### Example 20: Antitumor Activity of MBS8 Micelles in Comparison to R848 Monotherapy

Efficacy of MBS8 micelles and R848 was studied as monotherapy in the CT26 syngeneic subcutaneous colon cancer model. Randomisation of mice and treatment started when tumors reached an average volume of 85 mm³ (Day 11). Groups of 9 mice each were treated with vehicle control (MBS0); MBS8 at 100, 200 or 300 nmol/mouse/injection by intravenous bolus or with R848 at 200 nmol/mouse/injection. Treatments were given q4d, total of 5 injections starting on day 11 (day 11, 15, 19, 23 and 27). Growth of tumors was measured twice a week. Mean tumor volumes of treated mice are shown in FIG. 15A, while a survival curve is shown in FIG. 15B. Statistical analysis using a Wilcoxon rank sum test revealed a significant difference between all doses of MBS8 compared to vehicle (p<0.0001). Furthermore, MBS8 at 200 nmol was significantly more efficacious than 200 nmol R848 (p<0.05). Weight change to baseline is shown in FIG. 15C. For the MBS8 micelles weight loss change becomes less severe with more treatments whereas R848 induced the most severe weight loss change from day 25 to day 40.

### Conclusion

MBS8 micelles show a better anti-tumor effect than R848 when given in equimolar doses. Due to enhanced therapeutic activity and less severe weight loss, the MBS8 micelles have increased the therapeutic index compared to R848 of a TLR7 agonist.

### Example 21: Tumor Gene-Expression Analysis with Nanostring of MBS8 Treated CT26 Tumor Bearing Mice

The gene expression profile of CT26 tumors from mice treated with MBS8 as monotherapy was made to determine MBS8 treatment associated gene expression and presence of specific tumor cell types. Gene expression was evaluated with the Pan Cancer Immune Panel (Nanostring) performed on bulk RNA extracted from tumors. Mice were randomized on day 12 into groups of 10 and treated with MBS8 at 200 nmol/mouse/injection by intravenous bolus given in a q4d schedule for a total of 1 or 3 injections. Mice were sacrificed and the tumor snap frozen at days: day 0 (untreated), 1 day post 1^{st} injection, 2 days post 1^{st} injection, 4 days post 1^{st} injection, 14 days post 1^{st} injection, 2 days post 3^{rd} injection, 4 days post 3^{rd} injection, n=3-6 for all groups. RNA was extracted and 750 genes were analyzed with the Pan Cancer Immune Panel. A cell type analysis was performed with the advanced analysis module of the Nanostring software. Two-way ANOVA with multiple comparison correction was performed to compare the different time points to untreated. At the early time points (1, 2 and 4 days post 1^{st} injection) a significant increase in both neutrophils and dendritic cells is seen demonstrating activation of the innate immune system. At later time points (4 days after 1^{st} injection and later) T cells and especially CD8 T cells are increased in the tumor demonstrating activation of the adaptive immune system. Furthermore, macrophages are also upregulated at the later time points. The time point 14 days post 1^{st} injection have a cell type profile resembling UT, indicating that one injection in this setup is not enough to induce lasting immune response.

### Conclusion

The gene expression profiling of tumors from mice treated with MBS8 monotherapy show induction of the innate immune response at early time points with presence of high number of neutrophils and dendritic cells. An adaptive immune response is seen at later time points dominated by T-cells, which all-together mediate the anti-tumor effect of the MBS8 treatment.

### Example 22: Acute Effects on the Tumor Microenvironment Following MBS8 Treatment

The acute effects of intravenous injection of 200 nmol MBS8 was evaluated on tumors by multicolor flow cytometry. Mice bearing CT26 tumors were treated with MBS8 and tumors evaluated by flow cytometry 1, 3 and 6 hours after injection. Erythrocytes were determined based on size and Ter-119 expression and excluded from further analyses. All further analyses were determined based on lack of Ter-119 expression and based on size and being stained as alive. CD8+ T cells were defined as CD45+CD3+CD8+. Erythrocytes comprised the majority of cells in the tumors 6 hours after injection of MBS8 (FIG. 17A). Additionally, the tumor microenvironment is characterized by a decrease in immune cells (CD45⁺) and a remarkable and surprising drop in cancer cells 6 hours after injection of MBS8 (FIG. 17B, graph 1 and 2). Although immune cells were decreased in the tumor, there was a strong increase in neutrophils 6 h after treatment (FIG. 17C, graph 1), whereas T-cells, NKT and NK cells did not significantly change (FIG. 17C, graph 2-5). However, when immune activation was monitored on these cell subsets based on CD69-expression, T-cells, NKT and NK cells showed a significant activation 3 h after treatment (FIG. 17D, graph 1-5).

### Conclusion

MBS8 injected intravenously induce enrichment of neutrophils and erythrocytes in the tumors 6 h after treatment and is accompanied by decrease in viable tumor cells and immune cells, whereas significant activation is seen for immune cells; CD8 T-cells, NKT-cells and NK cells.

### Example 23: Effects on the Tumor Microenvironment and Spleens Following TLR7 Micelles

The effects of intravenous injection of MBS6 on the tumor microenvironment was evaluated by multicolor flow cytometry. Mice bearing CT26 tumors were treated on day 15 after cancer cell inoculation with 200 nmol MBS6 every fourth day and tumors and spleens analyzed two days after the second injection and 6 days after the first injection (day 21). Viable cells were identified based on cell size and lack of staining by a viability dye. All further analyses were based on viable cells. Neutrophils were identified as CD45⁺ CD11b⁺ Ly6g⁺. Tumor associated macrophages (TAMs) were identified as CD45⁺ Ly6g⁻ CD11c⁺ CD11b⁺ CD64^{high}. Patrolling monocytes (PMos) were identified as CD45⁺ Ly6g⁻ CD11b⁺ CD11c⁻ Ly6c⁻ CD64+. monocytic myeloid-derived suppressor cells (Mo-MDSCs) were identified as CD45⁺ Ly6g⁻ CD11b⁺ CD11c⁻ Ly6c^{high}. Classical DC (cDC)1s were identified as CD45⁺ Ly6g⁻ CD11c⁺ CD64^{low} CD11b^{low} XCR1⁺. cDC2s were identified as CD45⁺ Ly6g⁻ CD11c⁺ CD64^{low} XCR1⁻ CD11b^{high}. Plasmacytoid DCs (pDCs) were identified as CD45⁺ Ly6g⁻ CD11c⁺ CD64^{low} XCR1⁻ CD11b⁻ Ly6c^{high} Siglec-H⁺. CD8⁺ T cells were identified as CD45⁺ side scatter^{Ow} CD3⁺ CD4⁻ CD8⁺. Cells per 100 mg were calculated based on population of interest, tumor weight and total cells. Tumor weights were lower compared to untreated tumors (FIG. 18A, p=0.056). Additionally, the cellular viability in tumors were significantly reduced after treatment with MBS6, which was barely reflected in spleens (FIG. 18B, graph 1 and 2). Of the viable cells, MBS6 induced a strong increase in immune cells (CD45⁺) (FIG. 18C). This is in contrast to what was seen in FIG. 17C, but can be explained by the fact that this time point was 6 days after first treatment. Neutrophils were highly enriched within the tumor microenvironment as seen already after 6 h in FIG. 17 (relative to the depletion of other cell types) following MBS6 treatment which was not reflected in spleens (FIG. 18D, graph 1 and 2). All other investigated immune populations and cancer cells were strongly decreased in the tumor microenvironment (FIG. 18E, graph 1-8).

### Conclusion

Intravenous injection of MBS6 strongly decreases the cellular viability within the tumor microenvironment while not affecting the viability in spleens. Additionally, MBS6 induced a strong increase in neutrophils in the tumor microenvironment which was not observed in spleens.

### Example 24: Optimal Dosing Schedule for MBS8 and Doxil

Dosing schedule when using combination treatments with MBS8 and chemotherapy is critical in order to achieve the optimal tumor control. We wanted to test if Doxil treatment was improved in combination with MBS8, and in that case how MBS8 should be dosed to achieve the optimal response (FIG. 19). Doxil treatment alone showed a tumor growth delay but no mice in complete remission, and with a tumor growth inhibition (TGI), compared to Vehicle treated mice at 80% (FIG. 19). When MBS8 was administered starting two days after last Doxil treatment (MBS8 postdose), there was a significant tumor growth inhibition of 90% vs vehicle treated mice, and a significant anti-tumor activity compared to Doxil alone (p=0.0007). However, when MBS8 was dosed initially at the same day as Doxil, (MBS8 pre+postdose), all mice were in complete remission with a TGI of 98%, and significantly better antitumor activity than for Doxil alone or when MBS8 was only dosed post-Doxil treatment.

### Conclusion

These data show that MBS8 immunotherapy is significantly more efficient when a pre-dose is applied the same day as chemotherapy, and then with a follow up treatment after chemotherapy is terminated, compared to the setting where immunotherapy is initiated prior to chemotherapy.

### Example 25: Micelles Show No Sign of Accelerated Blood Clearance (ABC)

### In Mice:

Mean (+/−SD) plasma concentration-time curves of MBS8 in female and male rats on Day 1 (at first dose) and Day 15 (one week after last dose of two doses 1 week apart) after intravenous infusion administration over a period of 1 hour of 3 mg/kg/day MBS8 (1V270) is shown in FIG. 20 at a linear scale (A) and semi-logarithmic scale (B). This example demonstrates indifferent kinetics of MBS8 administered after 1 day and 15 days (third dose), respectively. This observation supports that the micelles of the present invention do not trigger accelerated blood clearance (ABC).

### In Cynomolgus Monkeys:

Overlay of individual plasma concentration-time curves of 1V270 in 1 female and 1 male cynomolgus monkey is shown in FIG. 21 on Day 1 (A, first dose) and Day 15 (B, third dose after one weekly dose for two doses) after intravenous infusion administration over a period of 1 hour of 0.3, 1 and 3 mg/kg/day MBS8 (1V270) at a linear scale. This example demonstrates indifferent kinetics of MBS8 administered after 1 day and third dose after 15 days, respectively. This observation supports that the micelles of the present invention do not trigger accelerated blood clearance (ABC).

### Conclusion

In both mice and cynomolgus monkeys, the micelles of the present disclosure did not trigger accelerated blood clearance (ABC) when infused at Day 1 (first dose) and Day 15 (third dose). This supports the significantly improved therapeutic utility of micelles at least in comparison with liposomes known to trigger an ABC event.

1. A nanoparticle composition comprising:
a toll-like receptor 7 (TLR7) agonist of formula (I), formula (II), formula (III), formula (IV), or Telratolimod; 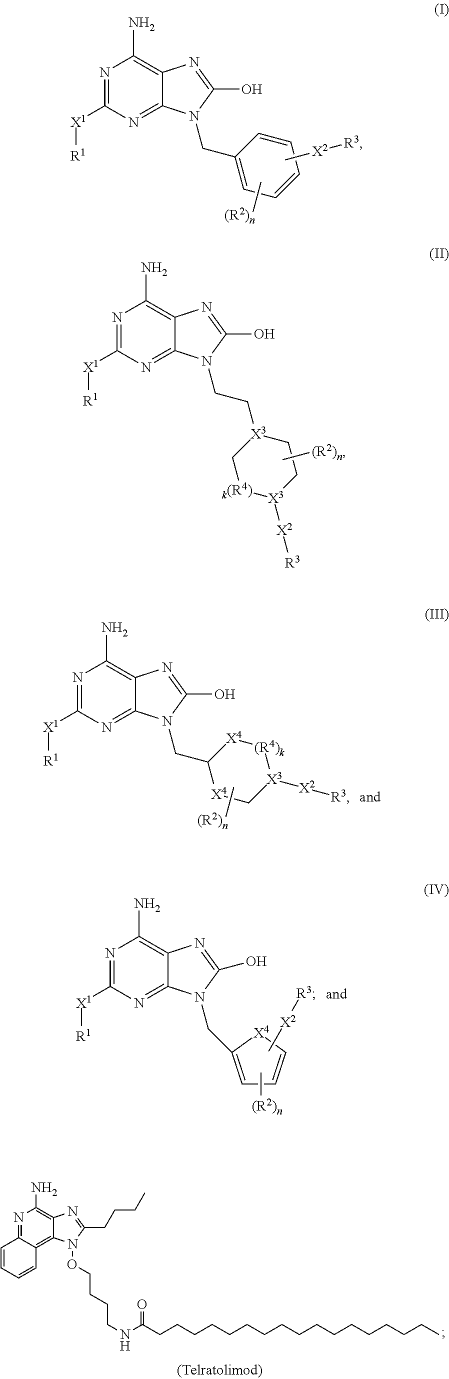
wherein X¹ is —O—, —S—, or —NR^{C};
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R¹ is hydrogen, C₁₋₁₀alkyl, or substituted C₁₋₁₀alkyl; or R^{C} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxy C₁₋₆alkylene, amino, cyano, halo, or aryl;
n is 0, 1, 2, 3 or 4;
X² is a bond or a linking group; and
R³ is a lipid;
X³ is —N— or —CH—;
R⁴ is —CH₂— or —CH(R²)—; and
k is 0 or 1;
X⁴ is —O—, —S—, —NH—, —N(R^{d})—, —CH₂—, or —CH(R²)—;
each R^{d} is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)O(C₁-C₆)alkyl (alkoxycarbonyl), substituted —C(O)O(C₁-C₆)alkyl, —C(O)NR^{a}R^{b} (carbamoyl);
or a tautomer thereof;
or a pharmaceutically acceptable salt or solvate thereof, and
wherein the ring system of formula (II) is a piperidine ring with one heteroatom being an N atom and with the N-atom of the piperidine ring adjacent to X², and
wherein the purine group in any of Formula (I), (II), (III), or (IV) is subject to tautomeric rearrangements;
and an amphiphilic micelle-forming agent; and
wherein the molar ratio between the amphiphilic micelle-forming agent and the TLR7 agonist is from 50:50 to 99.5:0.5.

2. The nanoparticle composition according to claim 1, wherein the diameter of the micelle is between 5 nm and 25 nm, such as between 6 nm and 24 nm, such as between 7 nm and 23 nm, such as between 8 nm and 22 nm, such as between 9 nm and 21 nm, such as between 10 nm and 20 nm, such as between 11 nm and 19 nm, such as between 12 nm and 18 nm, such as between 13 nm and 17 nm, such as between 14 nm and 16 nm, such as 15 nm.

3. The nanoparticle composition according to any one of the preceding claims, wherein the TLR7 agonist is of formula (I): 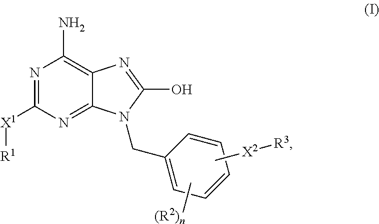
wherein X¹ is —O—, —S—, or —NR^{C};
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R^{C} is hydrogen, C₁₋₁₀alkyl, or substituted C₁₋₁₀alkyl; or R^{C} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxy C₁₋₆alkylene, amino, cyano, halo, or aryl;
n is 0, 1, or 2;
X² is a bond or a linking group; and
R³ is a lipid;
or a tautomer thereof;
or a pharmaceutically acceptable salt or solvate thereof, and
wherein the purine group is subject to tautomeric rearrangements.

4. The nanoparticle composition according to any one of the preceding claims, wherein the TLR7 agonist is of formula (I): 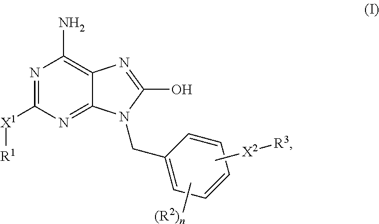
wherein X¹ is —O—, —S—, or —NR^{C};
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R^{C} is hydrogen, C₁₋₁₀alkyl, or substituted C₁₋₁₀alkyl; or R^{C} and R¹ taken together with the nitrogen to which they are attached form a heterocyclic ring or a substituted heterocyclic ring;
R² is absent;
n is 0;
X² is a bond or a linking group; and
R³ is a lipid;
or a tautomer thereof;
or a pharmaceutically acceptable salt or solvate thereof, and
wherein the purine group is subject to tautomeric rearrangements.

5. The nanoparticle composition according to any one of the preceding claims, wherein X² is selected from the group consisting of: a bond, —O—, —C(O)-(carbonyl), (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₅-C₁₀)aryl, —C(O)OH (carboxyl), —C(O)O(C₁-C₅)alkyl (alkoxycarbonyl), substituted —C(O)O(C₁-C₆)alkyl, —NR^{a}R^{b}, —C(O)NR^{a}R^{b} (carbamoyl); and
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
and wherein
X¹ is —O—, —S—, or —NR^{C};
R¹ is hydrogen, (C₁-C₆)alkyl, or substituted (C₁-C₆)alkyl;
R^{C} is hydrogen, C₁₋₆alkyl, or substituted C₁₋₆alkyl; or R^{C} and R¹ taken together with the nitrogen to which they are attached form a heterocyclic ring or a substituted heterocyclic ring.

6. The nanoparticle composition according to any one of the preceding claims, wherein R³ is a lipid selected from the group consisting of: a phospholipid comprising one or two carboxylic esters; a gonane, such as cholesterol; a saccharolipid; and a glyceride.

7. The nanoparticle composition according to any one of the preceding claims, wherein R³ is a phospholipid comprising one or two carboxylic esters.

8. The nanoparticle composition according to any one of the preceding claims, wherein the TLR7 agonist is selected from the group consisting of: 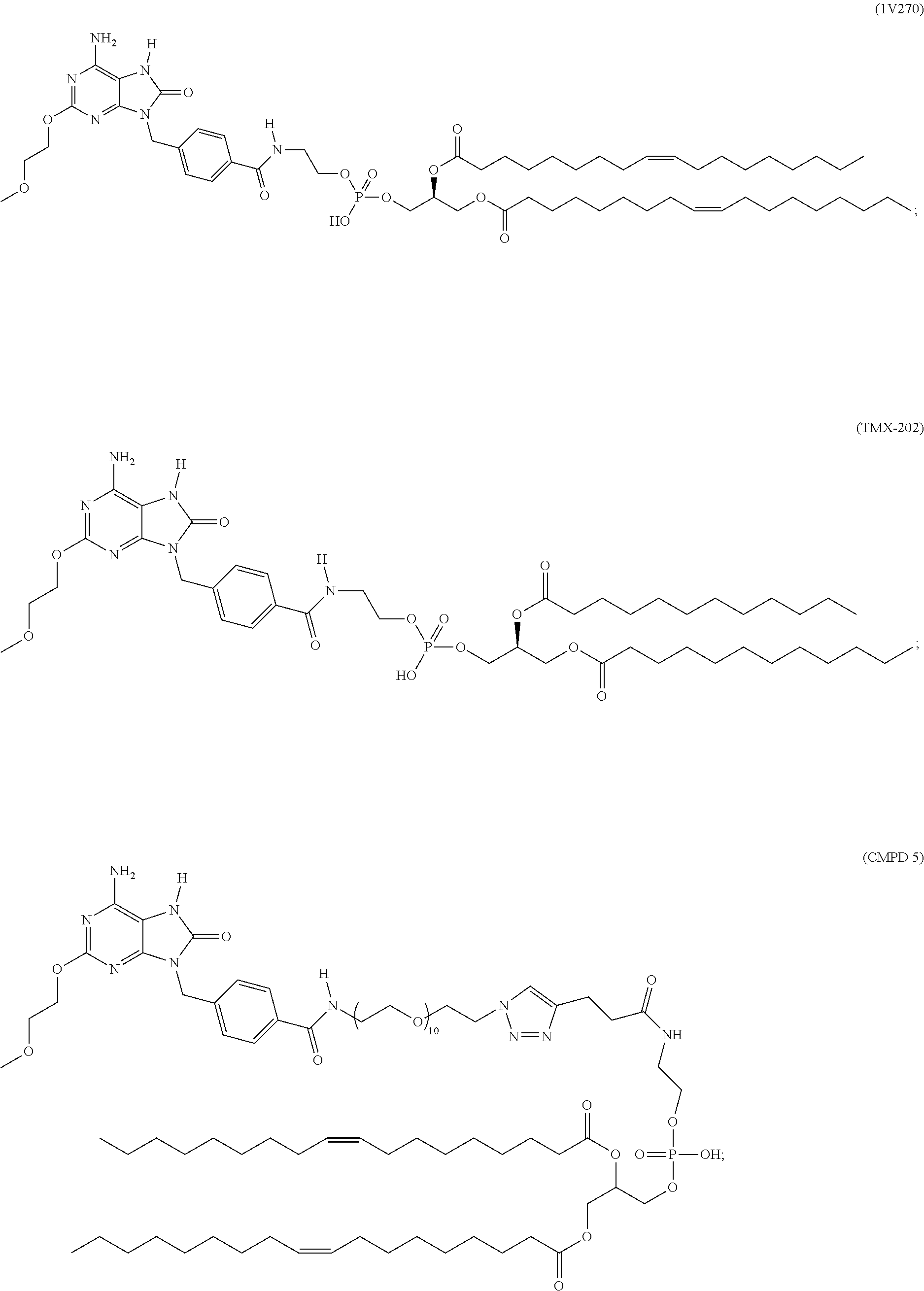 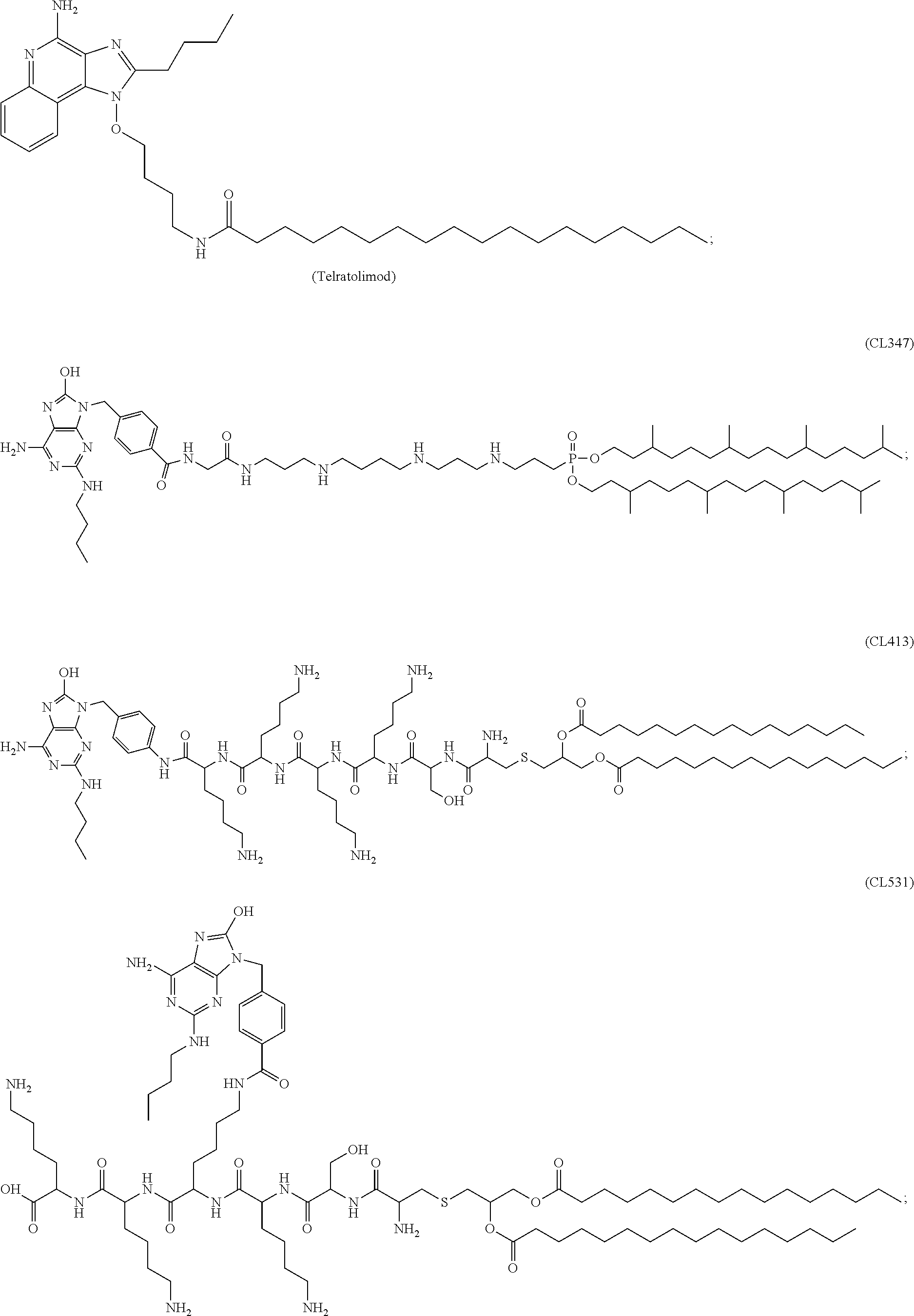
a tautomer thereof, and a pharmaceutically acceptable salt thereof.

9. The nanoparticle composition according to any one of the preceding claims, wherein the amphiphilic micelle-forming agent is selected from the group consisting of: a poloxamer, a poloxamine, a PEG-polyester, a PEG-polyanhydride, a PEG-poly-amino acid, a phospholipid, a polysorbate, and a polyoxyethylene alkyl ether.

10. The nanoparticle composition according to any one of the preceding claims, wherein the PEG-polyester is selected from the group consisting of: a PEG-poly(lactic acid) (PEG-PLA), a PEG-poly(lactic-co-glycolic acid) (PLGA), and a PEG-poly(ε-caprolactone) (PCL).

11. The nanoparticle composition according to any one of the preceding claims, wherein the PEG-polyanhydride is a PEG-polysebacic anhydride (PSA).

12. The nanoparticle composition according to any one of the preceding claims, wherein the PEG-poly-amino acid is selected from the group consisting of: a PEG-poly(L-histidine), a PEG-poly(L-aspartic acid), a PEG-poly(L-asparagine), a PEG-poly(L-glutamic acid), a PEG-poly(L-glutamine), and a PEG-poly(L-lysine).

13. The nanoparticle composition according to any one of the preceding claims, wherein the amphiphilic micelle-forming agent is a phospholipid conjugated to polyethylene glycol (PEG).

14. The nanoparticle composition according to any one of the preceding claims, wherein the phospholipid conjugated to PEG is conjugated via a carbonyl group.

15. The nanoparticle composition according to any one of the preceding claims, wherein the size of PEG is between PEG350 and PEG5000, for example between PEG550 and PEG4000, for example between PEG750 and PEG3000, such as between PEG1000 and PEG3000, preferably the size of the PEG is PEG2000.

16. The nanoparticle composition according to any one of the preceding claims, wherein the phospholipid comprises one or more alkyl chains that are C8-C24 alkyl(s), such as C10-C22, such as C12-C20, preferably C14-C18, most preferred C16-C18 saturated alkyl chains or unsaturated alkyl chains.

17. The nanoparticle composition according to any one of the preceding claims, wherein the phospholipid comprises a phosphatidylethanolamine (PE), a phosphatidylcholine (PC), a phosphatidylserine (PS), a phosphatidylglycerol (PG), a phosphatidylinositol (PI), a phosphatidic acid (PA), a bisphosphatidyl glycerol (DPG), or a phosphatidyl alcohol.

18. The nanoparticle composition according to any one of the preceding claims, wherein the phosphatidylethanolamine is selected from the group consisting of 1,2-dioleoyl-phosphatidylethanolamine, 1,2-dipalmitoyl-phosphatidylethanolamine, 1,2-dimyristoyl-phosphatidylethanolamine, 1,2-distearoyl-phosphatidylethanolamine, 1-oleoyl-2-palmitoyl-phosphatidylethanolamine, 1-oleoyl-2-stearoyl-phosphatidylethanolamine, 1-palmitoyl-2-oleoyl-phosphatidylethanolamine, and 1-stearoyl-2-oleoyl-phosphatidylethanolamine.

19. The nanoparticle composition according to any one of the preceding claims, wherein the phospholipid conjugated to PEG is selected from the group consisting of: 1,2-dioleoyl-sn-glycero-3-phosphoethanolamine (DOPE)-PEG, 1,2-distearoyl-sn-glycero-3-phosphoethanolamine (DSPE)-PEG, 1,2-dimyristoyl-sn-glycero-3-phosphoethanolamine (DMPE)-PEG, and 1,2-dipalmitoyl-sn-glycero-3-phosphoethanolamine (DPPE)-PEG.

20. The nanoparticle composition according to any one of the preceding claims, wherein the phospholipid conjugated to PEG is: 1,2-distearoyl-sn-glycero-3-phosphoethanolamine (DSPE)-PEG.

21. The nanoparticle composition according to any one of the preceding claims, wherein the amphiphilic micelle-forming agent is DSPE-PEG2000.

22. The nanoparticle composition according to any one of the preceding claims, wherein the molar ratio between the amphiphilic micelle-forming agent and the TLR7 agonist is from 60:40 to 99:1.

23. The nanoparticle composition according to any one of the preceding claims, wherein the molar ratio between the amphiphilic micelle-forming agent and the TLR7 agonist is from 70:30 to 98:2.

24. The nanoparticle composition according to any one of the preceding claims, wherein the molar ratio between the amphiphilic micelle-forming agent and the TLR7 agonist is from 80:20 to 95:5, for example 95:5, 90:10, or 80:20.

25. The nanoparticle composition according to any one of the preceding claims, wherein the molar ratio between the amphiphilic micelle-forming agent and the TLR7 agonist is 90:10.

26. The nanoparticle composition according to any one of the preceding claims, wherein the composition comprises between 1% and 25% molar concentration of TLR7 agonist, such as 1%, such as 2%, such as 3%, such as 4%, such as 5%, such as 6%, such as 7%, such as 8%, such as 9%, such as 10%, such as 11%, such as 12%, such as 13%, such as 14%, such as 15%, such as 16%, such as 17%, such as 18%, such as 19%, such as 20%, such as 21%, such as 22%, such as 23%, such as 24%, such as 25%.

27. The nanoparticle composition according to any one of the preceding claims, wherein the TLR7 agonist has a structure according to formula (IA): 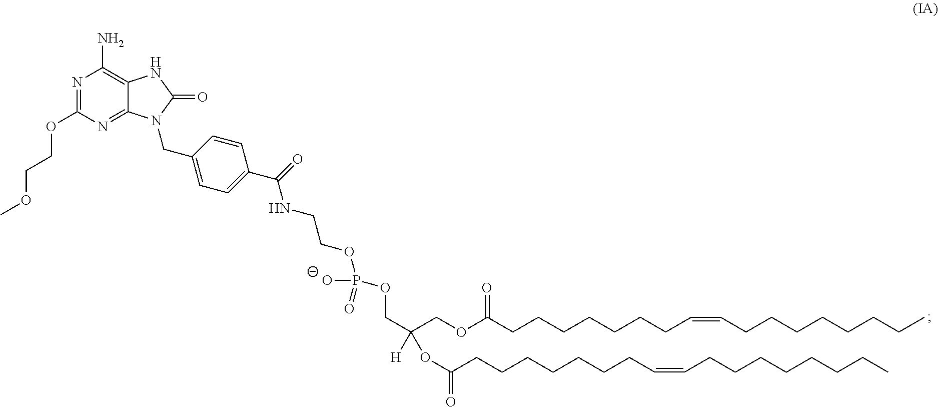 [SMILES: CCCCCCCC/C=C\CCCCCCCC(=O)OCC(COP(=O)([O-])OCCNC(=O)c1ccc(Cn2c(=O)[nH]c3c(N)nc(OCCOC)nc32)cc1)OC(=O)CCCCCCC/C=C\CCCCCCCC]
or a tautomer thereof;
or a pharmaceutically acceptable salt or solvate thereof.

28. A nanoparticle composition comprising:
a TLR7 agonist of formula (IA); 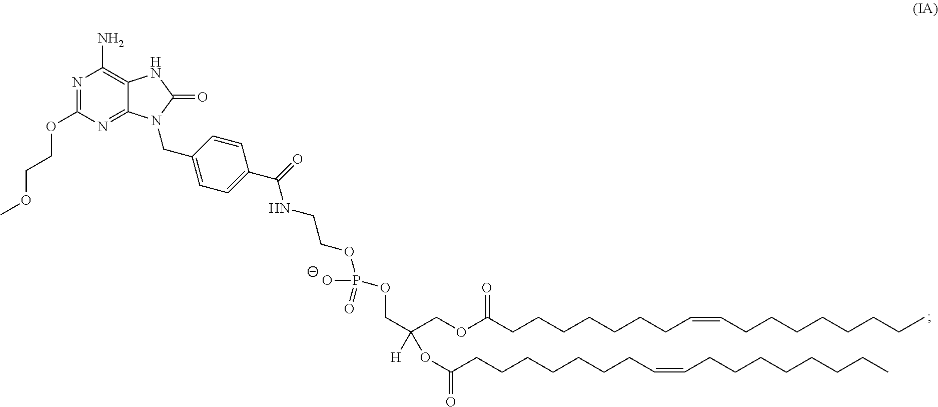 [SMILES: CCCCCCCC/C=C\CCCCCCCC(=O)OCC(COP(=O)([O-])OCCNC(=O)c1ccc(Cn2c(=O)[nH]c3c(N)nc(OCCOC)nc32)cc1)OC(=O)CCCCCCC/C=C\CCCCCCCC]
or a tautomer thereof;
or a pharmaceutically acceptable salt or solvate thereof; and
DSPE-PEG2000; and
the molar ratio between DSPE-PEG2000 and the TLR7 agonist is 95:5, 90:10, or 80:20.

29. The nanoparticle composition according to any one of the preceding claims, wherein the molar ratio between DSPE-PEG2000 and the TLR7 agonist of formula (IA) is 90:10 (MBS8).

30. The nanoparticle composition according to any one of the preceding claims, further comprising at least one further active ingredient.

31. The nanoparticle composition according to any one of the preceding claims, further comprising at least one antigen.

32. The nanoparticle composition according to any one of the preceding claims, wherein the nanoparticle composition is a micelle.

33. The nanoparticle composition according to any one of the preceding claims, wherein the nanoparticle composition is a liposome.

34. The nanoparticle composition according to any one of the preceding claims, wherein the nanoparticle composition is a nanodisc.

35. The nanoparticle composition according to any one of the preceding claims, wherein the nanoparticle composition is a lipoplex.

36. The nanoparticle composition according to any one of the preceding claims, wherein the nanoparticle composition is a lipidoid.

37. The nanoparticle composition according to any one of the preceding claims, wherein the nanoparticle composition is an emulsion.

38. A pharmaceutical composition comprising the nanoparticle composition according to any one of the preceding claims.

39. A nanoparticle composition as defined in any one of claims 1 to 37 or a pharmaceutical composition as defined in claim 38, for use in the prevention, treatment or amelioration of a disease or disorder.

40. The nanoparticle composition as defined in any one of claims 1 to 37 or pharmaceutical composition as defined in claim 38 for use according to any one of the preceding claims, wherein the disease or disorder is selected from the group consisting of: a cancer, an infectious disease, an inflammatory condition or disease, an autoimmune disease, and an allergy.

41. The nanoparticle composition as defined in any one of claims 1 to 37 or pharmaceutical composition as defined in claim 38 for use according to any one of the preceding claims, wherein the disease or disorder is cancer, such as colon cancer.

42. A method for in vivo activation of immune cells in a subject, comprising administering the nanoparticle composition as defined in any one of claims 1 to 37 or pharmaceutical composition as defined in claim 38 to said subject in an amount sufficient to activate said immune cells.

43. A method for treatment of cancer in a patient in need of treatment, comprising administering the nanoparticle composition as defined in any one of claims 1 to 37 to the patient.

44. The method according to any one of claims 42 to 43, further comprising administering a chemotherapeutic agent to the patient.

45. The method according to any one of claims 42 to 44, wherein the chemotherapeutic agent is selected from the group consisting of Doxorubicin, Doxil, Epirubicin, Cyclophosphamide, Bortezomib, and Oxaliplatin.

46. The method according to any one of claims 42 to 45, further comprising administering an immune check point inhibitor to the patient.

47. The method according to any one of claims 42 to 46, wherein the immune check point inhibitor is selected from the group consisting of: Atezolizumab, Avelumab, Durvalumab, Nivolumab, Tislelizumab, Pembrolizumab, and Ipilimumab.

48. The method according to any one of claims 42 to 47, wherein the nanoparticle composition is MBS8 and the immune check point inhibitor is selected from the group consisting of: Nivolumab and Pembrolizumab.

49. The method according to any one of claims 42 to 48, further comprising administering a monoclonal antibody targeting CD20 to the patient.

50. The method according to any one of claims 42 to 49, comprising administering MBS8 and a monoclonal antibody targeting CD20.

51. The method according to any one of claims 42 to 50, further comprising administering a monoclonal antibody targeting the epidermal growth factor receptor (EGFR) to the patient.

52. The method according to any one of claims 42 to 51, comprising administering MBS8 and a monoclonal antibody targeting the epidermal growth factor receptor (EGFR).

53. The method according to any one of claims 42 to 52, further comprising administering a monoclonal antibody targeting the Human Epidermal Growth Factor Receptor 2 (HER2) to the patient.

54. The method according to any one of claims 42 to 53, comprising administering MBS8 and a monoclonal antibody targeting the Human Epidermal Growth Factor Receptor 2 (HER2).

55. The method according to any one of claims 42 to 54, further comprising administering a monoclonal antibody targeting CD38, such as Daratumumab or Isatuximab, to the patient.

56. The method according to any one of claims 42 to 55, comprising administering MBS8 and a monoclonal antibody targeting CD38, such as Daratumumab or Isatuximab.

57. The method according to any one of claims 42 to 56, further comprising administering a monoclonal antibody targeting CD47, such as Magrolimab, to the patient.

58. The method according to any one of claims 42 to 57, comprising administering MBS8 and a monoclonal antibody targeting CD47, such as Magrolimab.

59. The method according to any one of claims 42 to 58, further comprising radiotherapy.
